(19) United States
(12) Patent Application Publication
(10) Pub. No.: US 2022/0041588 A1
(43) Pub. Date: Feb. 10, 2022

(54) SUBSTITUTED IMIDAZO[1,2-A]PYRIDINE AND [1,2,4]TRIAZOLO[1,5-A]PYRIDINE COMPOUNDS AS RET KINASE INHIBITORS

(71) Applicants: FOCHON PHARMACEUTICALS, LTD., Chongqing (CN); SHANGHAI FOCHON PHARMACEUTICAL CO., LTD., Shanghai (CN)

(72) Inventors: Chengxi HE, Chongqing (CN); Rui TAN, Chongqing (CN); Zuwen ZHOU, Chongqing (CN); Weipeng ZHANG, Chongqing (CN); Yunling WANG, Chongqing (CN); Qihong LIU, Chongqing (CN); Xingdong ZHAO, Chongqing (CN); Yanxin LIU, Chongqing (CN); Yuwei GAO, Chongqing (CN); Shu LIN, San Leandro, CA (US); Weibo WANG, Moraga, CA (US)

(21) Appl. No.: 17/280,267

(22) PCT Filed: Sep. 26, 2019
(86) PCT No.: PCT/CN2019/108164

§ 371 Date: Mar. 26, 2021

Related U.S. Application Data
(60) Provisional application No. 62/737,535, filed on Sep. 27, 2018.
(60) Provisional application No. 62/824,443, filed on Mar. 27, 2019.

(51) Int. Cl. C07D 471/04; A61K 45/06; A61K 31/496; A61K 31/437; A61K 31/5377; A61K 31/501

(52) U.S. Cl. CPC C07D 471/04; A61K 45/06; A61K 31/501; A61K 31/437; A61K 31/5377; A61K 31/496

(57) ABSTRACT

Provided are certain RET inhibitors, pharmaceutical compositions thereof, and methods of use thereof.

This application claims the priority to the U.S. provisional application Nos. 62/737,535, and 62/824,443, each of which is incorporated herein by reference in its entirety.

### FIELD OF THE INVENTION

Provided are certain compounds or pharmaceutically acceptable salts thereof which can inhibit RET tyrosine kinases and may be useful for the treatment of hyper-proliferative diseases like cancer and inflammation, or immune and autoimmune diseases.

### BACKGROUND OF THE INVENTION

Hyper-proliferative diseases like cancer and inflammation are attracting the scientific community to provide therapeutic benefits. In this regard efforts have been made to identify and target specific mechanisms which play a role in proliferating the diseases.

The rearranged during transfection (RET) kinase is a single-pass transmembrane receptor tyrosine kinase. RET plays important role for normal development, maturation and maintenance of a variety of tissues and cell types. RET has the classical structure of a receptor tyrosine kinase: a cysteine-rich cadherin-like extracellular domain, a transmembrane region and an intracellular region that catalyzes tyrosine kinase. RET signaling is activated by binding of a group of soluble proteins of the glial cell line-derived neurotrophic factor (GDNF) family ligands (GFLs), which also includes neurturin (NRTN), artemin (ARTN) and persephin (PSPN). GFLs first bind to an additional co-receptor of RET, which is one of four GDNF family receptor-α (GFRu) family members. Subsequently, the ligand-co-receptor complex binds to the extracellular domain of RET to induce RET dimerization, phosphorylation, and activation of the downstream signal transduction pathways via PI3K/Akt/mTOR, RAS/MAPK/ERK, or to recruitment of the CBL family of ubiquitin ligases.

Alterations in RET gene, including gene fusions and single nucleotide alterations, enhance the function of RET signaling in a number of ways, further promoting the activation of kinases and the transformation of proto-oncogenes. Therefore, RET gene alteration potentiates many aberrant physiological processes that negatively impact human health. Aberrant RET expression and/or activation is closely associated with the occurrence of various diseases, including medullary thyroid cancer, papillary thyroid cancer, multiple endocrine neoplasia type 2, non-small cell lung cancer, gastrointestinal disorders such as irritable bowel syndrome and etc. RET gene alterations can serve as predictive biomarker for targeted therapy. It has been shown that the inhibitors of RET signaling pathway serve as effective treatment for multiple pre-clinical animal model of cancer. In addition, the on-going clinical development of selective RET inhibitors have been demonstrated to be beneficial among patients whose tumors harbor RET gene alterations.

Although RET inhibitors were disclosed in the arts, e.g. WO2009099801 and WO2009003136, many suffer from short half-life or toxicity. Therefore, there is a need for new RET inhibitors that have at least one advantageous property selected from potency, stability, selectivity, toxicity and pharmacodynamics properties as an alternative for the treatment of hyper-proliferative diseases. In this regard, a novel class of RET inhibitors is provided herein.

### DISCLOSURE OF THE INVENTION

Disclosed herein are certain novel compounds, pharmaceutically acceptable salts thereof, and pharmaceutical compositions thereof, and their use as pharmaceuticals.

In one aspect, disclosed herein is a compound of formula (I):

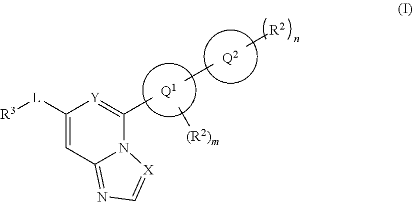

or a pharmaceutically acceptable salt thereof, wherein:

Q¹ is selected from aryl and heteroaryl;

Q² is heterocyclyl;

X is selected from CR⁴ and N;

Y is selected from CR⁵ and N;

L is selected from a bond, —(CR^{C0}R^{D0})ᵤ—, —(CR^{C0}R^{D0})ᵤO(CR^{C0}R^{D0})ₜ—, —(CR^{C0}R^{D0})ᵤNR^{A0}(CR^{C0}R^{D0})ₜ—, —(CR^{C0}R^{D0})ᵤS(CR^{C0}R^{D0})ₜ—, —(CR^{C0}R^{D0})ᵤC(O)NR^{A0}(CR^{C0}R^{D0})ₜ—, —(CR^{C0}R^{D0})ᵤNR^{A0}C(O)(CR^{C0}R^{D0})ₜ—, —(CR^{C0}R^{D0})ᵤNR^{A0}C(O)NR^{B0}(CR^{C0}R^{D0})ₜ—, —(CR^{C0}R^{D0})ᵤS(O)ᵣ(CR^{C0}R^{D0})ₜ—, —(CR^{C0}R^{D0})ᵤS(O)ᵣNR^{A0}(CR^{C0}R^{D0})ₜ—, —(CR^{C0}R^{D0})ᵤNR^{A0}S(O)ᵣ(CR^{C0}R^{D0})ₜ—, and —(CR^{C0}R^{D0})ᵤNR^{A0}S(O)ᵣNR^{B0}(CR^{C0}R^{D0})ₜ—;

each R¹ is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═NR^{E1})R^{A1}, —C(═N—OR^{B1})R^{A1}, —C(O)OR^{A1}, —OC(O)R^{A1}, —C(O)NR^{A1}R^{B1}, —NR^{A1}C(O)R^{B1}, —C(═NR^{E1})NR^{A1}R^{B1}, —NR^{A1}C(═NR^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1})NR^{A1}R^{B1}, —S(O)ᵣR^{A1}, —S(O)(═NR^{E1})R^{B1}, —N═S(O)R^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}, —S(O)ᵣNR^{A1}R^{B1}, —S(O)(═NR^{E1})NR^{A1}R^{B1}, —NR^{A1}S(O)₂NR^{A1}R^{B1}, —NR^{A1}S(O)(═NR^{E1})NR^{A1}R^{B1}, —P(O)R^{A1}R^{B1} and —P(O)(OR^{A1})(OR^{B1}), wherein alkyl, alkenyl, alkynyl, cycloalkyl, heterocyclyl, aryl and heteroaryl are each unsubstituted or substituted with at least one substituent, independently selected from R^{X};

each R² is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —NR^{A2}C(O)R^{B2}, —C(═NR^{E2})NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2})NR^{A2}R^{B2}, —S(O)ᵣR^{A2}, —S(O)(═NR^{E2})R^{B2}, —N═S(O)R^{A2}R^{B2}, —S(O)₂OR^{A2}, —OS(O)₂R^{A2}, —NR^{A2}S(O)ᵣR^{B2}, —NR^{A2}S(O)(═NR^{E2})R^{B2}, —S(O)ᵣNR^{A2}R^{B2}, —S(O)(═NR^{E2})NR^{A2}R^{B2}, —NR^{A2}S(O)₂NR^{A2}R^{B2}, —NR^{A2}S(O)(═NR^{E2})NR^{A2}R^{B2}, —P(O)R^{A2}R^{B2} and —P(O)(OR^{A2})(OR^{B2}), wherein alkyl, alkenyl, alkynyl, cycloalkyl, heterocyclyl, aryl and heteroaryl are each unsubstituted or substituted with at least one substituent, independently selected from R^{X};

R³ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —NR^{A3}C(O)R^{B3}, —C(═NR^{E3})NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3})NR^{A3}R^{B3}, —S(O)ᵣR^{A3}, —S(O)(═NR^{E3})R^{B3}, —N═S(O)R^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3})NR^{A3}R^{B3}, —NR^{A3}S(O)₂NR^{A3}R^{B3}, —NR^{A3}S(O)(═NR^{E3})NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R⁶;

R⁴ and R⁵ are independently selected from hydrogen, halogen, CN, C₁₋₁₀ alkyl and C₃₋₁₀ cycloalkyl, wherein alkyl and cycloalkyl are unsubstituted or substituted with at least one substituent, independently selected from R^{X};

R⁶ is selected from hydrogen, halogen, OH, C₁₋₁₀ alkyl, C₃₋₁₀ cycloalkyl, C₃₋₁₀ cycloalkyl-C₁₋₄ alkyl, heterocyclyl and heterocyclyl-C₁₋₄ alkyl, wherein alkyl, cycloalkyl and heterocyclyl are unsubstituted or substituted with at least one substituent, independently selected from R^{X};

each R^{A0}, R^{A1}, R^{A2}, R^{A3}, R^{B0}, R^{B1}, R^{B2} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X};

or each “R^{A0} and R^{B0}”, “R^{A1} and R^{B1}”, “R^{A2} and R^{B2}” or “A³ and R^{B3}” together with the atom(s) to which they are attached form a heterocyclic ring of 4 to 12 members containing 0, 1 or 2 additional heteroatoms independently selected from oxygen, sulfur, nitrogen and phosphorus and optionally substituted with 1, 2 or 3 R^{X} groups;

each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X};

or R^{C0} and R^{D0} together with the carbon atom(s) to which they are attached form a ring of 3 to 12 members containing 0, 1 or 2 heteroatoms independently selected from oxygen, sulfur and nitrogen and optionally substituted with 1 2 or 3 R^{X} groups;

each R^{E1}, R^{E2} and R^{E3} are independently selected from hydrogen, C₁₋₁₀ alkyl, CN, NO₂, —OR^{a1}, —SR^{a1}, —S(O)ᵣR^{a1}, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from R^{X};

each R^{X} is independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1}, —(CR^{c1}R^{d1})ₜC(═N—OR^{b1})R^{a1}, —(CR^{c1}R^{d1})ₜC(O)OR^{b1}, —(CR^{c1}R^{d1})ₜOC(O)R^{b1}, —(CR^{c1}R^{d1})ₜC(O)NR^{a1}R^{b1}, —(CR^{c1}R^{d1})ₜNR^{a1}C(O)R^{b1}, —(CR^{c1}R^{d1})ₜC(═NR^{e1})NR^{a1}R^{b1}, —(CR^{c1}R^{d1})ₜNR^{a1}C(═NR^{e1})R^{b1}, —(CR^{c1}R^{d1})ₜOC(O)NR^{a1}R^{b1}, —(CR^{c1}R^{d1})ₜNR^{a1}C(O)OR^{b1}, —(CR^{c1}R^{d1})ₜNR^{a1}C(O)NR^{a1}R^{b1}, —(CR^{c1}R^{d1})ₜNR^{a1}C(S)NR^{a1}R^{b1}, —(CR^{c1}R^{d1})ₜNR^{a1}C(═NR^{e1})NR^{a1}R^{b1}, —(CR^{c1}R^{d1})ₜS(O)ᵣR^{b1}, —(CR^{c1}R^{d1})ₜS(O)(═NR^{e1})R^{b1}, —(CR^{c1}R^{d1})ₜN═S(O)R^{a1}R^{b1}, —(CR^{c1}R^{d1})ₜS(O)₂OR^{b1}, —(CR^{c1}R^{d1})ₜOS(O)₂R^{b1}, —(CR^{c1}R^{d1})ₜNR^{a1}S(O)ᵣR^{b1}, —(CR^{c1}R^{d1})ₜNR^{a1}S(O)(═NRe)R^{b1}, —(CR^{c1}R^{d1})ₜS(O)ᵣNR^{a1}R^{b1}, —(CR^{c1}R^{d1})ₜS(O)(═NR^{e1})NR^{a1}R^{b1}, —(CR^{c1}R^{d1})ₜNR^{a1}S(O)₂NR^{a1}R^{b1}, —(CR^{c1}R^{d1})ₜNR^{a1}S(O)(═NR^{e1})NR^{a1}R^{b1}, —(CR^{c1}R^{d1})ₜP(O)R^{a1}R^{b1} and —(CR^{c1}R^{d1})ₜP(O)(OR^{a1})(OR^{b1}), wherein alkyl, alkenyl, alkynyl, cycloalkyl, heterocyclyl, aryl and heteroaryl are each unsubstituted or substituted with at least one substituent, independently selected from R^{Y};

each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};

or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{Y} groups;

each R^{c1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};

or R^{c1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 R^{Y} groups;

each R^{e1} is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR^{a2}R^{b2} and —C(O)NR^{a2}R^{b2};
each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})ₜNR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a2}, —(CR^{c2}R^{d2})ₜC(═N—OR^{b2})R^{a2}, —(CR^{c2}R^{d2})ₜC(O)OR^{b2}, —(CR^{c2}R^{d2})ₜOC(O)R^{b2}, —(CR^{c2}R^{d2})ₜC(O)NR^{a2}R^{b2}, —(CR^{c2}R^{d2})ₜNR^{a2}C(O)R^{b2}, —(CR^{c2}R^{d2})ᵣC(═NR^{e2})NR^{a2}R^{b2}, —(CR^{c2}R^{d2})ᵣNR^{a2}C(═NR^{e2})R^{b2}, —(CR^{c2}R^{d2})ₜOC(O)NR^{a2}R^{b2}, —(CR^{c2}R^{d2})ᵣNR^{a2}C(O)OR^{b2}, —(CR^{c2}R^{d2})ᵣNR^{a2}C(O)NR^{a2}R^{b2}, —(CR^{c2}R^{d2})ᵣNR^{a2}C(S)NR^{a2}R^{b2}, —(CR^{c2}R^{d2})ᵣNR^{a2}C(═NR^{e2})NR^{a2}R^{b2}, —(CR^{c2}R^{d2})ₜS(O)ᵣR^{b2}, —(CR^{c2}R^{d2})ᵣS(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2})ₜOS(O)₂R^{b2}, —(CR^{c2}R^{d2})ₜNR^{a2}S(O)ᵣR^{b2}, —(CR^{c2}R^{d2})ₜNR^{a2}S(O)(═NR^{e2})R^{b2}, —(CR^{c2}R^{d2})ₜS(O)ᵣNR^{a2}R^{b2}, —(CR^{c2}R^{d2})ᵣS(O)(═NR^{e2})NR^{a2}R^{b2}, —(CR^{c2}R^{d2})ᵣNR^{a2}S(O)₂NR^{a2}R^{b2}, —(CR^{e2}R^{d2})ᵣNR^{a2}S(O)(═NR^{e2})NR^{a2}R^{b2}, —(CR^{e2}R^{d2})ᵣP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;

each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

or R^{a2} and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

each R^{c2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

or R^{c2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O)C₁₋₄ alkyl, —C(O)C₃₋₁₀ cycloalkyl, —C(O)OC₁_₄ alkyl, —C(O)OC₃₋₁₀ cycloalkyl, —C(O)N(C₁₋₄ alkyl)₂, —C(O)N(C₃₋₁₀ cycloalkyl)₂, —S(O)₂C₁₋₄ alkyl, —S(O)₂C₃₋₁₀ cycloalkyl, —S(O)₂N(C₁₋₄ alkyl)₂ and —S(O)₂N(C₃₋₁₀ cycloalkyl)₂;

m is selected from 1, 2 and 3;

n is selected from 1, 2 and 3;

each r is independently selected from 0, 1 and 2;

each t is independently selected from 0, 1, 2, 3 and 4;

each u is independently selected from 0, 1, 2, 3 and 4.

In one embodiment of formula (I), the invention provides a compound or a pharmaceutically acceptable salt thereof, wherein L is a bond, R³ is pyrazolyl, and the compound has the formula (II),

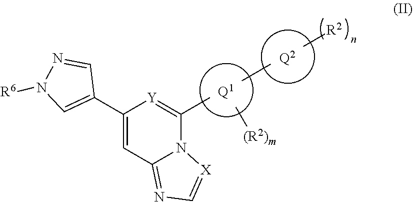

wherein Q¹, Q², X, Y, R¹, R², R⁶, n and m are as defined in formula (I).

In one embodiment of formula (I), the invention provides a compound or a pharmaceutically acceptable salt thereof, wherein L is O, and the compound has the formula (III),

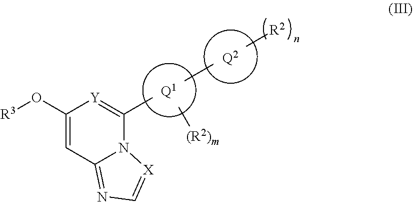

wherein Q¹, Q², X, Y, R¹, R², R³, n and m are as defined in formula (I).

In one embodiment of formula (II), the invention provides a compound or a pharmaceutically acceptable salt thereof, wherein:

when Y is CH, the compound has the formula (IV),

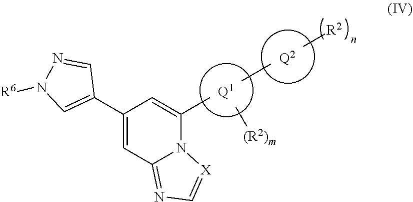

when Y is N, the compound has the formula (V),

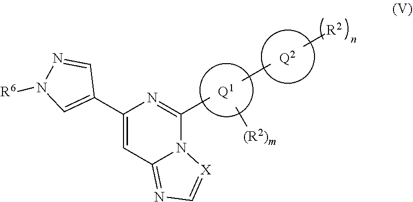

wherein Q¹, Q², X, R¹, R², R⁶, n and m are as defined in formula (I).

In yet another aspect, the present disclosure provides pharmaceutical compositions comprising a compound of formula (I) or at least one pharmaceutically acceptable salt thereof and a pharmaceutically acceptable excipient.

In yet another aspect, the disclosure provides methods for modulating RET kinase, comprising administering to a system or a subject in need thereof, a therapeutically effective amount of a compound of formula (I) or a pharmaceutically acceptable salt thereof or pharmaceutical compositions thereof, thereby modulating said RET kinase.

In yet another aspect, disclosed is a method to treat, ameliorate or prevent a condition which responds to inhibition of RET kinase comprising administering to a system or subject in need of such treatment an effective amount of a compound of formula (I) or a pharmaceutically acceptable salt thereof or pharmaceutical compositions thereof, and optionally in combination with a second therapeutic agent, thereby treating said condition.

Alternatively, the present disclosure provides the use of a compound of formula (I) or a pharmaceutically acceptable salt thereof in the manufacture of a medicament for treating a condition mediated by protein kinase. In particular embodiments, the compounds of the disclosure may be used alone or in combination with a second therapeutic agent to treat a condition mediated by RET kinase.

Alternatively, disclosed is a compound of formula (I) or a pharmaceutically acceptable salt thereof for treating a condition mediated by RET kinase.

Specifically, the condition herein includes but not limited to, an autoimmune disease, a transplantation disease, an infectious disease or a cell proliferative disorder.

Furthermore, the disclosure provides methods for treating a cell proliferative disorder, comprising administering to a system or subject in need of such treatment an effective amount of a compound of formula (I) or a pharmaceutically acceptable salt thereof or pharmaceutical compositions thereof, and optionally in combination with a second therapeutic agent, thereby treating said condition.

Alternatively, the present disclosure provides the use of a compound of formula (I) or a pharmaceutically acceptable salt thereof in the manufacture of a medicament for treating a cell-proliferative disorder. In particular examples, the compounds of the disclosure may be used alone or in combination with a chemotherapeutic agent to treat a cell proliferative disorder.

Specifically, the cell proliferative disorder disclosed herein includes but not limited to, lymphoma, osteosarcoma, melanoma, or a tumor of breast, renal, prostate, colorectal, thyroid, ovarian, pancreatic, neuronal, lung, uterine or gastrointestinal tumor.

In the above methods for using the compounds of the disclosure, a compound of formula (I) or a pharmaceutically acceptable salt thereof may be administered to a system comprising cells or tissues, or to a subject including a mammalian subject such as a human or animal subject.

### Certain Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail.

It is to be understood that the foregoing general description and the following detailed description are explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, CH₂O is equivalent to OCH₂.

The term “substituted” means that a hydrogen atom is replaced by a substituent. It is to be understood that substitution at a given atom is limited by valency.

The term “Cᵢ₋ⱼ” or “i-j membered” used herein means that the moiety has i-j carbon atoms or i-j atoms. For example, “C₁₋₆alkyl” means said alkyl has 1-6 carbon atoms. Likewise, C₃₋₁₀ cycloalkyl means said cycloalkyl has 3-10 carbon atoms.

When any variable (e.g. R) occurs at the structure of a compound over one time, it is defined independently at each case. Therefore, for example, if a group is substituted by 0-2 R, the group may be optionally substituted by at most two R and R has independent option at each case. Additionally, a combination of substituents and/or the variants thereof are allowed only if such a combination will result in a stable compound.

The expression “one or more” or “at least one” refers to one, two, three, four, five, six, seven, eight, nine or more.

Unless stated otherwise, the term “hetero” means heteroatom or heteroatom radical (i.e. a radical containing heteroatom), i.e. the atoms beyond carbon and hydrogen atoms or the radical containing such atoms. Preferably, the heteroatom(s) is independently selected from the group consisting of O, N, S, P and the like. In an embodiment wherein two or more heteroatoms are involved, the two or more heteroatoms may be the same, or part or all of the two or more heteroatoms may be different.

The term “alkyl”, employed alone or in combination with other terms, refers to branched or straight-chain saturated aliphatic hydrocarbon groups having the specified number of carbon atoms. Unless otherwise specified, “alkyl” refers to C₁₋₁₀ alkyl. For example, C₁₋₆, as in “C₁₋₆ alkyl” is defined to include groups having 1, 2, 3, 4, 5, or 6 carbons in a linear or branched arrangement. For example, “C₁₋₈ alkyl” includes but is not limited to methyl, ethyl, n-propyl, i-propyl, n-butyl, t-butyl, i-butyl, pentyl, hexyl, heptyl, and octyl.

The term “cycloalkyl”, employed alone or in combination with other terms, refers to a monocyclic or bridged saturated hydrocarbon ring system. The monocyclic cycloalkyl is a monocyclic hydrocarbon ring system containing 3-10 carbon atoms, zero heteroatoms and zero double bonds. Examples of monocyclic ring systems include but are not limited to cyclopropyl, cyclobutyl, cyclopentyl, cyclohexyl, cycloheptyl, and cyclooctyl. The bridged cycloalkyl is a polycyclic ring system containing 3-10 carbon atoms, which contains one or two alkylene bridges, each alkylene bridge consisting of one, two, or three carbon atoms, each linking two non-adjacent carbon atoms of the ring system. Cycloalkyl can be fused with aryl or heteroaryl group. In some embodiments, cycloalkyl is benzocondensed. Representative examples of such bridged cycloalkyl ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, bicyclo[4.2.1]nonane, tricyclo[3.3.1.0^{3,7}]nonane and tricyclo[3.3.1.1^{3,7}]decane (adamantane). The monocyclic or bridged cycloalkyl can be attached to the parent molecular moiety through any substitutable atom contained within the ring system.

The term “alkenyl”, employed alone or in combination with other terms, refers to a non-aromatic hydrocarbon radical, straight, branched or cyclic, containing 2-10 carbon atoms and at least one carbon to carbon double bond. In some embodiments, one carbon to carbon double bond is present, and up to four non-aromatic carbon-carbon double bonds may be present. Thus, “C₂_₆ alkenyl” means an alkenyl radical having 2-6 carbon atoms. Alkenyl groups include but are not limited to ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl”, employed alone or in combination with other terms, refers to a hydrocarbon radical, straight, branched or cyclic, containing 2-10 carbon atoms and at least one carbon to carbon triple bond. In some embodiments, up to three carbon-carbon triple bonds may be present. Thus, “C₂_₆ alkynyl” means an alkynyl radical having 2-6 carbon atoms. Alkynyl groups include but are not limited to ethynyl, propynyl, butynyl, and 3-methylbutynyl. The straight, branched or cyclic portion of the alkynyl group may contain triple bonds and may be substituted if a substituted alkynyl group is indicated.

The term “halogen” (or “halo”) refers to fluorine, chlorine, bromine and iodine.

The term “alkoxy”, employed alone or in combination with other terms, refers to an alkyl as defined above, which is single bonded to an oxygen atom. The attachment point of an alkoxy radical to a molecule is through the oxygen atom. An alkoxy radical may be depicted as —O-alkyl. The term “C₁₋₁₀ alkoxy” refers to an alkoxy radical containing 1-10 carbon atoms, having straight or branched moieties. Alkoxy group includes but is not limited to, methoxy, ethoxy, propoxy, isopropoxy, butoxy, pentyloxy, hexyloxy, and the like.

The term “cycloalkoxy”, employed alone or in combination with other terms, refers to cycloalkyl as defined above, which is single bonded to an oxygen atom. The attachment point of a cycloalkoxy radical to a molecule is through the oxygen atom. A cycloalkoxy radical may be depicted as —O-cycloalkyl. “C₃₋₁₀ cycloalkoxy” refers to a cycloalkoxy radical containing 3-10 carbon atoms. Cycloalkoxy can be fused with aryl or heteroaryl group. In some embodiments, cycloalkoxy is benzocondensed. Cycloalkoxy group includes but is not limited to, cyclopropoxy, cyclobutoxy, cyclopentyloxy, cyclohexyloxy, and the like.

The term “alkylthio”, employed alone or in combination with other terms, refers to an alkyl radical as defined above, which is single bonded to a sulfur atom. The attachment point of an alkylthio radical to a molecule is through the sulfur atom. An alkylthio radical may be depicted as —S-alkyl. The term “C₁₋₁₀ alkylthio” refers to an alkylthio radical containing 1-10 carbon atoms, having straight or branched moieties. Alkylthio group includes but is not limited to, methylthio, ethylthio, propylthio, isopropylthio, butylthio, hexylthio, and the like.

The term “cycloalkylthio”, employed alone or in combination with other terms, refers to cycloalkyl as defined above, which is single bonded to a sulfur atom. The attachment point of a cycloalkylthio radical to a molecule is through the sulfur atom. A cycloalkylthio radical may be depicted as —S-cycloalkyl. “C₃₋₁₀ cycloalkylthio” refers to a cycloalkylthio radical containing 3-10 carbon atoms. Cycloalkylthio can be fused with aryl or heteroaryl group. In some embodiments, cycloalkylthio is benzocondensed. Cycloalkylthio group includes but is not limited to, cyclopropylthio, cyclobutylthio, cyclohexylthio, and the like.

The term “alkylamino”, employed alone or in combination with other terms, refers to an alkyl as defined above, which is single bonded to a nitrogen atom. The attachment point of an alkylamino radical to a molecule is through the nitrogen atom. An alkylamino radical may be depicted as —NH(alkyl). The term “C₁₋₁₀ alkylamino” refers to an alkylamino radical containing 1-10 carbon atoms, having straight or branched moieties. Alkylamino group includes but is not limited to, methylamino, ethylamino, propylamino, isopropylamino, butylamino, hexylamoino, and the like.

The term “cycloalkylamino”, employed alone or in combination with other terms, refers to cycloalkyl as defined above, which is single bonded to a nitrogen atom. The attachment point of a cycloalkylamino radical to a molecule is through the nitrogen atom. A cycloalkylamino radical may be depicted as —NH(cycloalkyl). “C₃₋₁₀ cycloalkylamino” refers to a cycloalkylamino radical containing 3-10 carbon atoms. Cycloalkylamino can be fused with aryl or heteroaryl group. In some embodiments, cycloalkylamino is benzocondensed. Cycloalkylamino group includes but is not limited to, cyclopropylamino, cyclobutylamino, cyclohexylamino, and the like.

The term “di(alkyl)amino”, employed alone or in combination with other terms, refers to two alkyl as defined above, which are single bonded to a nitrogen atom. The attachment point of an di(alkyl)amino radical to a molecule is through the nitrogen atom. A di(alkyl)amino radical may be depicted as —N(alkyl)₂. The term “di(C₁₋₁₀ alkyl)amino” refers to a di(C₁₋₁₀ alkyl)amino radical wherein the alkyl radicals each independently contains 1-10 carbon atoms, having straight or branched moieties.

The term “aryl”, employed alone or in combination with other terms, refers to a monovalent, monocyclic-, bicyclic- or tricyclic aromatic hydrocarbon ring system having 6, 7, 8, 9, 10, 11, 12, 13 or 14 carbon atoms (a “C₆₋₁₄ aryl” group), particularly a ring having 6 carbon atoms (a “C₆ aryl” group), e.g. a phenyl group; or a ring having 10 carbon atoms (a “C₁₀ aryl” group), e.g. a naphthyl group; or a ring having 14 carbon atoms, (a “C₁₄ aryl” group), e.g. an anthranyl group. Aryl can be fused with cycloalkyl or heterocycle group.

Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by removing “-yl” and adding “-idene” to the name of the corresponding univalent radical, e.g., a naphthyl group with two points of attachment is termed naphthylidene.

The term “heteroaryl”, employed alone or in combination with other terms, refers to a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom selected from N, O and S. Heteroaryl can be fused with cycloalkyl or heterocycle group. In some embodiments, “heteroaryl” refers to
a 5- to 8-membered monocyclic aromatic ring containing one or more, for example, from 1 to 4, or, in some embodiments, from 1 to 3, heteroatoms selected from N, O and S, with the remaining ring atoms being carbon; or
a 8- to 12-membered bicyclic aromatic ring system containing one or more, for example, from 1 to 6, or, in some embodiments, from 1 to 4, or, in some embodiments, from 1 to 3, heteroatoms selected from N, O and S, with the remaining ring atoms being carbon; or
a 11- to 14-membered tricyclic aromatic ring system containing one or more, for example, from 1 to 8, or, in some embodiments, from 1 to 6, or, in some embodiments, from 1 to 4, or in some embodiments, from 1 to 3, heteroatoms selected from N, O and S, with the remaining ring atoms being carbon.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of heteroaryl groups include, but are not limited to, pyrid-2-yl, pyrid-3-yl, pyrid-4-yl, pyrazin-2-yl, pyrazin-3-yl, pyrimidin-2-yl, pyrimidin-4-yl, pyrimidin-5-yl, pyrimidin-6-yl, pyrazol-1-yl, pyrazol-3-yl, pyrazol-4-yl, pyrazol-5-yl, imidazol-1-yl, imidazol-2-yl, imidazol-4-yl, imidazol-5-yl, pyridazinyl, triazinyl, pyrrolyl, oxazolyl, isoxazolyl, thiazolyl, isothiazolyl, thiadiazolyl, triazolyl, tetrazolyl, thienyl, furyl.

Further heteroaryl groups include but are not limited to indolyl, benzothienyl, benzofuryl, benzoimidazolyl, benzotriazolyl, quinoxalinyl, quinolinyl, and isoquinolinyl. “Heteroaryl” is also understood to include the N-oxide derivative of any nitrogen-containing heteroaryl.

Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene.

The term “heterocycle”, employed alone or in combination with other terms, (and variations thereof such as “heterocyclic”, or “heterocyclyl”) broadly refers to a saturated or unsartated mono- or multicyclic (e.g. bicyclic) aliphatic ring system, usually with 3 to 12 ring atoms, wherein at least one (e.g. 2, 3 or 4) ring atom is heteroatom independently selected from O, S, N and P (preferably O, S, N). In a multicyclic heterocycle, two or more rings can be fused or bridged or spiro together. Heterocycle can be fused with aryl or heteroaryl group. In some embodiments, heterocycle is benzocondensed. Heterocycle also includes ring systems substituted with one or more oxo or imino moieties. In some embodiments, the C, N, S and P atoms in the heterocycle ring are optionally substituted by oxo. In some embodiments, the C, S and P atoms in the heterocycle ring are optionally substituted by imino, and imino can be unsubstituted or substituted. The point of the attachment may be carbon atom or heteroatom in the heterocyclic ring, provided that attachment results in the creation of a stable structure. When the heterocyclic ring has substituents, it is understood that the substituents may be attached to any atom in the ring, whether a heteroatom or a carbon atom, provided that a stable chemical structure result.

Suitable heterocycles include, for example, pyrrolidin-1-yl, pyrrolidin-2-yl, pyrrolidin-3-yl, imidazolidin-1-yl, imidazolidin-2-yl, imidazolidin-3-yl, imidazolidin-4-yl, imidazolidin-5-yl, pyrazolidin-1-yl, pyrazolidin-2-yl, pyrazolidin-3-yl, pyrazolidin-4-yl, pyrazolidin-5-yl, piperidin-1-yl, piperidin-2-yl, piperidin-3-yl, piperidin-4-yl, piperazin-1-yl, piperazin-2-yl, piperazin-3-yl, hexahydropyridazin-1-yl, hexahydropyridazin-3-yl and hexahydropyridazin-4-yl. Morpholinyl groups are also contemplated, such as morpholin-1-yl, morpholin-2-yl and morpholin-3-yl. Examples of heterocycle with one or more oxo moieties include but are not limited to, piperidinyl N-oxide, morpholinyl-N-oxide, 1-oxo-thiomorpholinyl and 1,1-dioxo-thiomorpholinyl. Bicyclic heterocycles include, for example:

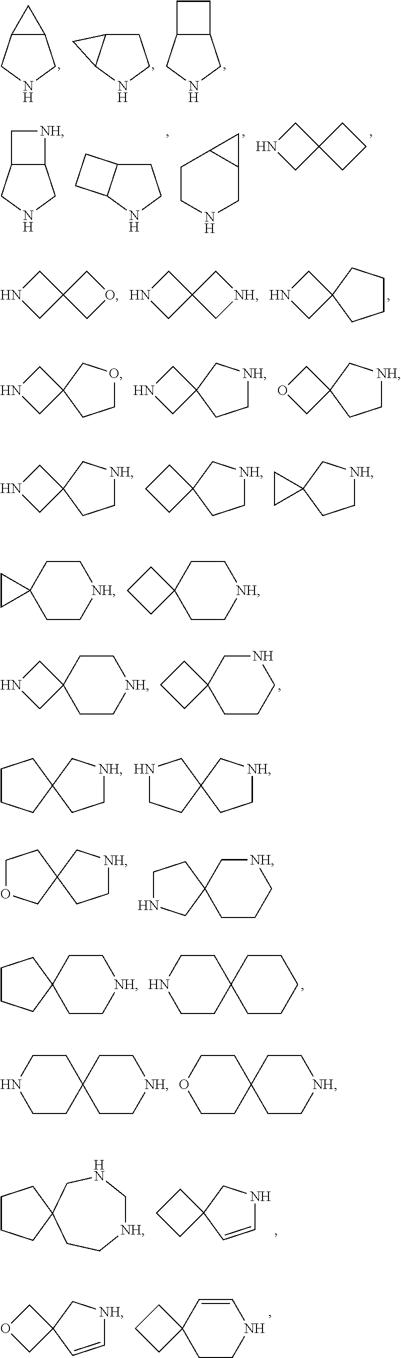

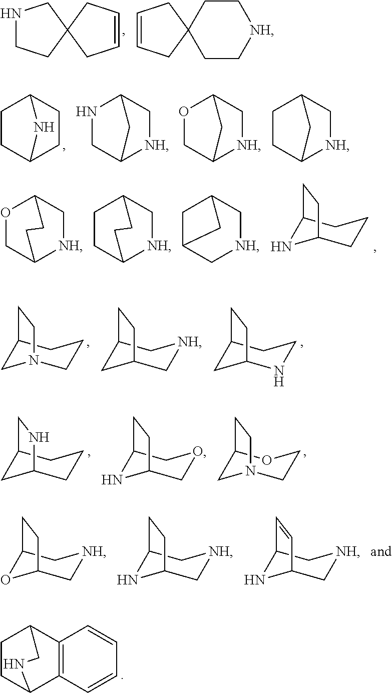

As used herein, “aryl-alkyl” refers to an alkyl moiety as defined above substituted by an aryl group as defined above. Exemplary aryl-alkyl groups include but are not limited to benzyl, phenethyl and naphthylmethyl groups. In some embodiments, aryl-alkyl groups have 7-20 or 7-11 carbon atoms. When used in the phrase “aryl-C₁₋₄ alkyl”, the term “C₁₄” refers to the alkyl portion of the moiety and does not describe the number of atoms in the aryl portion of the moiety.

As used herein, “heterocyclyl-alkyl” refers to alkyl as defined above substituted by heterocyclyl as defined above. When used in the phrase “heterocyclyl-C₁₋₄ alkyl”, the term “C₁₋₄” refers to the alkyl portion of the moiety and does not describe the number of atoms in the heterocyclyl portion of the moiety.

As used herein, “cycloalkyl-alkyl” refers to alkyl as defined above substituted by cycloalkyl as defined above. When used in the phrase “C₃₋₁₀ cycloalkyl-C₁₋₄ alkyl”, the term “C₃₋₁₀” refers to the cycloalkyl portion of the moiety and does not describe the number of atoms in the alkyl portion of the moiety, and the term “C₁₋₄” refers to the alkyl portion of the moiety and does not describe the number of atoms in the cycloalkyl portion of the moiety.

As used herein, “heteroaryl-alkyl” refers to alkyl as defined above substituted by heteroaryl as defined above. When used in the phrase “heteroaryl-C₁₋₄ alkyl”, the term “C₁₋₄” refers to the alkyl portion of the moiety and does not describe the number of atoms in the heteroaryl portion of the moiety.

For avoidance of doubt, reference, for example, to substitution of alkyl, cycloalkyl, heterocyclyl, aryl and/or heteroaryl refers to substitution of each of those groups individually as well as to substitutions of combinations of those groups. That is, if R is aryl-C₁₋₄ alkyl and may be unsubstituted or substituted with at least one substituent, such as one, two, three, or four substituents, independently selected from Rx, it should be understood that the aryl portion may be unsubstituted or substituted with at least one substituent, such as one, two, three, or four substituents, independently selected from R^{X} and the alkyl portion may also be unsubstituted or substituted with at least one substituent, such as one, two, three, or four substituens, independently selected from Rx.

The term “pharmaceutically acceptable salts” refers to salts prepared from pharmaceutically acceptable non-toxic bases or acids including inorganic or organic bases and inorganic or organic acids. Salts derived from inorganic bases may be selected, for example, from aluminum, ammonium, calcium, copper, ferric, ferrous, lithium, magnesium, manganic, manganous, potassium, sodium and zinc salts. Further, for example, the pharmaceutically acceptable salts derived from inorganic bases may be selected from ammonium, calcium, magnesium, potassium and sodium salts. Salts in the solid form may exist in one or more crystalline forms, or polymorphs, and may also be in the form of solvates, such as hydrates. Salts derived from pharmaceutically acceptable organic non-toxic bases may be selected, for example, from salts of primary, secondary and tertiary amines, substituted amines including naturally occurring substituted amines, cyclic amines and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine and tripropylamine, tromethamine.

When the compound disclosed herein is basic, salts may be prepared using at least one pharmaceutically acceptable non-toxic acid, selected from inorganic and organic acids. Such acid may be selected, for example, from acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric and p-toluenesulfonic acids. In some embodiments, such acid may be selected, for example, from citric, hydrobromic, hydrochloric, maleic, phosphoric, sulfuric, fumaric and tartaric acids.

The terms “administration of” and or “administering” a compound or a pharmaceutically acceptable salt should be understood to mean providing a compound or a pharmaceutically acceptable salt thereof to the individual in recognized need of treatment.

The term “effective amount” means the amount of the a compound or a pharmaceutically acceptable salt that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to a pharmaceutical composition is intended to encompass a product comprising the active ingredient (s) and the inert ingredient (s) that make up the carrier, as well as any product which results, directly or indirectly, from combination, complexation or aggregation of any two or more of the ingredients, or from dissociation of one or more of the ingredients, or from other types of reactions or interactions of one or more of the ingredients.

The term “pharmaceutically acceptable” it is meant compatible with the other ingredients of the formulation and not unacceptably deleterious to the recipient thereof.

The term “subject” as used herein in reference to individuals suffering from a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “protecting group” or “Pg” refers to a substituent that can be commonly employed to block or protect a certain functionality while reacting other functional groups on the compound. For example, an “amino-protecting group” is a substituent attached to an amino group that blocks or protects the amino functionality in the compound. Suitable amino-protecting groups include but are not limited to acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include but are not limited to acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The term “NH protecting group” as used herein includes, but not limited to, trichloroethoxycarbonyl, tribromoethoxycarbonyl, benzyloxycarbonyl, para-nitrobenzylcarbonyl, ortho-bromobenzyloxycarbonyl, chloroacetyl, dichloroacetyl, trichloroacetyl, trifluoroacetyl, phenylacetyl, formyl, acetyl, benzoyl, tert-amyloxycarbonyl, tert-butoxycarbonyl, para-methoxybenzyloxycarbonyl, 3,4-dimethoxybenzyl-oxycarbonyl, 4-(phenylazo)-benzyloxycarbonyl, 2-furfuryloxycarbonyl, diphenylmethoxycarbonyl, 1,1-dimethylpropoxy-carbonyl, isopropoxycarbonyl, phthaloyl, succinyl, alanyl, leucyl, 1-adamantyloxycarbonyl, 8-quinolyloxycarbonyl, benzyl, diphenylmethyl, triphenylmethyl, 2-nitrophenylthio, methanesulfonyl, para-toluenesulfonyl, N,N-dimethylaminomethylene, benzylidene, 2-hydroxybenzylidene, 2-hydroxy-5-chlorobenzylidene, 2-hydroxy-1-naphthylmethylene, 3-hydroxy-4-pyridylmethylene, cyclohexylidene, 2-ethoxycarbonylcyclohexylidene, 2-ethoxycarbonylcyclopentylidene, 2-acetylcyclohexylidene, 3,3-dimethyl-5-oxycyclo-hexylidene, diphenylphosphoryl, dibenzylphosphoryl, 5-methyl-2-oxo-2H-1,3-dioxol-4-yl-methyl, trimethylsilyl, triethylsilyl and triphenylsilyl.

The term “C(O)OH protecting group” as used herein includes, but not limited to, methyl, ethyl, n-propyl, isopropyl, 1,1-dimethylpropyl, n-butyl, tert-butyl, phenyl, naphthyl, benzyl, diphenylmethyl, triphenylmethyl, para-nitrobenzyl, para-methoxybenzyl, bis(para-methoxyphenyl)methyl, acetylmethyl, benzoylmethyl, para-nitrobenzoylmethyl, para-bromobenzoylmethyl, para-methanesulfonylbenzoylmethyl, 2-tetrahydropyranyl, 2-tetrahydrofuranyl, 2,2,2-trichloro-ethyl, 2-(trimethylsilyl)ethyl, acetoxymethyl, propionyloxymethyl, pivaloyloxymethyl, phthalimidomethyl, succinimidomethyl, cyclopropyl, cyclobutyl, cyclopentyl, cyclohexyl, methoxymethyl, methoxyethoxymethyl, 2-(trimethylsilyl)ethoxymethyl, benzyloxymethyl, methylthiomethyl, 2-methylthioethyl, phenylthiomethyl, 1,1-dimethyl-2-propenyl, 3-methyl-3-butenyl, allyl, trimethylsilyl, triethylsilyl, triisopropylsilyl, diethylisopropylsilyl, tert-butyldimethylsilyl, tert-butyldiphenylsilyl, diphenylmethylsilyl and tert-butylmethoxyphenylsilyl.

The term “OH or SH protecting group” as used herein includes, but not limited to, benzyloxycarbonyl, 4-nitrobenzyloxycarbonyl, 4-bromobenzyloxycarbonyl, 4-methoxybenzyloxycarbonyl, 3,4-dimethoxybenzyloxycarbonyl, methoxycarbonyl, ethoxycarbonyl, tert-butoxycarbonyl, 1,1-dimethylpropoxycarbonyl, isopropoxycarbonyl, isobutyloxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, 2-(triphenylphosphonio)ethoxycarbonyl, 2-furfuryloxycarbonyl, 1-adamantyloxycarbonyl, vinyloxycarbonyl, allyloxycarbonyl, 4-ethoxy-1-naphthyloxycarbonyl, 8-quinolyloxycarbonyl, acetyl, formyl, chloroacetyl, dichloroacetyl, trichloroacetyl, trifluoroacetyl, methoxyacetyl, phenoxyacetyl, pivaloyl, benzoyl, methyl, tert-butyl, 2,2,2-trichloroethyl, 2-trimethylsilylethyl, 1,1-dimethyl-2-propenyl, 3-methyl-3-butenyl, allyl, benzyl (phenylmethyl), para-methoxybenzyl, 3,4-dimethoxybenzyl, diphenylmethyl, triphenylmethyl, tetrahydrofuryl, tetrahydropyranyl, tetrahydrothiopyranyl, methoxymethyl, methylthiomethyl, benzyloxymethyl, 2-methoxyethoxymethyl, 2,2,2-trichloro-ethoxymethyl, 2-(trimethylsilyl)ethoxymethyl, 1-ethoxyethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl and tert-butylmethoxyphenylsilyl.

Geometric isomers may exist in the present compounds. Compounds of this invention may contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers. Substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantane ring system are designated as being of Z or E relative configuration. For examples, see C. D. Jones, M. Kaselj, R. N. Salvatore, W. J. le Noble J. Org. Chem. 1998, 63, 2758-2760.

Compounds of this invention may contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration present in the higher amount, preferably an excess of about 85-90%, more preferably an excess of about 95-99%, and still more preferably an excess greater than about 99%. Accordingly, this invention includes racemic mixtures, relative and absolute stereoisomers, and mixtures of relative and absolute stereoisomers.

Isotope Enriched or Labeled Compounds.

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, nitrogen, oxygen, phosphorous, sulfur, fluorine, chlorine and iodine include, but are not limited to, ²H, ³H, ¹³C, ¹⁴C, ¹⁵N, ¹⁸O, ³²P, ³⁵S, ¹⁸F, ³⁶Cl and ¹²⁵I. Compounds that contain other isotopes of these and/or other atoms are within the scope of this invention.

In another embodiment, the isotope-labeled compounds contain deuterium (²H), tritium (3H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuterated acid such as D₂SO₄/D₂O.

The isotope-labeled compounds of the invention may be used as standards to determine the effectiveness of Bcl-2 inhibitors in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al, J. Labelled Comp. Radiopharmaceut., 36(10):927-932 (1995); Kushner et al., Can. J. Physiol. Pharmacol, 77, 79-88 (1999).

In addition, non-radioactive isotope containing drugs, such as deuterated drugs called “heavy drugs” can be used for the treatment of diseases and conditions related to Bcl-2 activity. Increasing the amount of an isotope present in a compound above its natural abundance is called enrichment. Examples of the amount of enrichment include but are not limited to from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

In an Embodiment (1), disclosed herein is a compound of formula (I):

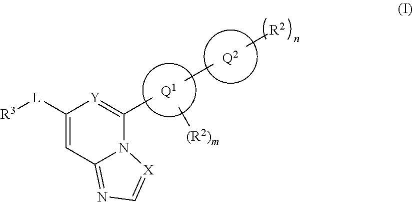

or a pharmaceutically acceptable salt thereof, wherein:

Q¹ is selected from aryl and heteroaryl;

Q² is heterocyclyl;

X is selected from CR⁴ and N;

Y is selected from CR⁵ and N;

L is selected from a bond, —(CR^{C0}R^{D0})ᵤ—, —(CR^{C0}R^{D0})ᵤO(CR^{C0}R^{D0})ₜ—, —(CR^{C0}R^{D0})ᵤNR^{A0}(CR^{C0}R^{D0})ₜ—, —(CR^{C0}R^{D0})ᵤS(CR^{C0}R^{D0})ₜ—, —(CR^{C0}R^{D0})ᵤC(O)NR^{A0}(CR^{C0}R^{D0})ₜ—, —(CR^{C0}R^{D0})ᵤNR^{A0}C(O)(CR^{C0}R^{D0})ₜ—, —(CR^{C0}R^{D0})ᵤNR^{A0}C(O)NR^{B0}(CR^{C0}R^{D0})ₜ—, —(CR^{C0}R^{D0})ᵤS(O)ᵣ(CR^{C0}R^{D0})ₜ—, —(CR^{C0}R^{D0})ᵤS(O)ᵣNR^{A0}(CR^{C0}R^{D0})ₜ—, —(CR^{C0}R^{D0})ᵤNR^{A0}S(O)ᵣ(CR^{C0}R^{D0})ₜ—, and —(CR^{C0}R^{D0})ᵤNR^{A0}S(O)ᵣNR^{B0}(CR^{C0}R^{D0})ₜ—, each R¹ is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═NR^{E1})R^{A1}, —C(═N—OR^{B1})R^{A1}, —C(O)OR^{A1}, —OC(O)R^{A1}, —C(O)NR^{A1}R^{B1}, —NR^{A1}C(O)R^{B1}, —C(═NR^{E1})NR^{A1}R^{B1}, —NR^{A1}C(═NR^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1})NR^{A1}R^{B1}, —S(O)ᵣR^{A1}, —S(O)(═NR^{E1})R^{B1}, —N═S(O)R^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}, —S(O)ᵣNR^{A1}R^{B1}, —S(O)(═NR^{E1})NR^{A1}R^{B1}, —NR^{A1}S(O)₂NR^{A1}R^{B1}, —NR^{A1}S(O)(═NR^{E1})NR^{A1}R^{B1}, —P(O)R^{A1}R^{B1} and —P(O)(OR^{A1})(OR^{B1}), wherein alkyl, alkenyl, alkynyl, cycloalkyl, heterocyclyl, aryl and heteroaryl are each unsubstituted or substituted with at least one substituent, independently selected from R^{X};

each R² is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —NR^{A2}C(O)R^{B2}, —C(═NR^{E2})NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2})NR^{A2}R^{B2}, —S(O)ᵣR^{A2}, —S(O)(═NR^{E2})R^{B2}, —N═S(O)R^{A2}R^{B2}, —S(O)₂OR^{A2}, —OS(O)₂R^{A2}, —NR^{A2}S(O)ᵣR^{B2}, —NR^{A2}S(O)(═NR^{E2})R^{B2}, —S(O)ᵣNR^{A2}R^{B2}, —S(O)(═NR^{E2})NR^{A2}R^{B2}, —NR^{A2}S(O)₂NR^{A2}R^{B2}, —NR^{A2}S(O)(═NR^{E2})NR^{A2}R^{B2}, —P(O)R^{A2}R^{B2} and —P(O)(OR^{A2})(OR^{B2}), wherein alkyl, alkenyl, alkynyl, cycloalkyl, heterocyclyl, aryl and heteroaryl are each unsubstituted or substituted with at least one substituent, independently selected from R^{X};

R³ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —NR^{A3}C(O)R^{B3}, —C(═NR^{E3})NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3})NR^{A3}R^{B3}, —S(O)ᵣR^{A3}, —S(O)(═NR^{E3})R^{B3}, —N═S(O)R^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3})NR^{A3}R^{B3}, —NR^{A3}S(O)₂NR^{A3}R^{B3}, —NR^{A3}S(O)(═NR^{E3})NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R⁶;

R⁴ and R⁵ are independently selected from hydrogen, halogen, CN, C₁₋₁₀ alkyl and C₃₋₁₀ cycloalkyl, wherein alkyl and cycloalkyl are unsubstituted or substituted with at least one substituent, independently selected from R^{X};

R⁶ is selected from hydrogen, halogen, OH, C₁₋₁₀ alkyl, C₃₋₁₀ cycloalkyl, C₃₋₁₀ cycloalkyl-C₁₋₄ alkyl, heterocyclyl and heterocyclyl-C₁₋₄ alkyl, wherein alkyl, cycloalkyl and heterocyclyl are unsubstituted or substituted with at least one substituent, independently selected from R^{X};

each R^{A0}, R^{A1}, R^{A2}, R^{A3}, R^{B0}, R^{B}1, R^{B2} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X};

or each “R^{A0} and R^{B0}”, “R^{A1} and R^{B1}”, “R^{A2} and R^{B2}” or “A³ and R^{B3}” together with the atom(s) to which they are attached form a heterocyclic ring of 4 to 12 members containing 0, 1 or 2 additional heteroatoms independently selected from oxygen, sulfur, nitrogen and phosphorus and optionally substituted with 1, 2 or 3 R^{X} groups;

each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{X};

or R^{C0} and R^{D0} together with the carbon atom(s) to which they are attached form a ring of 3 to 12 members containing 0, 1 or 2 heteroatoms independently selected from oxygen, sulfur and nitrogen and optionally substituted with 1 2 or 3 R^{X} groups;

each R^{E1}, R^{E2} and R^{E3} are independently selected from hydrogen, C₁₋₁₀ alkyl, CN, NO₂, —OR^{a1}, —SR^{a1}, —S(O)ᵣR^{a1}, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from R^{X};

each R^{X} is independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1}, —(CR^{c1}R^{d1})ₜC(═N—OR^{b1})R^{a1}, —(CR^{c1}R^{d1})ₜC(O)OR^{b1}, —(CR^{c1}R^{d1})ₜOC(O)R^{b1}, —(CR^{c1}R^{d1})ₜC(O)NR^{a1}R^{b1}, —(CR^{c1}R^{d1})ₜNR^{a1}C(O)R^{b1}, —(CR^{c1}R^{d1})ₜC(═NR^{e1})NR^{a1}R^{b1}, —(CR^{c1}R^{d1})ₜNR^{a1}C(═NR^{e1})R^{b1}, —(CR^{c1}R^{d1})ₜOC(O)NR^{a1}R^{b1}, —(CR^{c1}R^{d1})ₜNR^{a1}C(O)OR^{b1}, —(CR^{c1}R^{d1})ₜNR^{a1}C(O)NR^{a1}R^{b1}, —(CR^{c1}R^{d1})ₜNR^{a1}C(S)NR^{a1}R^{b1}, —(CR^{c1}R^{d1})ₜNR^{a1}C(═NR^{e1})NR^{a1}R^{b1}, —(CR^{c1}R^{d1})ₜS(O)ᵣR^{b1}, —(CR^{c1}R^{d1})ₜS(O)(═NR^{e1})R^{b1}, —(CR^{c1}R^{d1})ₜN═S(O)R^{a1}R^{b1}, —(CR^{c1}R^{d1})ₜS(O)₂OR^{b1}, —(CR^{c1}R^{d1})ₜOS(O)₂R^{b1}, —(CR^{c}R^{d1})ₜNR^{a1}S(O)ᵣR^{b1}, —(CR^{c1}R^{d1})ₜNR^{a1}S(O)(═NR^{e1})R^{b1}, —(CR^{c1}R^{d1})ₜS(O)ᵣNR^{a1}R^{b1}, —(CR^{c1}R^{d1})ₜS(O)(═NR^{e1})NR^{a1}R^{b1}, —(CR^{c1}R^{d1})ₜNR^{a1}S(O)₂NR^{a1}R^{b1}, —(CR^{c1}R^{d1})ₜNR^{a1}S(O)(═NR^{e1})NR^{a1}R^{b1}, —(CR^{c1}R^{d1})ₜP(O)R^{a1}R^{b1} and —(CR^{c1}R^{d1})ₜP(O)(OR^{a1})(OR^{b1}), wherein alkyl, alkenyl, alkynyl, cycloalkyl, heterocyclyl, aryl and heteroaryl are each unsubstituted or substituted with at least one substituent, independently selected from R^{Y};

each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};

or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{Y} groups; each R^{e1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};

or R^{c1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 R^{Y} groups;

each R^{e1} is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR^{a2}R^{b2} and —C(O)NR^{a2}R^{b2};

each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})ₜNR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a2}, —(CR^{c2}R^{d2})ₜC(═N—OR^{b2})R^{a2}, —(CR^{c2}R^{d2})ₜC(O)OR^{b2}, —(CR^{c2}R^{d2})ₜOC(O)R^{b2}, —(CR^{c2}R^{d2})ₜC(O)NR^{a2}R^{b2}, —(CR^{c2}R^{d2})ᵣNR^{a2}C(O)R^{b2}, —(CR^{c2}R^{d2})ᵣC(═NR^{e2})NR^{a2}R^{b2}, —(CR^{c2}R^{d2})ₜNR^{a2}C(═NR^{e2})R^{b2}, —(CR^{c2}R^{d2})ₜOC(O)NR^{a2}R^{b2}, —(CR^{c2}R^{d2})ᵣNR^{a2}C(O)OR^{b2}, —(CR^{c2}R^{d2})ᵣNR^{a2}C(O)NR^{a2}R^{b2}, —(CR^{c2}R^{d2})ᵣNR^{a2}C(S)NR^{a2}R^{b2}, —(CR^{c2}R^{d2})ᵣNR^{a2}C(═NR^{e2})NR^{a2}R^{b2}, —(CR^{c2}R^{d2})ₜS(O)ᵣR^{b2}, —(CR^{c2}R^{d2})ᵣS(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2})ₜOS(O)₂R^{b2}, —(CR^{c2}R^{d2})ₜNR^{a2}S(O)ᵣR^{b2}, —(CR^{c2}R^{d2})ᵣNR^{a2}S(O)(═NR^{e2})R^{b2}, —(CR^{c2}R^{d2})ₜS(O)ᵣNR^{a2}R^{b2}, —(CR^{c2}R^{d2})ᵣS(O)(═NR^{e2})NR^{a2}R^{b2}, —(CR^{c2}R^{d2})ᵣNR^{a2}S(O)₂NR^{a2}R^{b2}, —(CR^{c2}R^{d2})ᵣNR^{a2}S(O)(═NR^{e2})NR^{a2}R^{b2}, —(CR^{e2}R^{d2})ᵣP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;

each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

or R^{a2} and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

each R^{e2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

or R^{c2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;

each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O)C₁₋₄ alkyl, —C(O)C₃₋₁₀ cycloalkyl, —C(O)OC₁_₄ alkyl, —C(O)OC₃₋₁₀ cycloalkyl, —C(O)N(C₁₋₄ alkyl)₂, —C(O)N(C₃₋₁₀ cycloalkyl)₂, —S(O)₂C₁₋₄ alkyl, —S(O)₂C₃₋₁₀ cycloalkyl, —S(O)₂N(C₁₋₄ alkyl)₂ and —S(O)₂N(C₃₋₁₀ cycloalkyl)₂; m is selected from 1, 2 and 3;

n is selected from 1, 2 and 3;

each r is independently selected from 0, 1 and 2;

each t is independently selected from 0, 1, 2, 3 and 4;

each u is independently selected from 0, 1, 2, 3 and 4.

In another Embodiment (2), the invention provides a compound of Embodiment (1) or a pharmaceutically acceptable salt thereof, wherein L is a bond, R³ is pyrazolyl, and the compound has the formula (II),

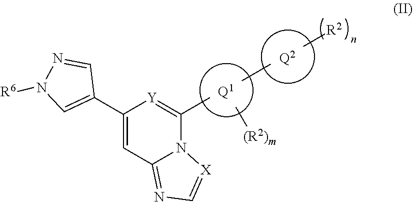

wherein Q¹, Q², X, Y, R¹, R², R⁶, n and m are as defined in formula (I).

In another Embodiment (3), the invention provides a compound of Embodiment (1) or a pharmaceutically acceptable salt thereof, wherein L is O, and the compound has the formula (III),

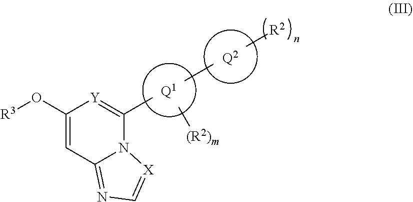

wherein Q¹, Q², X, Y, R¹, R², R³, n and m are as defined in formula (I).

In another Embodiment (4), the invention provides a compound of Embodiment (2) or a pharmaceutically acceptable salt thereof, wherein Y is CH, and the compound has the formula (IV),

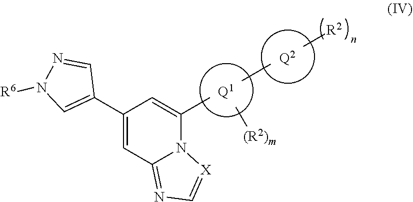

wherein Q¹, Q², X, R¹, R², R⁶, n and m are as defined in formula (I).

In another Embodiment (5), the invention provides a compound of Embodiment (2) or a pharmaceutically acceptable salt thereof, wherein Y is N, and the compound has the formula (V),

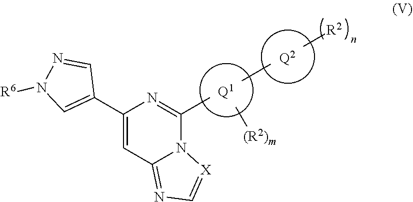

wherein Q¹, Q², X, R¹, R², R⁶, n and m are as defined in formula (I).

In another Embodiment (6), the invention provides a compound of any one of Embodiment (2), (4)-(5) or a pharmaceutically acceptable salt thereof, wherein R⁶ is C₁₋₁₀ alkyl, wherein alkyl is unsubstituted or substituted with at least one substituent independently selected from R^{X}.

In another Embodiment (7), the invention provides a compound of Embodiment (6) or a pharmaceutically acceptable salt thereof, wherein R⁶ is selected from methyl,

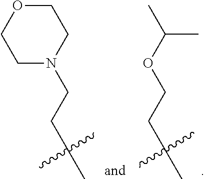

In another Embodiment (8), the invention provides a compound of Embodiment (3) or a pharmaceutically acceptable salt thereof, wherein R³ is selected from C₁₋₁₀ alkyl, wherein alkyl is unsubstituted or substituted with at least one substituent, independently selected from R⁶.

In another Embodiment (9), the invention provides a compound of Embodiment (8) or a pharmaceutically acceptable salt thereof, wherein R³ is selected from methyl and ethyl, and methyl and ethyl are each unsubstituted or substituted with at least one substituent, independently selected from R⁶, and R⁶ is selected from C₁₋₁₀ alkyl, C₃₋₁₀ cycloalkyl and OH, wherein alkyl and cycloalkyl is unsubstituted or substituted with at least one substituent, independently selected from R^{X}.

In another Embodiment (10), the invention provides a compound of Embodiment (9) or a pharmaceutically acceptable salt thereof, wherein R⁶ is selected from methyl, cyclopropyl and OH, and R^{X} is selected from —(CR^{c1}R^{d1})ₜN═S(O)R^{a}R^{b1}, halogen and OH.

In another Embodiment (11), the invention provides a compound of Embodiment (10) or a pharmaceutically acceptable salt thereof, wherein R^{X} is selected from

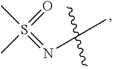 [SMILES: *N=S(C)(C)=O]

### F and OH.

In another Embodiment (12), the invention provides a compound of any one of Embodiment (1)-(11) or a pharmaceutically acceptable salt thereof, wherein Q¹ is selected from pyridinyl, pyrimidyl, pyrazinyl and phenyl.

In another Embodiment (13), the invention provides a compound of Embodiment (12) or a pharmaceutically acceptable salt thereof, wherein Q¹ is pyridinyl.

In another Embodiment (14), the invention provides a compound of any one of Embodiment (1)-(13) or a pharmaceutically acceptable salt thereof, wherein X is CR⁴.

In another Embodiment (15), the invention provides a compound of Embodiment (14) or a pharmaceutically acceptable salt thereof, wherein R⁴ is CN.

In another Embodiment (16), the invention provides a compound of any one of Embodiment (1)-(13) or a pharmaceutically acceptable salt thereof, wherein X is N.

In another Embodiment (17), the invention provides a compound of any one of Embodiment (1)-(16) or a pharmaceutically acceptable salt thereof, wherein Q² is 4-7 membered heterocyclyl.

In another Embodiment (18), the invention provides a compound of Embodiment (17) or a pharmaceutically acceptable salt thereof, wherein Q² is selected from

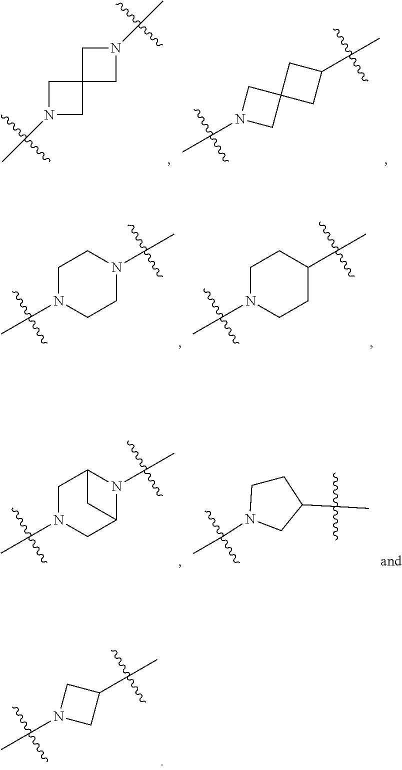

In another Embodiment (19), the invention provides a compound of any one of Embodiment (1)-(18) or a pharmaceutically acceptable salt thereof, wherein R¹ is selected from hydrogen and halogen. In another Embodiment, R¹ is selected from Br and C₁. In another Embodiment, R¹ is selected from hydrogen.

In another Embodiment (20), the invention provides a compound of any one of Embodiment (1)-(19) or a pharmaceutically acceptable salt thereof, wherein R² is selected from hydrogen, C₁₋₁₀ alkyl, aryl-C₁₋₄ alkyl, heteroaryl-C₁₋₄ alkyl, —OR^{A2}, —C(O)R^{A2}, —C(O)OR^{A2} and —C(O)NR^{A2}R^{B2}, wherein alkyl, aryl and heteroaryl are each unsubstituted or substituted with at least one substituent, independently selected from Rx.

In another Embodiment (21), the invention provides a compound of Embodiment (20) or a pharmaceutically acceptable salt thereof, wherein R² is selected from hydrogen, ethyl, benzyl, pyridinylmethyl, Boc, —OR^{A2}, —C(O)R^{A2}, —C(O)NR^{A2}R^{B2},

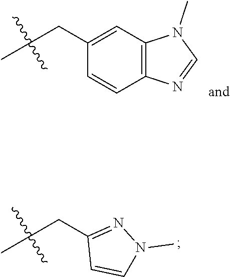

for example, the ethyl, benzyl, pyridinylmethyl, Boc, —OR^{A2}, —C(O)R^{A2}, —C(O)NR^{A2}R^{B2},

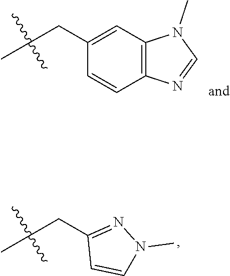

particularly the ethyl, benzyl, pyridinylmethyl,

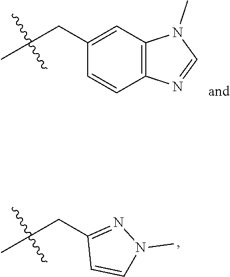

are each unsubstituted or substituted with at least one substituent, independently selected from R^{X}.

In another Embodiment (22), the invention provides a compound of Embodiment (21) or a pharmaceutically acceptable salt thereof, wherein the substituent R^{X} of ethyl, benzyl, pyridinylmethyl,

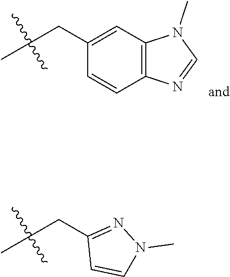

and are independently selected from halogen, C₁₋₁₀ alkyl, —(CR^{c1}R^{d1})ₜNR^{a1}R^{b1}, —(CR^{c1}R^{a1})ₜS(O)ᵣR^{b} and —(CR^{c1}R^{d1})ₜOR^{b1}.

In another Embodiment (23), the invention provides a compound of Embodiment (22) or a pharmaceutically acceptable salt thereof, wherein R^{X} is independently selected from halogen, methyl, methoxy, dimethylamino,

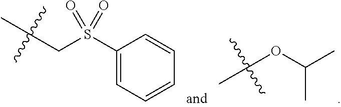

In another Embodiment (24), the invention provides a compound of any one of Embodiment (20)-(21) or a pharmaceutically acceptable salt thereof, wherein R^{A2} is selected from hydrogen, C₁₋₁₀ alkyl, aryl, aryl-C₁₋₄ alkyl, heteroaryl and heteroaryl-C₁₋₄ alkyl, wherein the alkyl, aryl and heteroaryl in R^{A2} are each unsubstituted or substituted with at least one substituent independently selected from R^{X}.

In another Embodiment (25), the invention provides a compound of Embodiment (24) or a pharmaceutically acceptable salt thereof, wherein R^{A2} is selected from hydrogen, methyl, butyl, pentyl, pyridinyl, phenyl, pyridinylmethyl and pyridazinyl, and the substituent R^{X} of R^{A2} is independently selected from halogen, C₁₋₁₀ alkyl, cyclopropyl, ethynyl, vinyl, —OH, methoxy, ethoxy, dimethylamino, aminomethyl, phenyl, benzyl, and

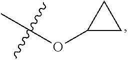 [SMILES: *OC1CC1]

wherein alkyl, phenyl and benzyl are each unsubstituted or substituted with at least one substituent independently selected from R^{Y}.

In another Embodiment (26), the invention provides a compound of any one of Embodiment (20)-(25) or a pharmaceutically acceptable salt thereof, wherein R² is selected from hydrogen, methyl, ethyl, —OH, Boc,

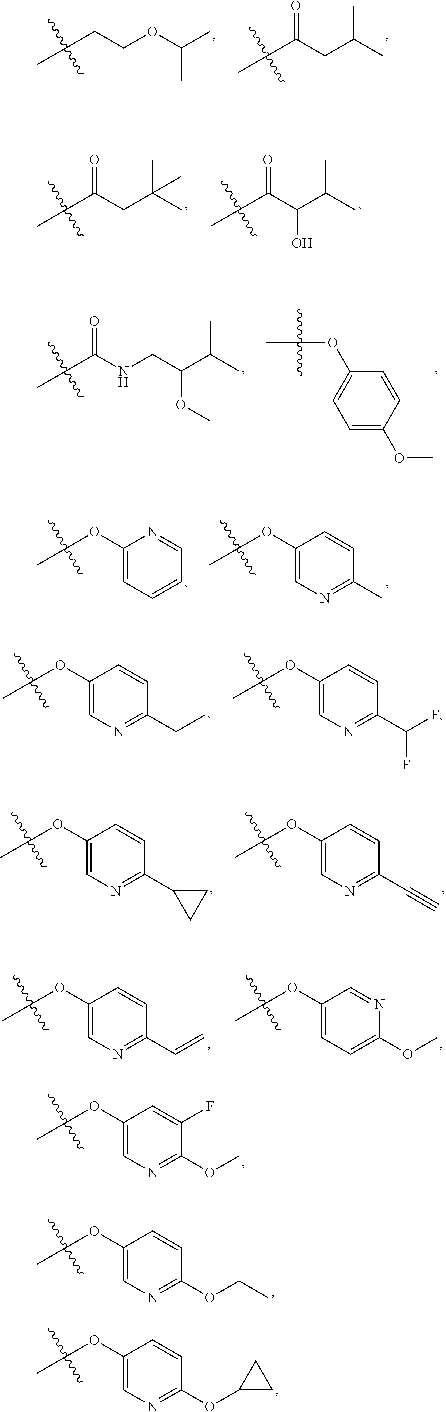

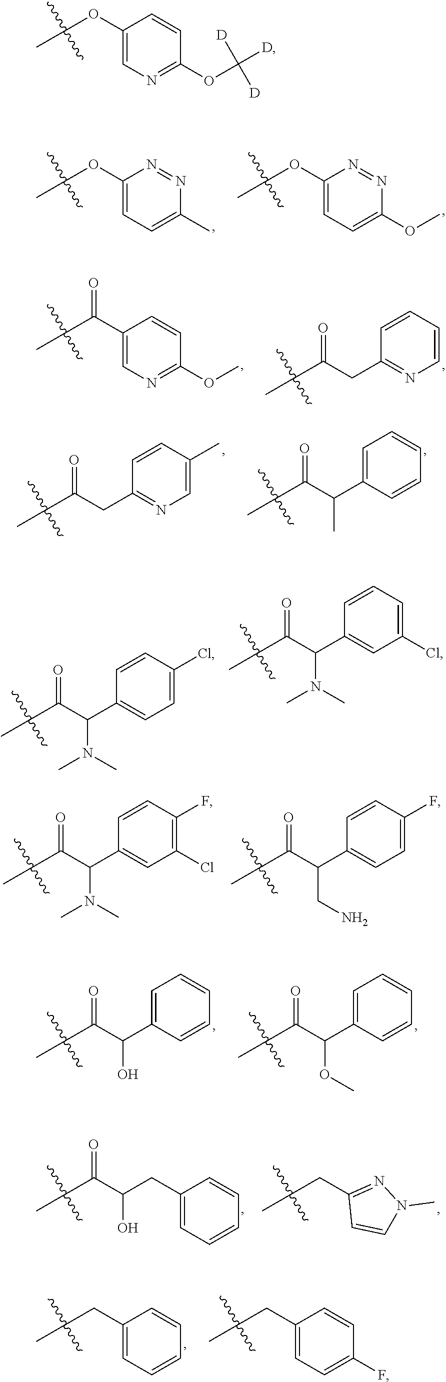

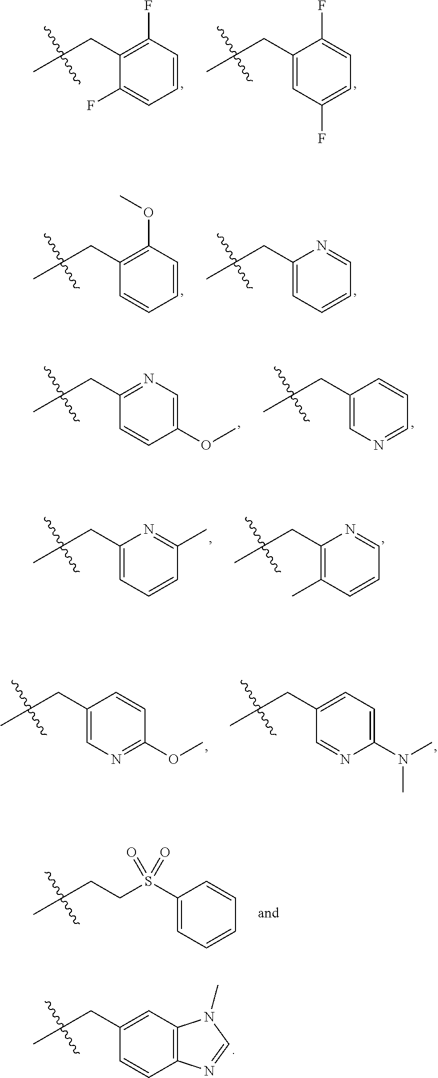

In another Embodiment (27), the invention provides a compound selected from

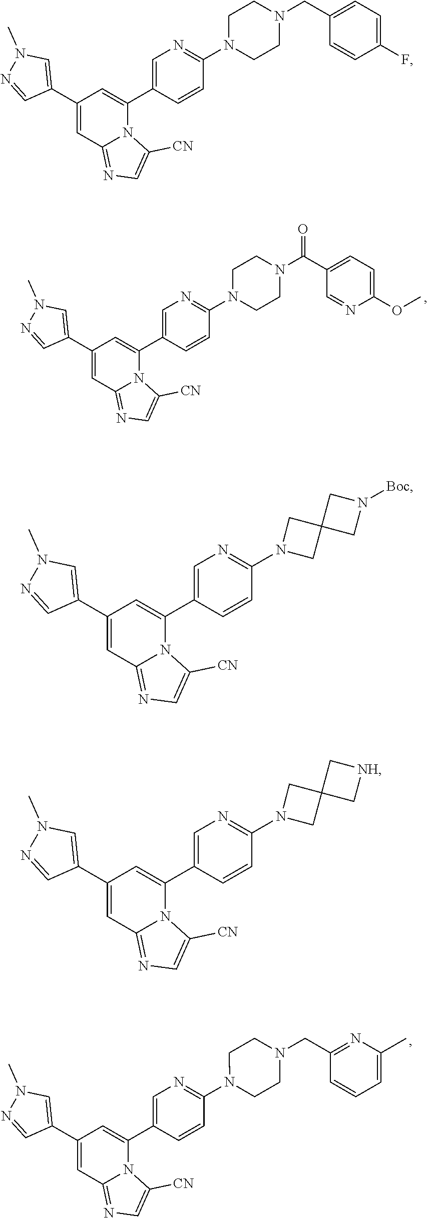 [SMILES: Cn1cc(-c2cc(-c3ccc(N4CCN(Cc5ccc(F)cc5)CC4)nc3)n3c(C#N)cnc3c2)cn1 ; COc1ccc(C(=O)N2CCN(c3ccc(-c4cc(-c5cnn(C)c5)cc5ncc(C#N)n45)cn3)CC2)cn1 ; Cn1cc(-c2cc(-c3ccc(N4CC5(CN(C(=O)OC(C)(C)C)C5)C4)nc3)n3c(C#N)cnc3c2)cn1 ; Cn1cc(-c2cc(-c3ccc(N4CC5(CNC5)C4)nc3)n3c(C#N)cnc3c2)cn1 ; Cc1cccc(CN2CCN(c3ccc(-c4cc(-c5cnn(C)c5)cc5ncc(C#N)n45)cn3)CC2)n1]

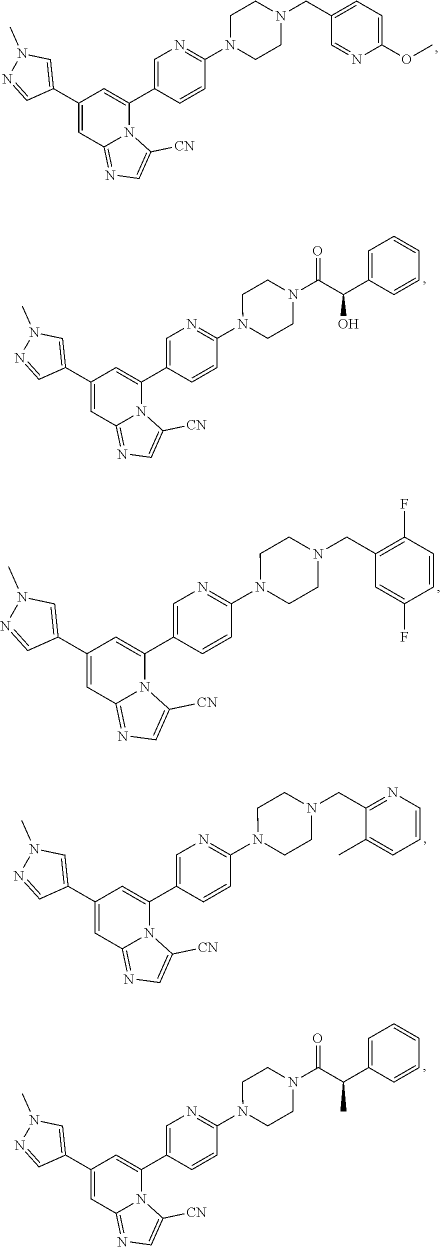 [SMILES: COc1ccc(CN2CCN(c3ccc(-c4cc(-c5cnn(C)c5)cc5ncc(C#N)n45)cn3)CC2)cn1 ; Cn1cc(-c2cc(-c3ccc(N4CCN(C(=O)[C@H](O)c5ccccc5)CC4)nc3)n3c(C#N)cnc3c2)cn1 ; Cn1cc(-c2cc(-c3ccc(N4CCN(Cc5cc(F)ccc5F)CC4)nc3)n3c(C#N)cnc3c2)cn1 ; Cc1cccnc1CN1CCN(c2ccc(-c3cc(-c4cnn(C)c4)cc4ncc(C#N)n34)cn2)CC1 ; C[C@@H](C(=O)N1CCN(c2ccc(-c3cc(-c4cnn(C)c4)cc4ncc(C#N)n34)cn2)CC1)c1ccccc1]

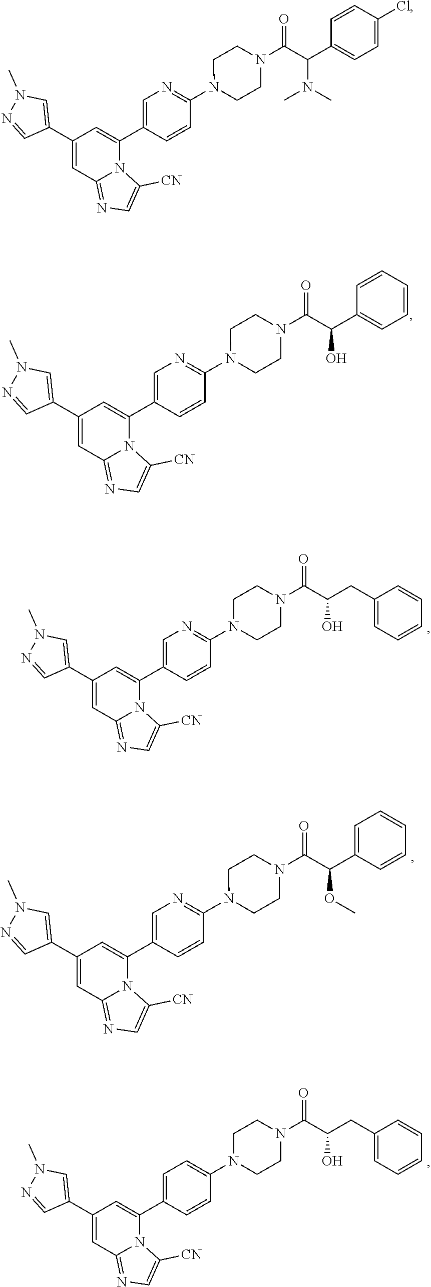 [SMILES: CN(C)C(C(=O)N1CCN(c2ccc(-c3cc(-c4cnn(C)c4)cc4ncc(C#N)n34)cn2)CC1)c1ccc(Cl)cc1 ; Cn1cc(-c2cc(-c3ccc(N4CCN(C(=O)[C@H](O)c5ccccc5)CC4)nc3)n3c(C#N)cnc3c2)cn1 ; Cn1cc(-c2cc(-c3ccc(N4CCN(C(=O)[C@@H](O)Cc5ccccc5)CC4)nc3)n3c(C#N)cnc3c2)cn1 ; CO[C@@H](C(=O)N1CCN(c2ccc(-c3cc(-c4cnn(C)c4)cc4ncc(C#N)n34)cn2)CC1)c1ccccc1 ; Cn1cc(-c2cc(-c3ccc(N4CCN(C(=O)[C@@H](O)Cc5ccccc5)CC4)cc3)n3c(C#N)cnc3c2)cn1]

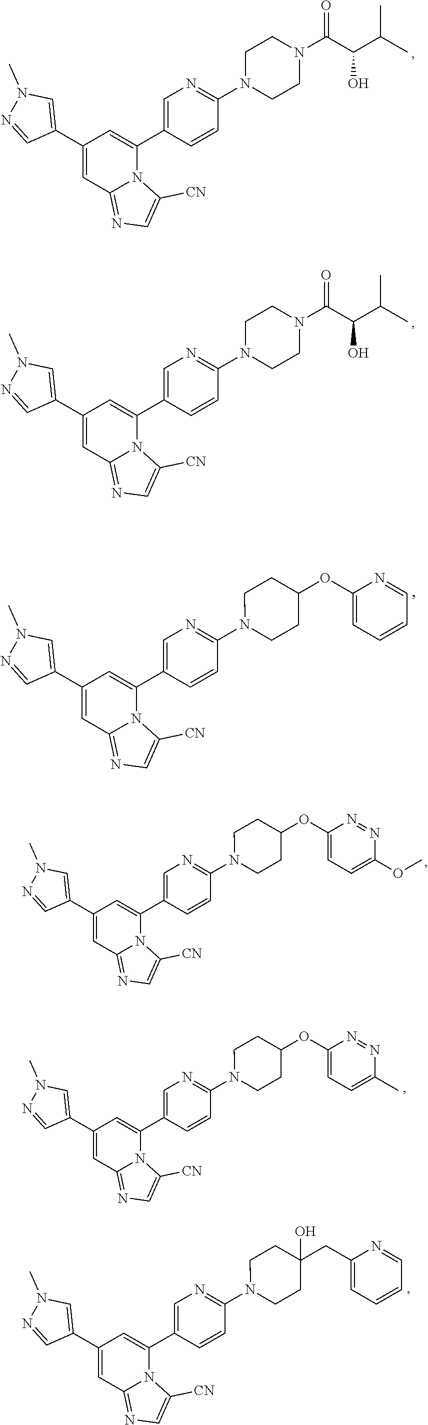 [SMILES: CC(C)[C@H](O)C(=O)N1CCN(c2ccc(-c3cc(-c4cnn(C)c4)cc4ncc(C#N)n34)cn2)CC1 ; CC(C)[C@@H](O)C(=O)N1CCN(c2ccc(-c3cc(-c4cnn(C)c4)cc4ncc(C#N)n34)cn2)CC1 ; Cn1cc(-c2cc(-c3ccc(N4CCC(Oc5ccccn5)CC4)nc3)n3c(C#N)cnc3c2)cn1 ; COc1ccc(OC2CCN(c3ccc(-c4cc(-c5cnn(C)c5)cc5ncc(C#N)n45)cn3)CC2)nn1 ; Cc1ccc(OC2CCN(c3ccc(-c4cc(-c5cnn(C)c5)cc5ncc(C#N)n45)cn3)CC2)nn1 ; Cn1cc(-c2cc(-c3ccc(N4CCC(O)(Cc5ccccn5)CC4)nc3)n3c(C#N)cnc3c2)cn1]

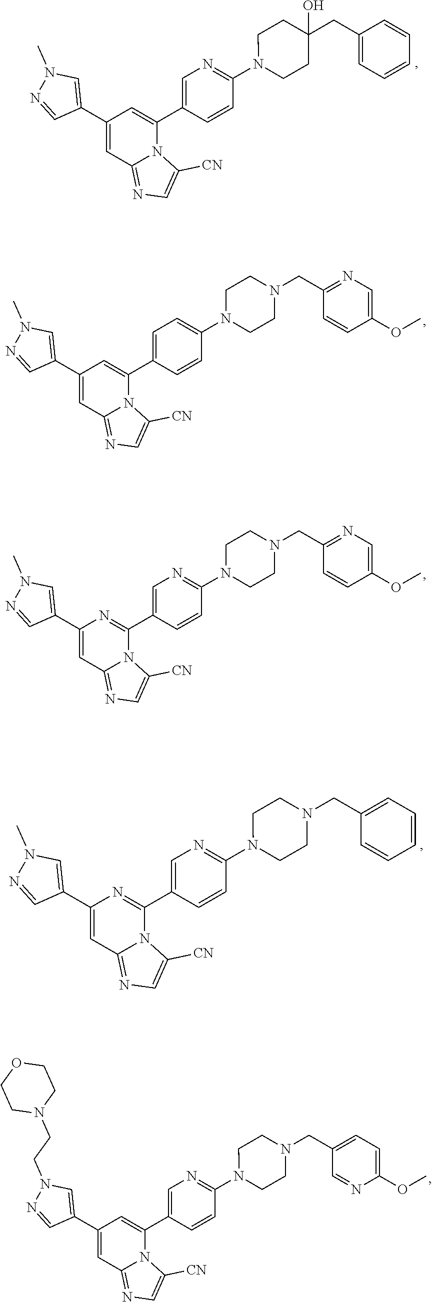 [SMILES: Cn1cc(-c2cc(-c3ccc(N4CCC(O)(Cc5ccccc5)CC4)nc3)n3c(C#N)cnc3c2)cn1 ; COc1ccc(CN2CCN(c3ccc(-c4cc(-c5cnn(C)c5)cc5ncc(C#N)n45)cc3)CC2)nc1 ; COc1ccc(CN2CCN(c3ccc(-c4nc(-c5cnn(C)c5)cc5ncc(C#N)n45)cn3)CC2)nc1 ; Cn1cc(-c2cc3ncc(C#N)n3c(-c3ccc(N4CCN(Cc5ccccc5)CC4)nc3)n2)cn1 ; COc1ccc(CN2CCN(c3ccc(-c4cc(-c5cnn(CCN6CCOCC6)c5)cc5ncc(C#N)n45)cn3)CC2)cn1]

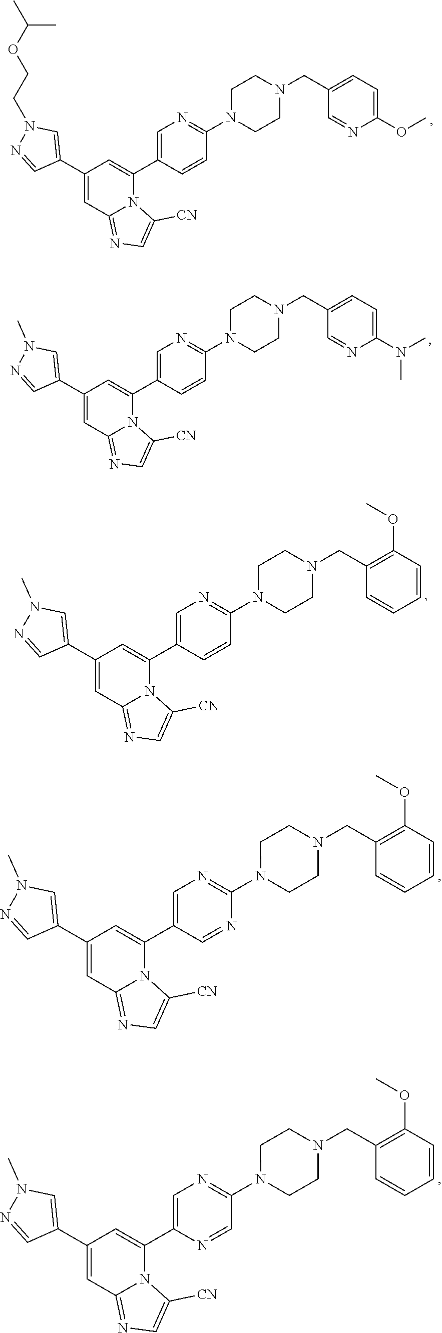 [SMILES: COc1ccc(CN2CCN(c3ccc(-c4cc(-c5cnn(CCOC(C)C)c5)cc5ncc(C#N)n45)cn3)CC2)cn1 ; CN(C)c1ccc(CN2CCN(c3ccc(-c4cc(-c5cnn(C)c5)cc5ncc(C#N)n45)cn3)CC2)cn1 ; COc1ccccc1CN1CCN(c2ccc(-c3cc(-c4cnn(C)c4)cc4ncc(C#N)n34)cn2)CC1 ; COc1ccccc1CN1CCN(c2ncc(-c3cc(-c4cnn(C)c4)cc4ncc(C#N)n34)cn2)CC1 ; COc1ccccc1CN1CCN(c2cnc(-c3cc(-c4cnn(C)c4)cc4ncc(C#N)n34)cn2)CC1]

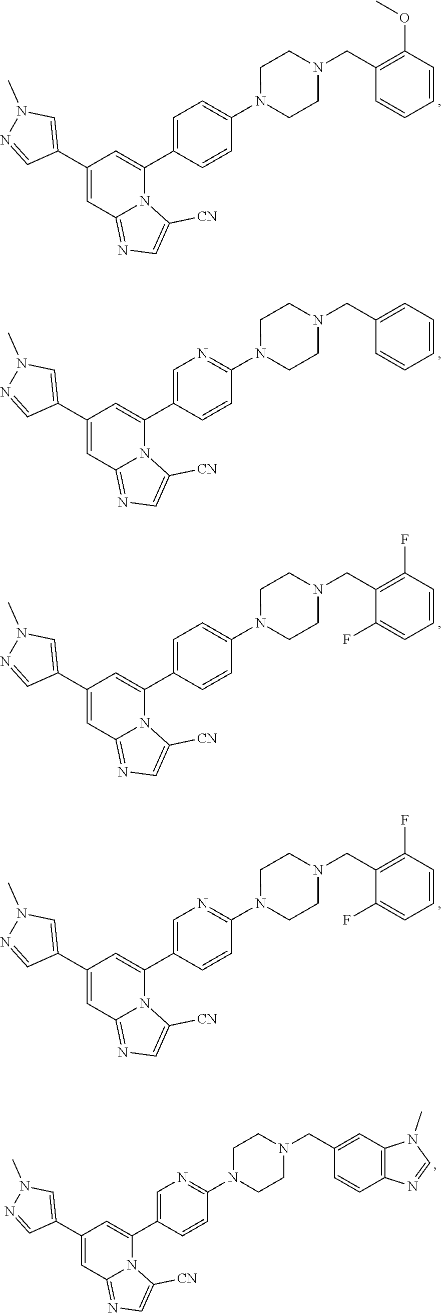 [SMILES: COc1ccccc1CN1CCN(c2ccc(-c3cc(-c4cnn(C)c4)cc4ncc(C#N)n34)cc2)CC1 ; Cn1cc(-c2cc(-c3ccc(N4CCN(Cc5ccccc5)CC4)nc3)n3c(C#N)cnc3c2)cn1 ; Cn1cc(-c2cc(-c3ccc(N4CCN(Cc5c(F)cccc5F)CC4)cc3)n3c(C#N)cnc3c2)cn1 ; Cn1cc(-c2cc(-c3ccc(N4CCN(Cc5c(F)cccc5F)CC4)nc3)n3c(C#N)cnc3c2)cn1 ; Cn1cc(-c2cc(-c3ccc(N4CCN(Cc5ccc6ncn(C)c6c5)CC4)nc3)n3c(C#N)cnc3c2)cn1]

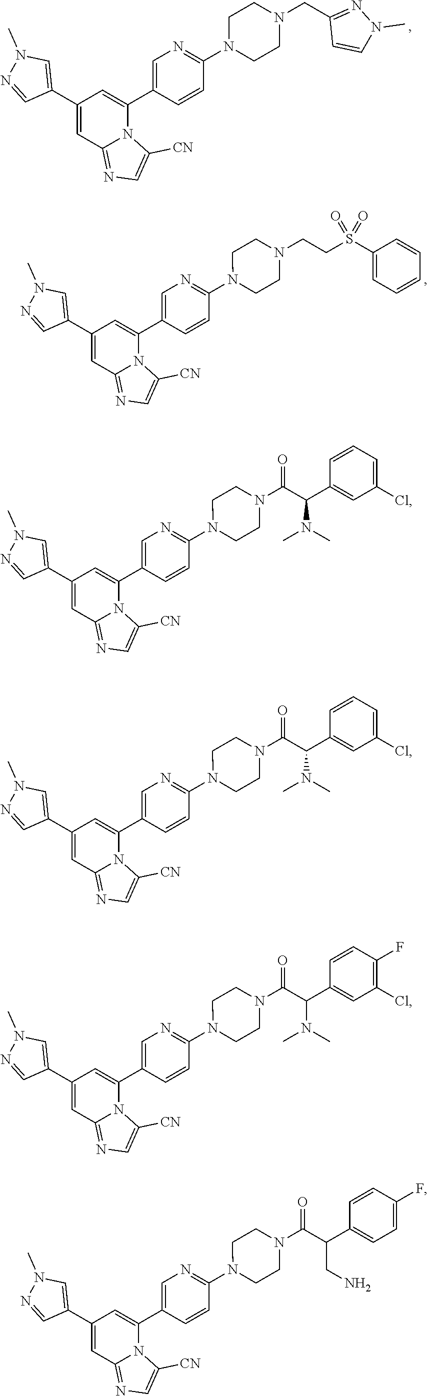 [SMILES: Cn1cc(-c2cc(-c3ccc(N4CCN(Cc5ccn(C)n5)CC4)nc3)n3c(C#N)cnc3c2)cn1 ; Cn1cc(-c2cc(-c3ccc(N4CCN(CCS(=O)(=O)c5ccccc5)CC4)nc3)n3c(C#N)cnc3c2)cn1 ; CN(C)[C@@H](C(=O)N1CCN(c2ccc(-c3cc(-c4cnn(C)c4)cc4ncc(C#N)n34)cn2)CC1)c1cccc(Cl)c1 ; CN(C)[C@H](C(=O)N1CCN(c2ccc(-c3cc(-c4cnn(C)c4)cc4ncc(C#N)n34)cn2)CC1)c1cccc(Cl)c1 ; CN(C)C(C(=O)N1CCN(c2ccc(-c3cc(-c4cnn(C)c4)cc4ncc(C#N)n34)cn2)CC1)c1ccc(F)c(Cl)c1 ; Cn1cc(-c2cc(-c3ccc(N4CCN(C(=O)C(CN)c5ccc(F)cc5)CC4)nc3)n3c(C#N)cnc3c2)cn1]

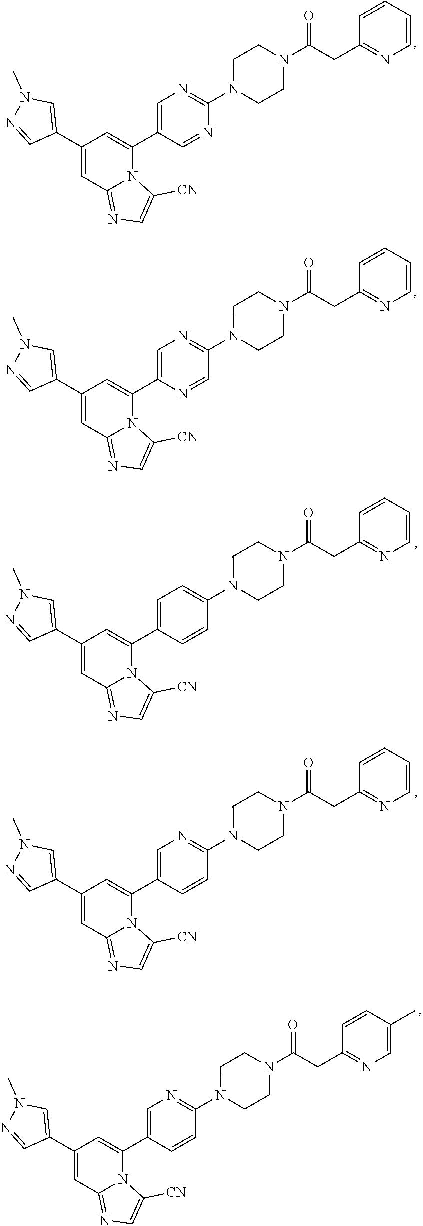 [SMILES: Cn1cc(-c2cc(-c3cnc(N4CCN(C(=O)Cc5ccccn5)CC4)nc3)n3c(C#N)cnc3c2)cn1 ; Cn1cc(-c2cc(-c3cnc(N4CCN(C(=O)Cc5ccccn5)CC4)cn3)n3c(C#N)cnc3c2)cn1 ; Cn1cc(-c2cc(-c3ccc(N4CCN(C(=O)Cc5ccccn5)CC4)cc3)n3c(C#N)cnc3c2)cn1 ; Cn1cc(-c2cc(-c3ccc(N4CCN(C(=O)Cc5ccccn5)CC4)nc3)n3c(C#N)cnc3c2)cn1 ; Cc1ccc(CC(=O)N2CCN(c3ccc(-c4cc(-c5cnn(C)c5)cc5ncc(C#N)n45)cn3)CC2)nc1]

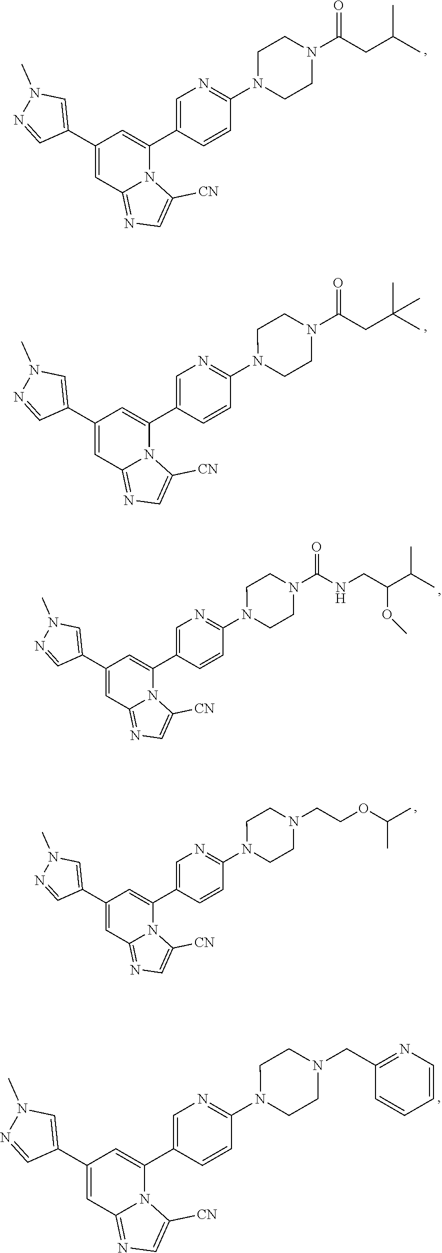 [SMILES: CC(C)CC(=O)N1CCN(c2ccc(-c3cc(-c4cnn(C)c4)cc4ncc(C#N)n34)cn2)CC1 ; Cn1cc(-c2cc(-c3ccc(N4CCN(C(=O)CC(C)(C)C)CC4)nc3)n3c(C#N)cnc3c2)cn1 ; COC(CNC(=O)N1CCN(c2ccc(-c3cc(-c4cnn(C)c4)cc4ncc(C#N)n34)cn2)CC1)C(C)C ; CC(C)OCCN1CCN(c2ccc(-c3cc(-c4cnn(C)c4)cc4ncc(C#N)n34)cn2)CC1 ; Cn1cc(-c2cc(-c3ccc(N4CCN(Cc5ccccn5)CC4)nc3)n3c(C#N)cnc3c2)cn1]

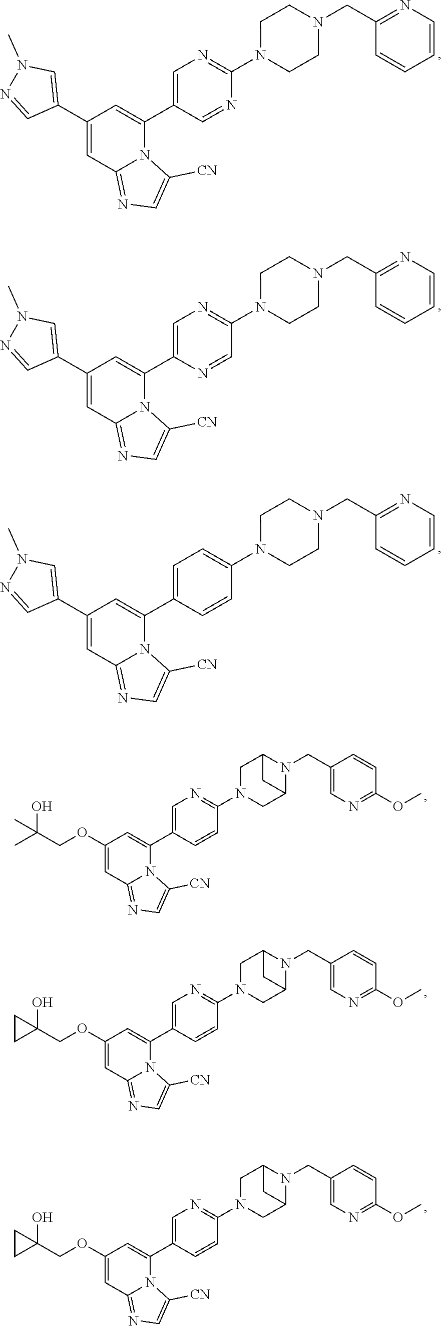 [SMILES: Cn1cc(-c2cc(-c3cnc(N4CCN(Cc5ccccn5)CC4)nc3)n3c(C#N)cnc3c2)cn1 ; Cn1cc(-c2cc(-c3cnc(N4CCN(Cc5ccccn5)CC4)cn3)n3c(C#N)cnc3c2)cn1 ; Cn1cc(-c2cc(-c3ccc(N4CCN(Cc5ccccn5)CC4)cc3)n3c(C#N)cnc3c2)cn1 ; COc1ccc(CN2C3CC2CN(c2ccc(-c4cc(OCC(C)(C)O)cc5ncc(C#N)n45)cn2)C3)cn1 ; COc1ccc(CN2C3CC2CN(c2ccc(-c4cc(OCC5(O)CC5)cc5ncc(C#N)n45)cn2)C3)cn1 ; COc1ccc(CN2C3CC2CN(c2ccc(-c4cc(OCC5(O)CC5)cc5ncc(C#N)n45)cn2)C3)cn1]

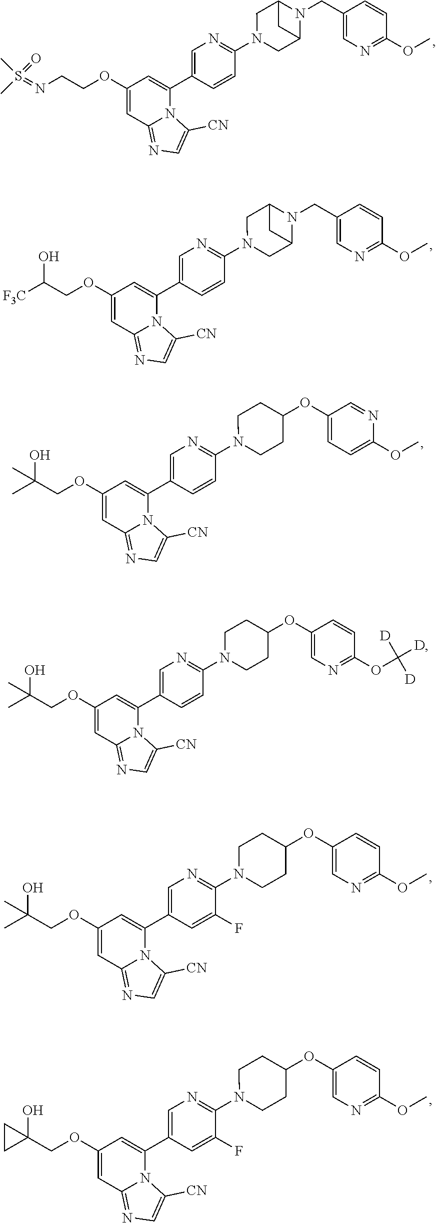

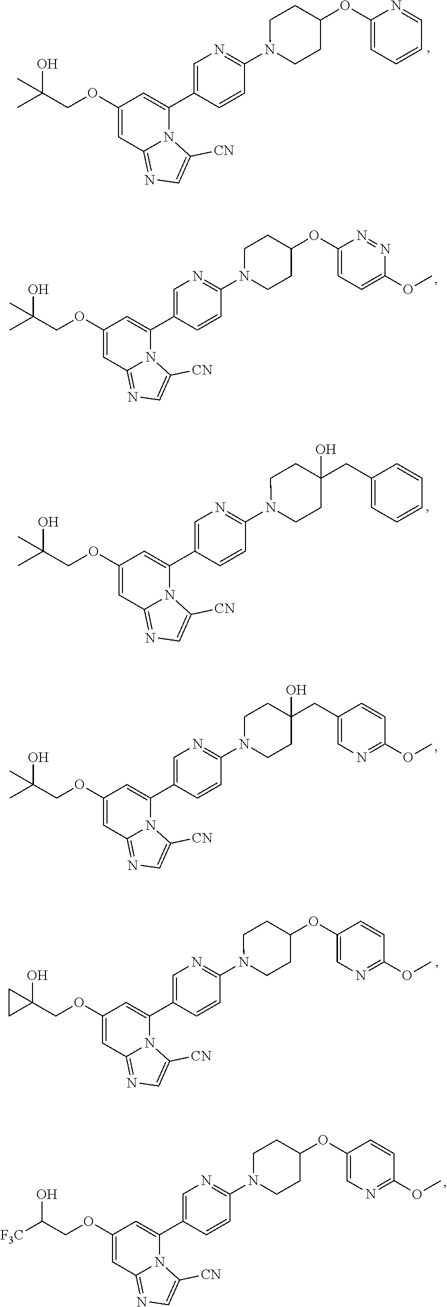

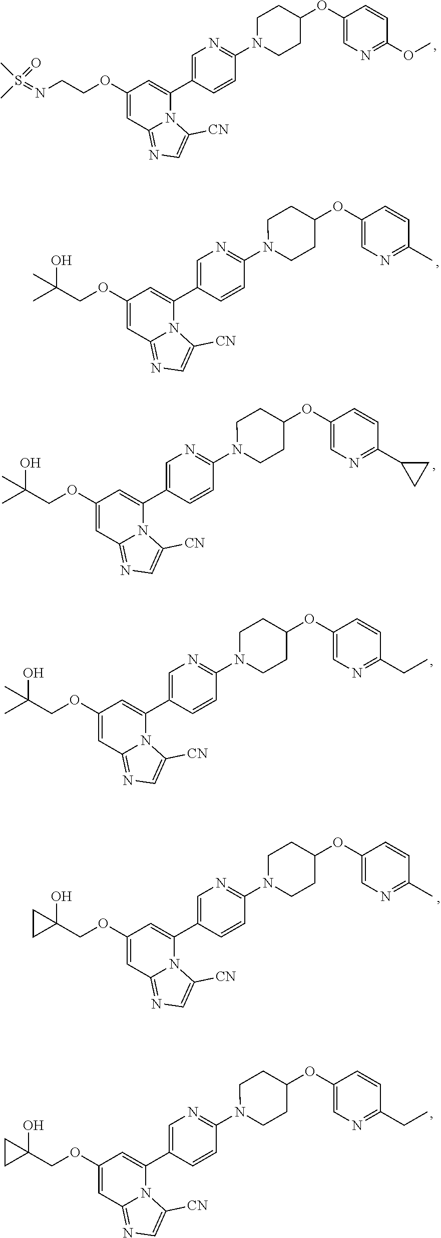

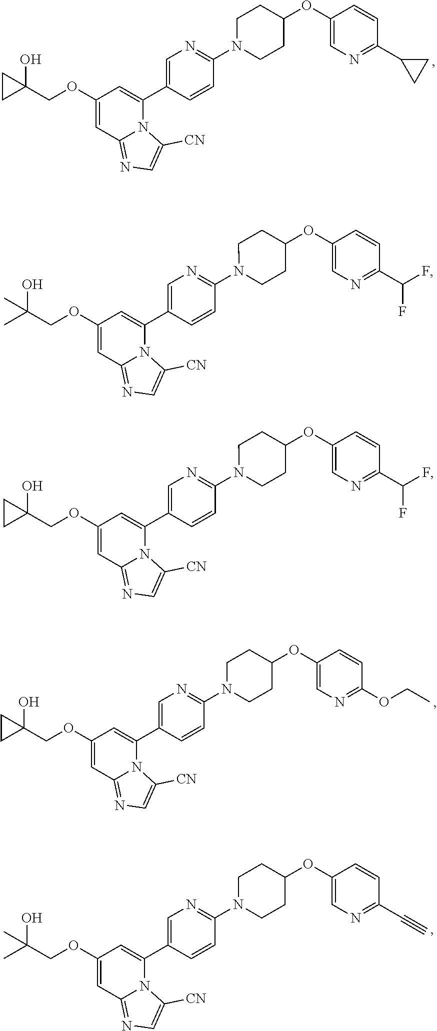

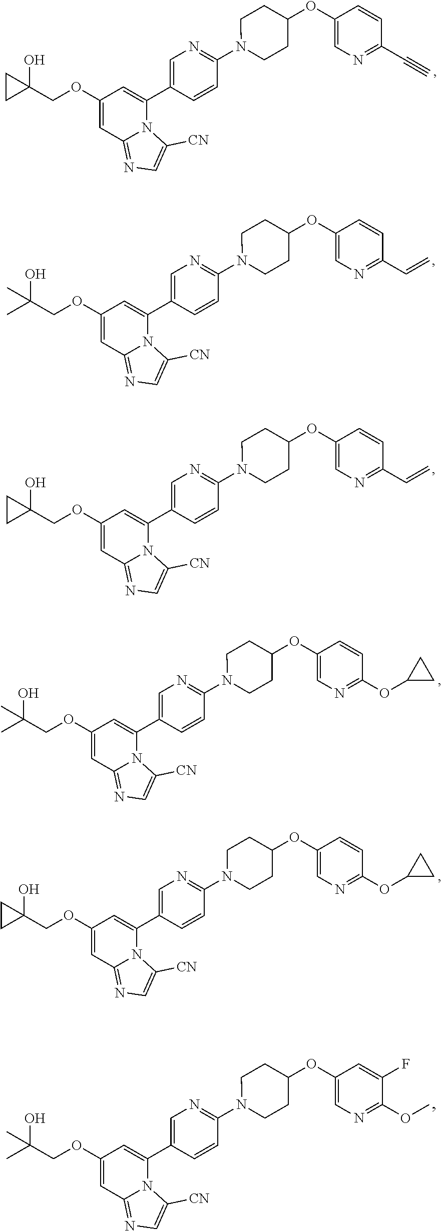

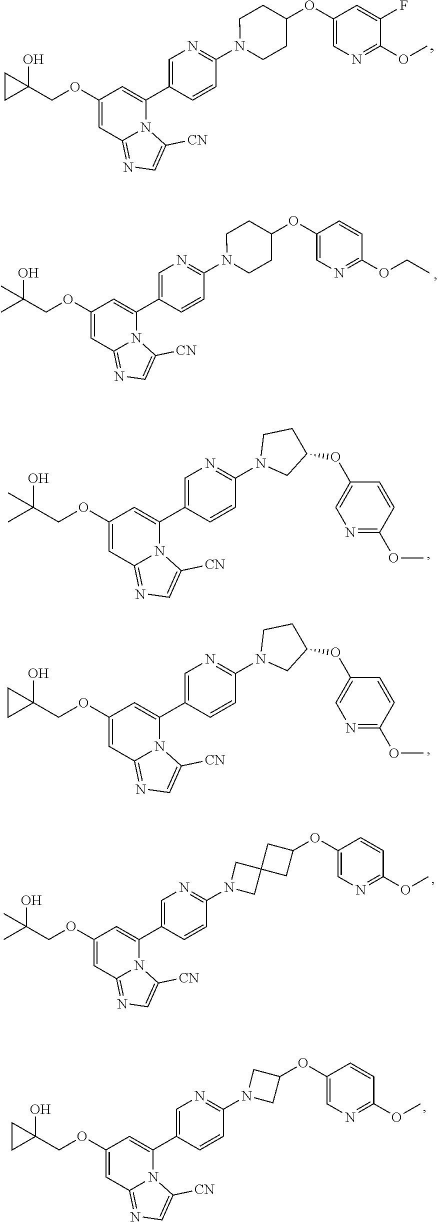

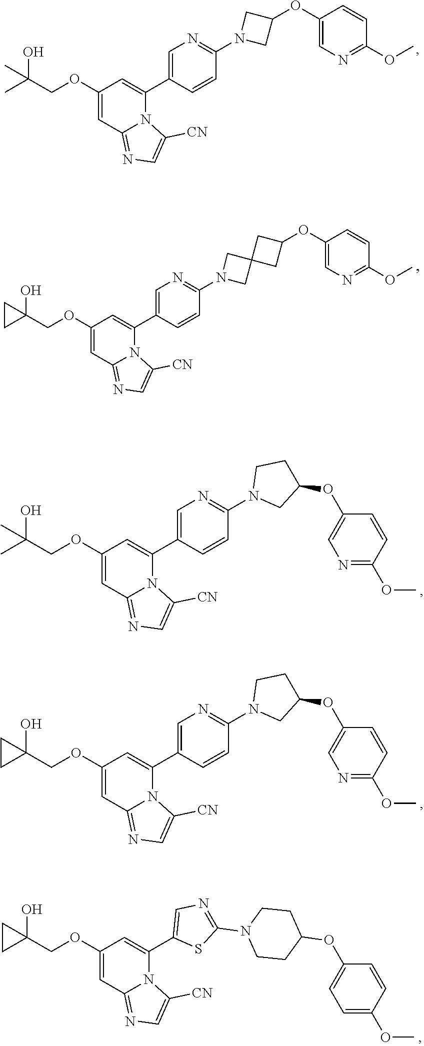

or pharmaceutically acceptable salts thereof.

In another Embodiment (28), the invention provides a pharmaceutical composition comprising a compound of any one of Embodiments (1) to (27) or a pharmaceutically acceptable salt thereof and at least one pharmaceutically acceptable carrier.

In another Embodiment (29), the invention provides a method of treating, ameliorating or preventing a condition, which responds to inhibition of RET, comprising administering to a subject in need of such treatment an effective amount of a compound of any one of Embodiments (1) to (27), or a pharmaceutically acceptable salt thereof, or of at least one pharmaceutical composition thereof, and optionally in combination with a second therapeutic agent.

In another Embodiment (30), the invention provides a use of a compound of any one of Embodiments (1) to (27) or a pharmaceutically acceptable salt thereof in the preparation of a medicament for treating a condition mediated by RET.

In another of its aspects, there is provided a pharmaceutical composition comprising a compound disclosed herein, or a pharmaceutically acceptable salts thereof.

In yet another of its aspects, there is provided a kit comprising a compound disclosed herein, or a pharmaceutically acceptable salts thereof; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound disclosed herein, or a pharmaceutically acceptable salts thereof; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound disclosed herein, or a pharmaceutically acceptable salts thereof.

In another of its aspects, there is provided a method of inhibiting RET kinase comprising contacting the RET kinase with a compound disclosed herein, or a pharmaceutically acceptable salts thereof.

In yet another of its aspects, there is provided a method of inhibiting RET kinase comprising causing a compound disclosed herein, or a pharmaceutically acceptable salts thereof to be present in a subject in order to inhibit RET kinase in vivo.

In a further of its aspects, there is provided a method of inhibiting RET kinase comprising administering a first compound to a subject that is converted in vivo to a second compound wherein the second compound inhibits RET kinase in vivo, the second compound being a compound according to any one of the above embodiments and variations.

In another of its aspects, there is provided a method of treating a disease state for which RET kinase possesses activity that contributes to the pathology and/or symptomology of the disease state, the method comprising causing a compound disclosed herein, or a pharmaceutically acceptable salts thereof to be present in a subject in a therapeutically effective amount for the disease state.

In a further of its aspects, there is provided a method of treating a disease state for which RET kinase possesses activity that contributes to the pathology and/or symptomology of the disease state, the method comprising administering a first compound to a subject that is converted in vivo to a second compound wherein the second compound inhibits RET kinase in vivo. It is noted that the compounds of the present invention may be the first or second compounds.

In one variation of each of the above methods the disease state is selected from the group consisting of cancerous hyperproliferative disorders (e.g., brain, lung, squamous cell, bladder, gastric, pancreatic, breast, head, neck, renal, kidney, ovarian, prostate, colorectal, epidermoid, esophageal, testicular, gynecological or thyroid cancer); non-cancerous hyperproliferative disorders (e.g., benign hyperplasia of the skin (e.g., psoriasis), restenosis, and benign prostatic hypertrophy (BPH)); pancreatitis; kidney disease; pain; preventing blastocyte implantation; treating diseases related to vasculogenesis or angiogenesis (e.g., tumor angiogenesis, acute and chronic inflammatory disease such as rheumatoid arthritis, atherosclerosis, inflammatory bowel disease, skin diseases such as psoriasis, exzema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer); asthma; neutrophil chemotaxis (e.g., reperfusion injury in myocardial infarction and stroke and inflammatory arthritis); septic shock; T-cell mediated diseases where immune suppression would be of value (e.g., the prevention of organ transplant rejection, graft versus host disease, lupus erythematosus, multiple sclerosis, and rheumatoid arthritis); atherosclerosis; inhibition of keratinocyte responses to growth factor cocktails; chronic obstructive pulmonary disease (COPD) and other diseases.

In another of its aspects, there is provided a method of treating a disease state for which a mutation in RET gene contributes to the pathology and/or symptomology of the disease state including, for example, melanomas, lung cancer, colon cancer and other tumor types.

In still another of its aspects, the present invention relates to the use of a compound of any of the above embodiments and variations as a medicament. In yet another of its aspects, the present invention relates to the use of a compound according to any one of the above embodiments and variations in the manufacture of a medicament for inhibiting RET kinase.

In a further of its aspects, the present invention relates to the use of a compound according to any one of the above embodiments and variations in the manufacture of a medicament for treating a disease state for which RET kinase possesses activity that contributes to the pathology and/or symptomology of the disease state.

Administration and Pharmaceutical Compositions

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors known to those of ordinary skill in the art. For example, for the treatment of neoplastic diseases and immune system disorders, the required dosage will also vary depending on the mode of administration, the particular condition to be treated and the effect desired.

In general, satisfactory results are indicated to be obtained systemically at daily dosages of from about 0.001 to about 100 mg/kg per body weight, or particularly, from about 0.03 to 2.5 mg/kg per body weight. An indicated daily dosage in the larger mammal, e.g. humans, may be in the range from about 0.5 mg to about 2000 mg, or more particularly, from about 0.5 mg to about 1000 mg, conveniently administered, for example, in divided doses up to four times a day or in retard form. Suitable unit dosage forms for oral administration comprise from ca. 1 to 50 mg active ingredient.

Compounds of the disclosure may be administered as pharmaceutical compositions by any conventional route; for example, enterally, e.g., orally, e.g., in the form of tablets or capsules; parenterally, e.g., in the form of injectable solutions or suspensions; or topically, e.g., in the form of lotions, gels, ointments or creams, or in a nasal or suppository form.

Pharmaceutical compositions comprising a compound of the present disclosure in free form or in a pharmaceutically acceptable salt form in association with at least one pharmaceutically acceptable carrier or diluent may be manufactured in a conventional manner by mixing, granulating, coating, dissolving or lyophilizing processes. For example, pharmaceutical compositions comprising a compound of the disclosure in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

In one embodiment, the pharmaceutical compositions are solutions of the active ingredient, including suspensions or dispersions, such as isotonic aqueous solutions. In the case of lyophilized compositions comprising the active ingredient alone or together with a carrier such as mannitol, dispersions or suspensions can be made up before use. The pharmaceutical compositions may be sterilized and/or contain adjuvants, such as preserving, stabilizing, wetting or emulsifying agents, solution promoters, salts for regulating the osmotic pressure and/or buffers. Suitable preservatives include but are not limited to antioxidants such as ascorbic acid, or microbicides, such as sorbic acid or benzoic acid. The solutions or suspensions may further comprise viscosity-increasing agents, including but not limited to, sodium carboxymethylcellulose, carboxymethylcellulose, dextran, polyvinylpyrrolidone, gelatins, or solubilizers, e.g. Tween 80 (polyoxyethylene(20)sorbitan mono-oleate).

Suspensions in oil may comprise as the oil component the vegetable, synthetic, or semi-synthetic oils customary for injection purposes. Examples include but are not limited to liquid fatty acid esters that contain as the acid component a long-chained fatty acid having 8-22 carbon atoms, or in some embodiments, 12-22 carbon atoms. Suitable liquid fatty acid esters include but are not limited to lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid and linoleic acid, and if desired, may contain antioxidants, for example vitamin E, 3-carotene or 3,5-di-tert-butyl-hydroxytoluene. The alcohol component of these fatty acid esters may have six carbon atoms and may be monovalent or polyvalent, for example a mono-, di- or trivalent, alcohol. Suitable alcohol components include but are not limited to methanol, ethanol, propanol, butanol or pentanol or isomers thereof; glycol and glycerol.

Other suitable fatty acid esters include but are not limited ethyl-oleate, isopropyl myristate, isopropyl palmitate, LABRAFIL® M 2375, (polyoxyethylene glycerol), LABRAFIL® M 1944 CS (unsaturated polyglycolized glycerides prepared by alcoholysis of apricot kernel oil and comprising glycerides and polyethylene glycol ester), LABRASOL™ (saturated polyglycolized glycerides prepared by alcoholysis of TCM and comprising glycerides and polyethylene glycol ester; all available from GaKefosse, France), and/or MIGLYOL® 812 (triglyceride of saturated fatty acids of chain length C8 to C12 from Huls AG, Germany), and vegetable oils such as cottonseed oil, almond oil, olive oil, castor oil, sesame oil, soybean oil, or groundnut oil.

Pharmaceutical compositions for oral administration may be obtained, for example, by combining the active ingredient with one or more solid carriers, and if desired, granulating a resulting mixture, and processing the mixture or granules by the inclusion of additional excipients, to form tablets or tablet cores.

Suitable carriers include but are not limited to fillers, such as sugars, for example lactose, saccharose, mannitol or sorbitol, cellulose preparations and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, carboxymethyl starch, crosslinked polyvinylpyrrolidone, alginic acid or a salt thereof, such as sodium alginate. Additional excipients include but are not limited to flow conditioners and lubricants, for example silicic acid, talc, stearic acid or salts thereof, such as magnesium or calcium stearate, and/or polyethylene glycol, or derivatives thereof.

Tablet cores may be provided with suitable, optionally enteric, coatings through the use of, inter alia, concentrated sugar solutions which may comprise gum arable, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different doses of active ingredient.

Pharmaceutical compositions for oral administration may also include hard capsules comprising gelatin or soft-sealed capsules comprising gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient may be dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories comprising a combination of the active ingredient and a suppository base. Suitable suppository bases are, for example, natural or synthetic triglycerides, paraffin hydrocarbons, polyethylene glycols or higher alkanols.

Pharmaceutical compositions suitable for parenteral administration may comprise aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers. The active ingredient, optionally together with excipients, can also be in the form of a lyophilizate and can be made into a solution before parenteral administration by the addition of suitable solvents. Solutions such as are used, for example, for parenteral administration can also be employed as infusion solutions. The manufacture of injectable preparations is usually carried out under sterile conditions, as is the filling, for example, into ampoules or vials, and the sealing of the containers.

The disclosure also provides for a pharmaceutical combination, e.g. a kit, comprising a) a first agent which is a compound of the disclosure as disclosed herein, in free form or in pharmaceutically acceptable salt form, and b) at least one co-agent. The kit can comprise instructions for its administration.

Combination Therapies

The compounds or pharmaceutical acceptable salts of the disclosure may be administered as the sole therapy, or together with other therapeutic agent or agents.

For example, the therapeutic effectiveness of one of the compounds described herein may be enhanced by administration of an adjuvant (i.e. by itself the adjuvant may only have minimal therapeutic benefit, but in combination with another therapeutic agent, the overall therapeutic benefit to the individual is enhanced). Or, by way of example only, the benefit experienced by an individual may be increased by administering one of the compounds described herein with another therapeutic agent that also has therapeutic benefit. By way of example only, in a treatment for gout involving administration of one of the compounds described herein, increased therapeutic benefit may result by also providing the individual with another therapeutic agent for gout. Or, by way of example only, if one of the side effects experienced by an individual upon receiving one of the compounds described herein is nausea, then it may be appropriate to administer an anti-nausea agent in combination with the compound. Or, the additional therapy or therapies include, but are not limited to physiotherapy, psychotherapy, radiation therapy, application of compresses to a diseased area, rest, altered diet, and the like. Regardless of the disease, disorder or condition being treated, the overall benefit experienced by the individual may be additive of the two therapies or the individual may experience a synergistic benefit.

In the instances where the compounds described herein are administered in combination with other therapeutic agents, the compounds described herein may be administered in the same pharmaceutical composition as other therapeutic agents, or because of different physical and chemical characteristics, be administered by a different route. For example, the compounds described herein may be administered orally to generate and maintain good blood levels thereof, while the other therapeutic agent may be administered intravenously. Thus the compounds described herein may be administered concurrently, sequentially or dosed separately to other therapeutic agents.

### EXAMPLES

Various methods may be developed for synthesizing a compound of formula (I) or a pharmaceutically acceptable salt thereof. Representative methods for synthesizing a compound of formula (I) or a pharmaceutically acceptable salt thereof are provided in the Examples. It is noted, however, that a compound of formula (I) or a pharmaceutically acceptable salt thereof may also be synthesized by other synthetic routes that others may devise.

It will be readily recognized that certain compounds of formula (I) have atoms with linkages to other atoms that confer a particular stereochemistry to the compound (e.g., chiral centers). It is recognized that synthesis of a compound of formula (I) or a pharmaceutically acceptable salt thereof may result in the creation of mixtures of different stereoisomers (enantiomers, diastereomers). Unless a particular stereochemistry is specified, recitation of a compound is intended to encompass all of the different possible stereoisomers.

Ae compound of formula (I) can also be prepared as a pharmaceutically acceptable acid addition salt by, for example, reacting the free base form of the at least one compound with a pharmaceutically acceptable inorganic or organic acid. Alternatively, a pharmaceutically acceptable base addition salt of the at least one compound of formula (I) can be prepared by, for example, reacting the free acid form of the at least one compound with a pharmaceutically acceptable inorganic or organic base. Inorganic and organic acids and bases suitable for the preparation of the pharmaceutically acceptable salts of compounds of formula (I) are set forth in the definitions section of this Application. Alternatively, the salt forms of the compounds of formula (I) can be prepared using salts of the starting materials or intermediates.

The free acid or free base forms of the compounds of formula (I) can be prepared from the corresponding base addition salt or acid addition salt form. For example, a compound of formula (I) in an acid addition salt form can be converted to the corresponding free base thereof by treating with a suitable base (e.g., ammonium hydroxide solution, sodium hydroxide, and the like). A compound of formula (I) in a base addition salt form can be converted to the corresponding free acid thereof by, for example, treating with a suitable acid (e.g., hydrochloric acid, etc).

The N-oxides of the a compound of formula (I) or a pharmaceutically acceptable salt thereof can be prepared by methods known to those of ordinary skill in the art. For example, N-oxides can be prepared by treating an unoxidized form of the compound of formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0 to 80° C. Alternatively, the N-oxides of the compounds of formula (I) can be prepared from the N-oxide of an appropriate starting material.

Compounds of formula (I) in an unoxidized form can be prepared from N-oxides of compounds of formula (I) by, for example, treating with a reducing agent (e.g., sulfur, sulfur dioxide, triphenyl phosphine, lithium borohydride, sodium borohydride, phosphorus trichloride, tribromide, and the like) in an suitable inert organic solvent (e.g., acetonitrile, ethanol, aqueous dioxane, and the like) at 0 to 80° C.

Protected derivatives of the compounds of formula (I) can be made by methods known to those of ordinary skill in the art. A detailed description of the techniques applicable to the creation of protecting groups and their removal can be found in T. W. Greene, Protecting Groups in Organic Synthesis, 3rd edition, John Wiley & Sons, Inc. 1999.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. For example, the following abbreviations may be used in the examples and throughout the specification: g (grams); mg (milligrams); L (liters); mL (milliliters); μL (microliters); psi (pounds per square inch); M (molar); mM (millimolar); i.v. (intravenous); Hz (Hertz); MHz (megahertz); mol (moles); mmol (millimoles); RT (room temperature); min (minutes); h (hours); mp (melting point); TLC (thin layer chromatography); Rt (retention time); RP (reverse phase); MeOH (methanol); i-PrOH (isopropanol); TEA (triethylamine); TFA (trifluoroacetic acid); TFAA (trifluoroacetic anhydride); THF (tetrahydrofuran); DMSO (dimethyl sulfoxide); EtOAc (ethyl acetate); DME (1,2-dimethoxyethane); DCM (dichloromethane); DCE (dichloroethane); DMF (N,N-dimethylformamide); DMPU (N,N′-dimethylpropyleneurea); CDI (1,1-carbonyldiimidazole); IBCF (isobutyl chloroformate); HOAc (acetic acid); HOSu (N-hydroxysuccinimide); HOBT (1-hydroxybenzotriazole); Et₂O (diethyl ether); EDCI (1-(3-dimethylaminopropyl)-3-ethylcarbodiimide hydrochloride); BOC (tert-butyloxycarbonyl); FMOC (9-fluorenylmethoxycarbonyl); DCC (dicyclohexylcarbodiimide); CBZ (benzyloxycarbonyl); Ac (acetyl); atm (atmosphere); TMSE (2-(trimethylsilyl)ethyl); TMS (trimethylsilyl); TIPS (triisopropylsilyl); TBS (t-butyldimethylsilyl); DMAP (4-dimethylaminopyridine); Me (methyl); OMe (methoxy); Et (ethyl); tBu (tert-butyl); HPLC (high pressure liquid chomatography); BOP (bis(2-oxo-3-oxazolidinyl)phosphinic chloride); TBAF (tetra-n-butylammonium fluoride); m-CPBA (meta-chloroperbenzoic acid).

References to ether or Et₂O are to diethyl ether; brine refers to a saturated aqueous solution of NaCl. Unless otherwise indicated, all temperatures are expressed in ° C. (degrees Centigrade). All reactions were conducted under an inert atmosphere at RT unless otherwise noted.

¹H NMR spectra were recorded on a Varian Mercury Plus 400. Chemical shifts are expressed in parts per million (ppm). Coupling constants are in units of hertz (Hz). Splitting patterns describe apparent multiplicities and are designated as s (singlet), d (doublet), t (triplet), q (quartet), m (multiplet) and br (broad).

Low-resolution mass spectra (MS) and compound purity data were acquired on a Shimadzu LC/MS single quadrapole system equipped with electrospray ionization (ESI) source, UV detector (220 and 254 nm), and evaporative light scattering detector (ELSD). Thin-layer chromatography was performed on 0.25 mm Superchemgroup silica gel plates (60F-254), visualized with UV light, 5% ethanolic phosphomolybdic acid, ninhydrin, or p-anisaldehyde solution. Flash column chromatography was performed on silica gel (200-300 mesh, Branch of Qingdao Haiyang Chemical Co., Ltd).

Synthetic Schemes

Synthetic methods for preparing the compounds of the present invention are illustrated in the following Schemes and Examples. Starting materials are commercially available or may be made according to procedures known in the art or as illustrated herein.

In the reactions described herein after it may be necessary to protect reactive functional groups, for example hydroxyl, amino, imino, thio or carboxyl groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991

Synthetic methods for preparing the compounds of the present invention are illustrated in the following Schemes and Examples. Starting materials are commercially available or may be made according to procedures known in the art or as illustrated herein.

The intermediates shown in the following schemes are either known in the literature or may be prepared by a variety of methods familiar to those skilled in the art.

One synthetic approach of compounds of formula I of the present disclosure is shown in Scheme 1. Starting from the intermediates II, which is either commercially available or known in the literature, intermediates of formula IV can be prepared by the coupling of II with the intermediates III using transitional metal catalyzed cross coupling reactions known in the literature. Further manipulation of functional group Z₂ in intermediates IV through reactions such as cross coupling or derivatization reactions leads to compounds of formula I.

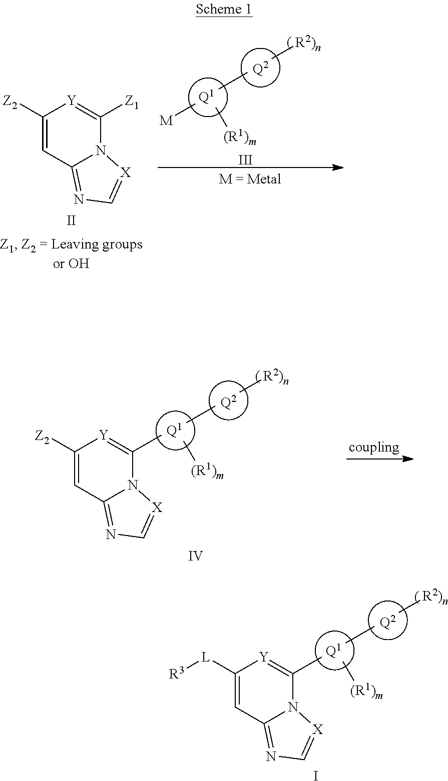

As an illustration of the synthesis of compounds of formula I, one of the synthetic approach of the compounds of formula Ta is outlined in Scheme 2. Starting from the commercially available symmetrical dibromide IIa-A, amine IIa-D can be obtained by selective Buchwald amination and Boc deprotection. The bicyclic heterocyle IIa can be readily prepared from amine IIa-D through a sequence of transformations including condensation with DMF-DMA IIa-E, alkylation of the pyridine ring with bromoaectonitrile IIa-G and intramolecular cyclization effected by an organic base such as DIPEA. Coupling of halide IIa with boronic acid IIIa using transitional metal catalysed coupling conditions such as Suzuki reaction provides the tricyclic intermediate IVa. Conversion of the methoxy group of IVa into a phenolic hydroxyl group promoted by a Lewis acid such as AlCl₃ in a solvent such as DCE and reaction of the resulting phenolic hydroxyl group with the epoxide Ia-B leads to compound of formula of Ia-C. Depending on the functionalities in Ia, the pyridine part of Ia-C can be further modified through reactions such as S_{N}Ar substitutions accordingly.

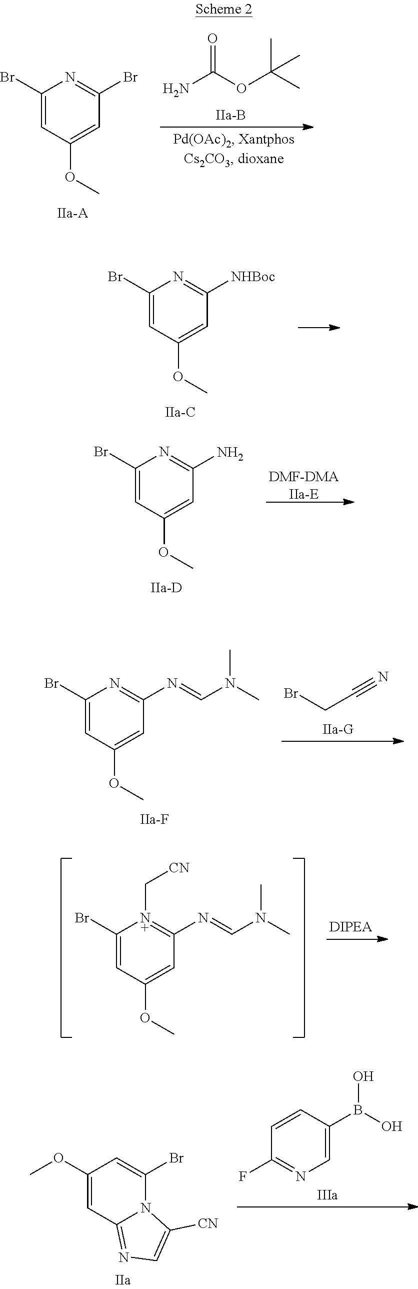

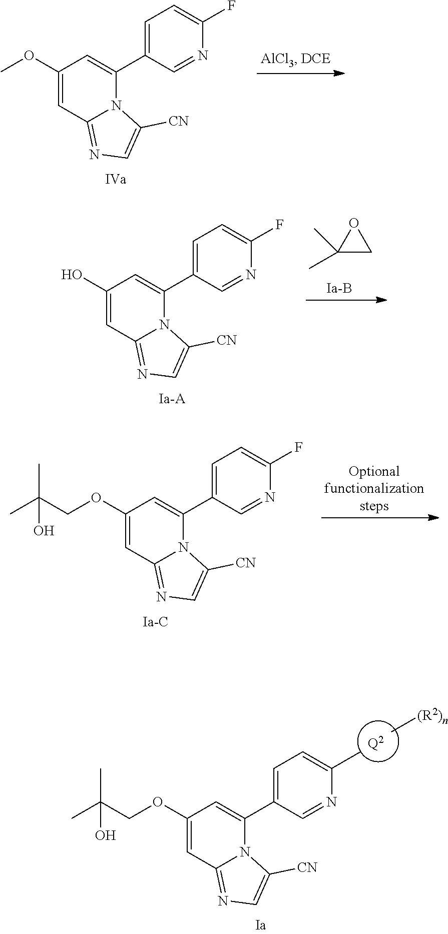

As an further illustration of the synthesis of compounds of formula I, one of the synthetic approach of the compounds of formula Ib is outlined in Scheme 3. Conversation of the hydroxyl group in Ia-A into a leaving group such as OTf leads to Ib-A. The Suzuki cross coupling between halide Ib-A and boronic acid Vb gives Ib-B. Further functionalization of the pyridine part of Ib-B can be effected through reactions such as S_{N}Ar substitutions followed by other necessary derivatization reactions to give compounds of formula Ib.

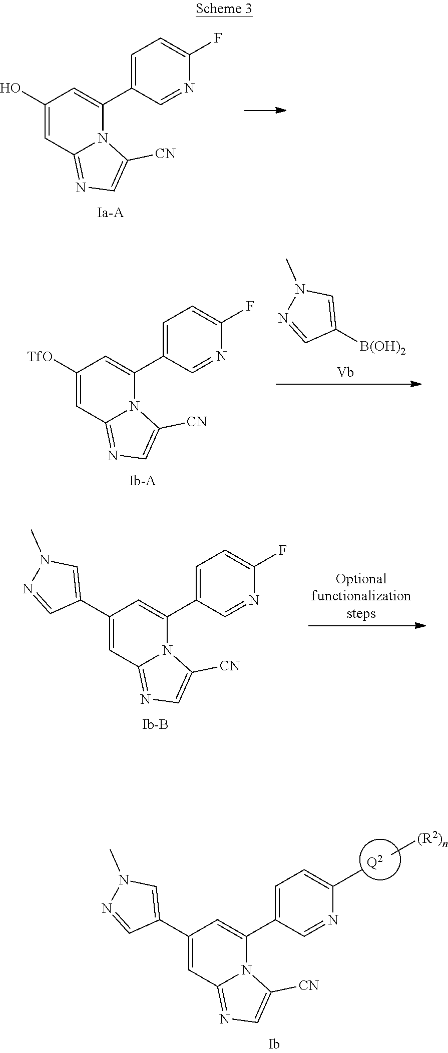

In some cases, the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Example 1

### 5-(6-(4-(4-Fluorobenzyl)piperazin-1-yl)pyridin-3-yl)-7-(1-methyl-1H-pyrazol-4-yl)imidazo[1,2-a]pyridine-3-carbonitrile (1)

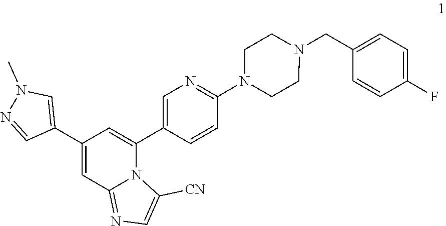 [SMILES: Cn1cc(-c2cc(-c3ccc(N4CCN(Cc5ccc(F)cc5)CC4)nc3)n3c(C#N)cnc3c2)cn1]

### Tert-butyl (6-bromo-4-methoxypyridin-2-yl)carbamate (1a)

Tert-butyl (6-bromo-4-methoxypyridin-2-yl)carbamate (1a) was prepared according to the method described in WO2017/205536.

### 6-Bromo-4-methoxypyridin-2-amine (1b)

The mixture of tert-butyl (6-bromo-4-methoxypyridin-2-yl)carbamate (1a) (1.30 g, 4.29 mmol) in DCM (5.0 mL) and TFA (5.0 mL) was stirred at RT for 0.5 h. The mixture was concentrated and diluted with H₂O (20 mL), and the mixture was neutralized with NaHCO₃, extracted with EA (2×50 mL), washed with brine, dried over Na₂SO₄, filtered and concentrated to give the title compound 6-bromo-4-methoxypyridin-2-amine (1b). MS-ESI (m/z): 203/205 [M+1]⁺.

### 5-Bromo-7-methoxyimidazo[1,2-a]pyridine-3-carbonitrile (1c)

The mixture of 6-bromo-4-methoxypyridin-2-amine (1b) (200 mg, 1.00 mmol) and DMF-DMA was stirred at 100° C. for 1 h. The mixture was concentrated and dissolved in AcCN (3.0 mL), 2-bromoacetonitrile (480 mg, 4.00 mmol) was added and stirred at 80° C. for 6 h. Then the mixture was cooled to RT and DIPEA (645 mg, 5.00 mmol) was added and stirred for 2 h. The precipitate was collected by filtration and dried to give the title compound 5-bromo-7-methoxyimidazo[1,2-a]pyridine-3-carbonitrile (1c). MS-ESI (m/z): 252/254 [M+1]+.

### 5-(6-Fluoropyridin-3-yl)-7-methoxyimidazo[1,2-a]pyridine-3-carbonitrile (1d)

A mixture of 5-bromo-7-methoxyimidazo[1,2-a]pyrimidine-3-carbonitrile (1c) (70 mg, 0.28 mmol), (4-fluorophenyl)boronic acid (47 mg, 0.33 mmol) and Pd(PPh₃)₄ (16 mg, 0.014 mmol) in dioxane (3 mL) and 2 M Na₂CO₃ aqueous solution (0.42 ml) was stirred at 80° C. under N₂ atmosphere for 6 h. The mixture was concentrated and purified by column chromatography on silica gel, eluting with DCM/MeOH (30:1) to give the title compound 5-(6-fluoropyridin-3-yl)-7-methoxyimidazo[1,2-a]pyridine-3-carbonitrile (1d). MS-ESI (m/z): 269 [M+1]+.

### 5-(6-Fluoropyridin-3-yl)-7-hydroxyimidazo[1,2-a]pyridine-3-carbonitrile (1e)

The mixture of 5-(6-fluoropyridin-3-yl)-7-methoxyimidazo[1,2-a]pyridine-3-carbonitrile (1d) (270 mg, 1.00 mmol) and AlCl₃ (540 mg, 4.00 mmol) in DCE (10 mL) was stirred at 80° C. for 6 h. The mixture was quenched with Na₂SO₄.10H₂O and stirred at r.t. for 1 h. Then the mixture was filtered and concentrated to give the title compound 5-(6-fluoropyridin-3-yl)-7-hydroxyimidazo[1,2-a]pyridine-3-carbonitrile (1e). MS-ESI (m/z): 255 [M+1]+.

### 3-Cyano-5-(6-fluoropyridin-3-yl)imidazo[1,2-a]pyridin-7-yl trifluoromethanesulfonate (1f)

To a solution of 5-(6-fluoropyridin-3-yl)-7-hydroxyimidazo[1,2-a]pyridine-3-carbonitrile (1e) (254 mg, 1.00 mmol) in DMA (10.0 mL) was added DIPEA (258 mg, 2.00 mmol) and N,N-Bis(trifluoromethylsulfonyl)aniline (393 mg, 1.10 mmol). The reaction was stirred at r.t. for 0.5 h. The mixture was quenched with H₂O, extracted with EtOAc, washed with brine, dried over Na₂SO₄, filtered and concentrated to give a residue. The residue was purified by column chromatography on silica gel, eluting with petroleum ether/EtOAc (10:1˜5:1˜2:1) to give the title compound 3-cyano-5-(6-fluoropyridin-3-yl)imidazo[1,2-a]pyridin-7-yl trifluoromethanesulfonate (1f). MS-ESI (m/z): 387 [M+1]⁺.

### 5-(6-Fluoropyridin-3-yl)-7-(1-methyl-1H-pyrazol-4-yl)imidazo[1,2-a]pyridine-3-carbonitrile (1g)

A mixture of 3-cyano-5-(6-fluoropyridin-3-yl)imidazo[1,2-a]pyridin-7-yl trifluoromethanesulfonate (1f) (200 mg, 0.520 mmol), 1-methyl-4-(4,4,5,5-tetramethyl-1,3,2-dioxaborolan-2-yl)-1H-pyrazole (162 mg, 0.780 mmol) and Pd(PPh₃)₄ (60.0 mg, 0.0520 mmol) in dioxane (10 mL) and 2 M Na₂CO₃ aqueous solution (0.78 ml) was stirred at 80° C. under N2 atmosphere for 2 h. The mixture was concentrated and purified by column chromatography on silica gel, eluting with DCM/MeOH (30:1-20:1) to give the title compound 5-(6-fluoropyridin-3-yl)-7-(1-methyl-1H-pyrazol-4-yl)imidazo[1,2-a]pyridine-3-carbonitrile (1g). MS-ESI (m/z): 319 [M+1]⁺.

### Tert-butyl 4-(5-(3-cyano-7-(1-methyl-1H-pyrazol-4-yl)imidazo[1,2-a]pyridin-5-yl)pyridin-2-yl)piperazine-1-carboxylate (1h)

A mixture of 5-(6-fluoropyridin-3-yl)-7-(1-methyl-1H-pyrazol-4-yl)imidazo-[1,2-a]pyridine-3-carbonitrile (1g) (100 mg, 0.314 mmol), tert-butyl piperazine-1-carboxylate (117 mg, 0.628 mmol), and K₂CO₃ (217 mg, 1.57 mmol) in DMSO (4 mL) was stirred at 120° C. under N2 atmosphere for 15 h. The mixture was cooled to RT and diluted with H₂O, extracted with DCM (3×50 mL). The organic phase was washed with H₂O, dried over Na₂SO₄ and concentrated. The residue was purified by column chromatography on silica gel, eluting with DCM/MeOH (100:1-40:1) to give the title compound tert-butyl 4-(5-(3-cyano-7-(1-methyl-1H-pyrazol-4-yl)imidazo[1,2-a]pyridin-5-yl)pyridin-2-yl)piperazine-1-carboxylate (1h). MS-ESI (m/z): 485 [M+1]+.

### 7-(1-Methyl-1H-pyrazol-4-yl)-5-(6-(piperazin-1-yl)pyridin-3-yl)imidazo[1,2-a]pyridine-3-carbonitrile (1i)

To a solution of tert-butyl 4-(5-(3-cyano-7-(1-methyl-1H-pyrazol-4-yl)-imidazo[1,2-a]pyridin-5-yl)pyridin-2-yl)piperazine-1-carboxylate (1h) (110 mg, 0.227 mmol) in DCM (4 mL) was added TFA (1 ml). The mixture was stirred at RT for 1 h. The mixture was concentrated. The residue was diluted with sat. NaHCO₃ aqueous solution (20 mL) and extracted with DCM/MeOH (10:1) (4×20 ml). The organic phase was washed with H₂O, dried over Na₂SO₄ and concentrated. The residue was purified by column chromatography on silica gel, eluting with DCM/MeOH (50:1-10:1) to give the title compound 7-(1-methyl-1H-pyrazol-4-yl)-5-(6-(piperazin-1-yl)pyridin-3-yl)imidazo[1,2-a]pyridine-3-carbonitrile (li). MS-ESI (m/z): 385 [M+1]+.

### 5-(6-(4-(4-Fluorobenzyl)piperazin-1-yl)pyridin-3-yl)-7-(1-methyl-1H-pyrazol-4-yl)imidazo[1,2-a]pyridine-3-carbonitrile (1)

To a solution of 7-(1-methyl-1H-pyrazol-4-yl)-5-(6-(piperazin-1-yl)pyridin-3-yl)imidazo[1,2-a]pyridine-3-carbonitrile (li) (10 mg, 0.026 mmol) in DCM (1 mL) was added 4-fluorobenzaldehyde (6.4 mg, 0.052 mmol), followed by NaBH(OAc)₃ (17 mg, 0.078 mmol). Then the mixture was stirred at RT for 0.5 h. The mixture was poured into sat. NaHCO₃ aqueous solution and extracted with DCM (4×20 ml). The organic phase was washed with H₂O, dried over Na₂SO₄ and concentrated. The residue was purified by column chromatography on silica gel, eluting with DCM/MeOH (20:1) to give the title compound 5-(6-(4-(4-fluorobenzyl)piperazin-1-yl)pyridin-3-yl)-7-(1-methyl-1H-pyrazol-4-yl)imidazo[1,2-a]pyridine-3-carbonitrile (1). MS-ESI (m/z): 493 [M+1]⁺.

### Example 2

### 5-(6-(4-(6-Methoxynicotinoyl)piperazin-1-yl)pyridin-3-yl)-7-(1-methyl-1H-pyrazol-4-yl)imidazo[1,2-a]pyridine-3-carbonitrile (2)

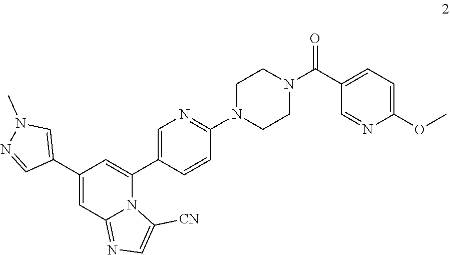 [SMILES: COc1ccc(C(=O)N2CCN(c3ccc(-c4cc(-c5cnn(C)c5)cc5ncc(C#N)n45)cn3)CC2)cn1]

To a solution of 7-(1-methyl-1H-pyrazol-4-yl)-5-(6-(piperazin-1-yl)pyridin-3-yl)imidazo[1,2-a]pyridine-3-carbonitrile (li) (10 mg, 0.026 mmol) in DCM (1.0 mL) was added 6-methoxynicotinic acid (6.0 mg, 0.039 mmol), EDCI (15 mg, 0.078 mmol), HOBT (11 mg, 0.078 mmol), followed by TEA (8.0 mg, 0.083 mmol). After being stirred at RT for overnight, the mixture was concentrated and the residue was purified by column chromatography on silica gel, eluting with DCM/MeOH (30:1-10:1) to give the title compound 5-(6-(4-(6-methoxynicotinoyl)piperazin-1-yl)pyridin-3-yl)-7-(1-methyl-1H-pyrazol-4-yl)imidazo[1,2-a]pyridine-3-carbonitrile (2). MS-ESI (m/z): 520 [M+1]+.

Following essentially the same procedures described for Examples 1-2, Examples 3-55 listed in Table 1 were prepared from the appropriate starting materials which are either commercially available or known in the literature. The structures and names of Examples 3-55 are given in Table 1.

TABLE 1

| EXAMPLE | STRUCTURE | NAME | DATA |
|---|---|---|---|
| 3 | 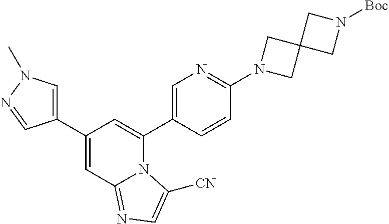 [SMILES: Cn1cc(-c2cc(-c3ccc(N4CC5(CN(C(=O)OC(C)(C)C)C5)C4)nc3)n3c(C#N)cnc3c2)cn1] | tert-butyl 6-(5-(3-cyano-7-(1-methyl-1H-pyrazol- 4-yl)imidazo[1,2-a]pyridin-5-yl)pyri- din-2-yl)-2,6-diazaspiro[3.3]heptane- 2-carboxylate | MS-ESI (m/z): 497 [M + 1]⁺ |
| 4 | 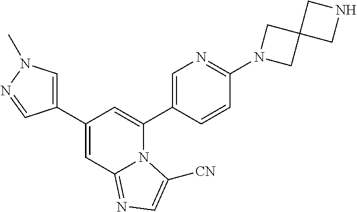 [SMILES: Cn1cc(-c2cc(-c3ccc(N4CC5(CNC5)C4)nc3)n3c(C#N)cnc3c2)cn1] | 5-(6-(2,6-diazaspiro[3.3]heptan-2-yl) pyridin-3-yl)-7-(1-methyl-1H-pyrazol- 4-yl)imidazo[1,2-a]pyridine-3-car- bonitrile | MS-ESI (m/z): 397 [M + 1]⁺ |
| 5 | 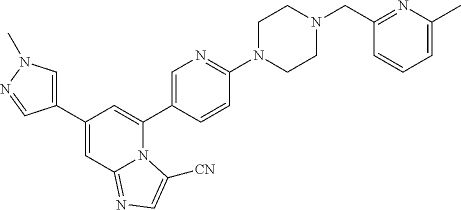 [SMILES: Cc1cccc(CN2CCN(c3ccc(-c4cc(-c5cnn(C)c5)cc5ncc(C#N)n45)cn3)CC2)n1] | 7-(1-methyl-1H-pyrazol-4-yl)-5-(6- (4-((6-methylpyridin-2-yl)methyl)pi- perazin-1-yl)pyridin-3-yl)imidazo[1,2- a]pyridine-3-carbonitrile | MS-ESI (m/z): 490 [M + 1]⁺ |
| 6 | 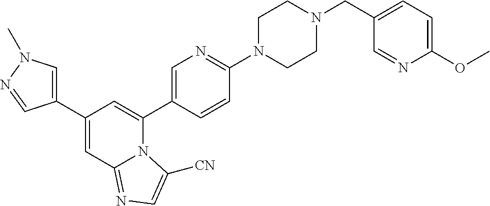 [SMILES: COc1ccc(CN2CCN(c3ccc(-c4cc(-c5cnn(C)c5)cc5ncc(C#N)n45)cn3)CC2)cn1] | 5-(6-(4-((6-methoxypyridin-3-yl)meth- yl)piperazin-1-yl)pyridin-3-yl)-7-(1- methyl-1H-pyrazol-4-yl)imidazo[1, 2-a]pyridine-3-carbonitrile | MS-ESI (m/z): 506 [M + 1]⁺ |
| 7 | 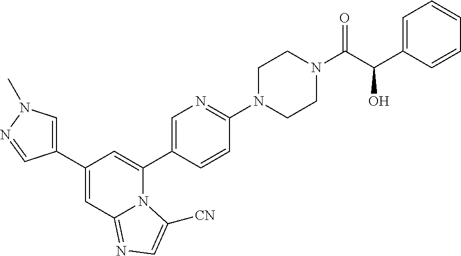 [SMILES: Cn1cc(-c2cc(-c3ccc(N4CCN(C(=O)[C@H](O)c5ccccc5)CC4)nc3)n3c(C#N)cnc3c2)cn1] | (R)-5-(6-(4-(2-hydroxy-2-phenylace- tyl)piperazin-1-yl)pyridin-3-yl)-7-(1- methyl-1H-pyrazol-4-yl)imidazo[1,2- a]pyridine-3-carbonitrile | MS-ESI (m/z): 519 [M + 1]⁺ |
| 8 | 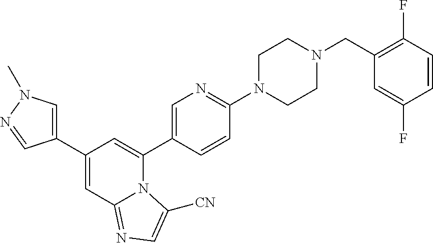 [SMILES: Cn1cc(-c2cc(-c3ccc(N4CCN(Cc5cc(F)ccc5F)CC4)nc3)n3c(C#N)cnc3c2)cn1] | 5-(6-(4-(2,5-difluorobenzyl)piperazin- 1-yl)pyridin-3-yl)-7-(1-methyl-1H- pyrazol-4-yl)imidazo[1,2-a]pyridine- 3-carbonitrile | MS-ESI (m/z): 511 [M + 1]⁺ |
| 9 | 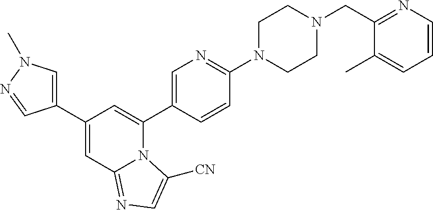 [SMILES: Cc1cccnc1CN1CCN(c2ccc(-c3cc(-c4cnn(C)c4)cc4ncc(C#N)n34)cn2)CC1] | 7-(1-methyl-1H-pyrazol-4-yl)-5-(6- (4-((3-methylpyridin-2-yl)methyl)pi- perazin-1-yl)pyridin-3-yl)imidazo[1,2- a]pyridine-3-carbonitrile | MS-ESI (m/z): 490 [M + 1]⁺ |
| 10 | 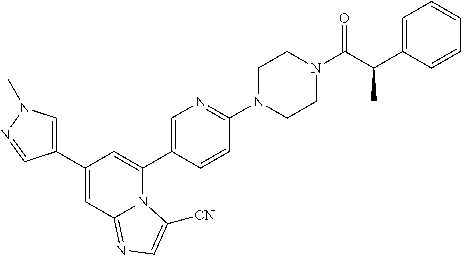 [SMILES: C[C@@H](C(=O)N1CCN(c2ccc(-c3cc(-c4cnn(C)c4)cc4ncc(C#N)n34)cn2)CC1)c1ccccc1] | (R)-7-(1-methyl-1H-pyrazol-4-yl)-5- (6-(4-(2-phenylpropanoyl)piperazin- 1-yl)pyridin-3-yl)imidazo[1,2-a]pyri- dine-3-carbonitrile | MS-ESI (m/z): 517 [M + 1]⁺ |
| 11 | 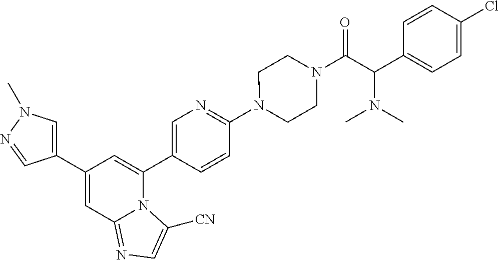 [SMILES: CN(C)C(C(=O)N1CCN(c2ccc(-c3cc(-c4cnn(C)c4)cc4ncc(C#N)n34)cn2)CC1)c1ccc(Cl)cc1] | 5-(6-(4-(2-(4-chlorophenyl)-2-(dimeth- ylamino)acetyl)piperazin-1-yl)pyri- din-3-yl)-7-(1-methyl-1H-pyrazol-4- yl)imidazo[1,2-a]pyridine-3-carboni- trile | MS-ESI (m/z): 580 [M + 1]⁺ |
| 12 | 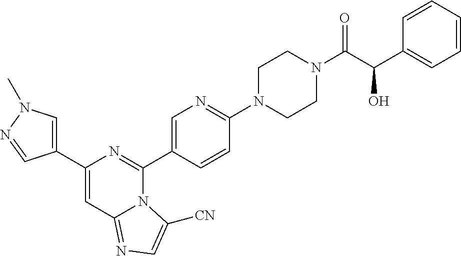 [SMILES: Cn1cc(-c2cc3ncc(C#N)n3c(-c3ccc(N4CCN(C(=O)[C@H](O)c5ccccc5)CC4)nc3)n2)cn1] | (R)-5-(6-(4-(2-hydroxy-2-phenylace- tyl)piperazin-1-yl)pyridin-3-yl)-7-(1- methyl-1H-pyrazol-4-yl)imidazo-[1,2- c]pyrimidine-3-carbonitrile | MS-ESI (m/z): 520 [M + 1]⁺ |
| 13 | 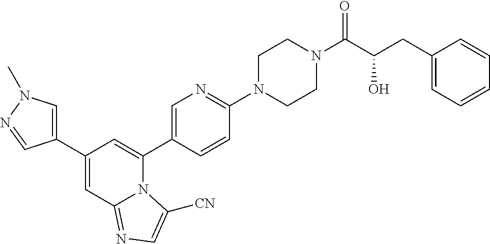 [SMILES: Cn1cc(-c2cc(-c3ccc(N4CCN(C(=O)[C@@H](O)Cc5ccccc5)CC4)nc3)n3c(C#N)cnc3c2)cn1] | (S)-5-(6-(4-(2-hydroxy-3-phenylpro- panoyl)piperazin-1-yl)pyridin-3-yl)-7- (1-methyl-1H-pyrazol-4-yl)imidazo [1,2-a]pyridine-3-carbonitrile | MS-ESI (m/z): 533 [M + 1]⁺ |
| 14 | 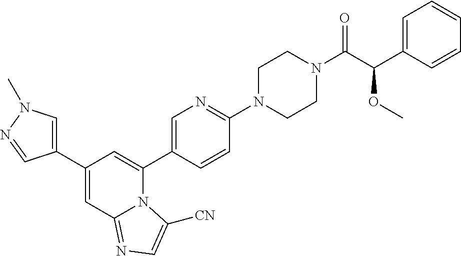 [SMILES: CO[C@@H](C(=O)N1CCN(c2ccc(-c3cc(-c4cnn(C)c4)cc4ncc(C#N)n34)cn2)CC1)c1ccccc1] | (R)-5-(6-(4-(2-methoxy-2-phenylace- tyl)piperazin-1-yl)pyridin-3-yl)-7-(1- methyl-1H-pyrazol-4-yl)imidazo[1,2- a]pyridine-3-carbonitrile | MS-ESI (m/z): 533 [M + 1]⁺ |
| 15 | 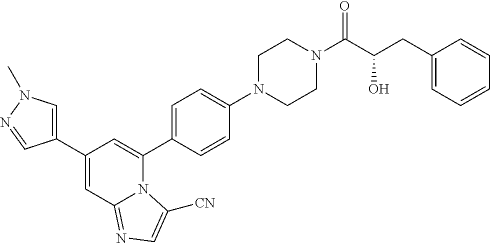 [SMILES: Cn1cc(-c2cc(-c3ccc(N4CCN(C(=O)[C@@H](O)Cc5ccccc5)CC4)cc3)n3c(C#N)cnc3c2)cn1] | (S)-5-(4-(4-(2-hydroxy-3-phenylpro- panoyl)piperazin-1-yl)phenyl)-7-(1- methyl-1H-pyrazol-4-yl)imidazo[1,2-a] pyridine-3-carbonitrile | MS-ESI (m/z): 532 [M + 1]⁺ |
| 16 | 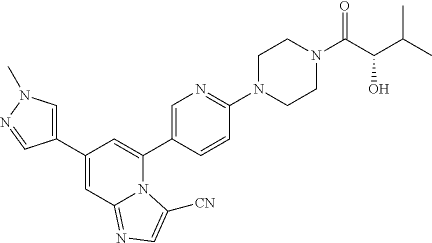 [SMILES: CC(C)[C@H](O)C(=O)N1CCN(c2ccc(-c3cc(-c4cnn(C)c4)cc4ncc(C#N)n34)cn2)CC1] | (S)-5-(6-(4-(2-hydroxy-3-methylbuta- noyl)piperazin-1-yl)pyridin-3-yl)-7- (1-methyl-1H-pyrazol-4-yl)imidazo[1, 2-a]pyridine-3-carbonitrile | MS-ESI (m/z): 485 [M + 1]⁺ |
| 17 | 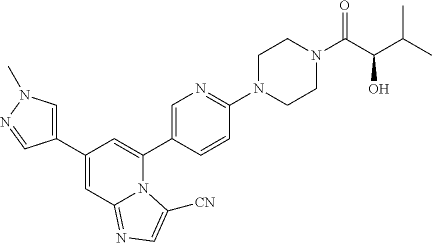 [SMILES: CC(C)[C@@H](O)C(=O)N1CCN(c2ccc(-c3cc(-c4cnn(C)c4)cc4ncc(C#N)n34)cn2)CC1] | (R)-5-(6-(4-(2-hydroxy-3-methylbuta- noyl)piperazin-1-yl)pyridin-3-yl)-7- (1-methyl-1H-pyrazol-4-yl)imidazo [1,2-a]pyridine-3-carbonitrile | MS ESI (m/z): 485 [M + 1]⁺ |
| 18 | 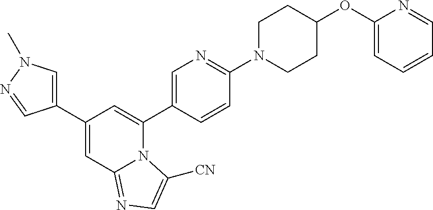 [SMILES: Cn1cc(-c2cc(-c3ccc(N4CCC(Oc5ccccn5)CC4)nc3)n3c(C#N)cnc3c2)cn1] | 7-(1-methyl-1H-pyrazol-4-yl)-5-(6- (4-(pyridin-2-yloxy)piperidin-1-yl)pyr- idin-3-yl)imidazo[1,2-a]pyridine-3- carbonitrile | MS-ESI (m/z): 477 [M + 1]⁺ |
| 19 | 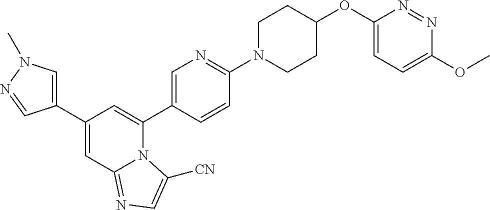 [SMILES: COc1ccc(OC2CCN(c3ccc(-c4cc(-c5cnn(C)c5)cc5ncc(C#N)n45)cn3)CC2)nn1] | 5-(6-(4-((6-methoxypyridazin-3-yl) oxy)piperidin-1-yl)pyridin-3-yl)-7-(1- methyl-1H-pyrazol-4-yl)imidazo[1,2- a]pyridine-3-carbonitrile | MS-ESI (m/z): 508 [M + 1]⁺ |
| 20 | 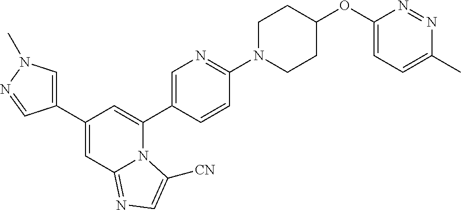 [SMILES: Cc1ccc(OC2CCN(c3ccc(-c4cc(-c5cnn(C)c5)cc5ncc(C#N)n45)cn3)CC2)nn1] | 7-(1-methyl-1H-pyrazol-4-yl)-5-(6- (4-((6-methylpyridazin-3-yl)oxy)piper- idin-1-yl)pyridin-3-yl)imidazo[1,2- a]pyridine-3-carbonitrile | MS-ESI (m/z): 492 [M + 1]⁺ |
| 21 | 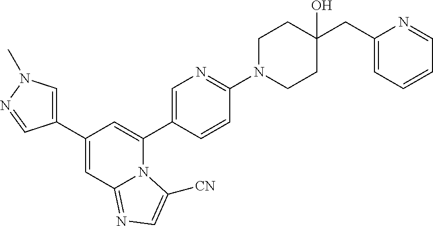 [SMILES: Cn1cc(-c2cc(-c3ccc(N4CCC(O)(Cc5ccccn5)CC4)nc3)n3c(C#N)cnc3c2)cn1] | 5-(6-(4-hydroxy-4-(pyridin-2-ylmeth- yl)piperidin-1-yl)pyridin-3-yl)-7-(1- methyl-1H-pyrazol-4-yl)imidazo[1,2- a]pyridine-3-carbonitrile | MS-ESI (m/z): 491 [M + 1]⁺ |
| 22 | 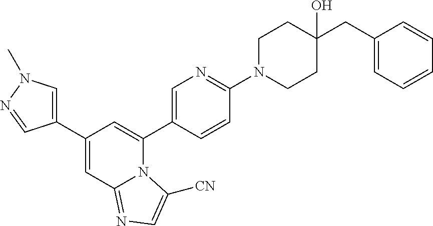 [SMILES: Cn1cc(-c2cc(-c3ccc(N4CCC(O)(Cc5ccccc5)CC4)nc3)n3c(C#N)cnc3c2)cn1] | 5-(6-(4-benzyl-4-hydroxypiperidin-1- yl)pyridin-3-yl)-7-(1-methyl-1H-pyr- azol-4-yl)imidazo[1,2-a]pyridine-3- carbonitrile | MS-ESI (m/z): 490 [M + 1]⁺ |
| 23 | 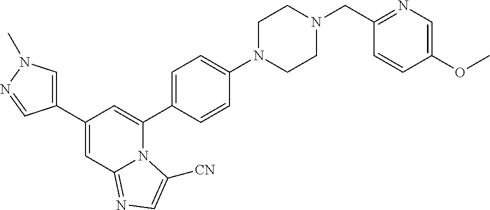 [SMILES: COc1ccc(CN2CCN(c3ccc(-c4cc(-c5cnn(C)c5)cc5ncc(C#N)n45)cc3)CC2)nc1] | 5-(4-(4-((5-methoxypyridin-2-yl)meth- yl)piperazin-1-yl)phenyl)-7-(1-meth- yl-1H-pyrazol-4-yl)imidazo[1,2-a] pyridine-3-carbonitrile | MS-ESI (m/z): 505 [M + 1]⁺ |
| 24 | 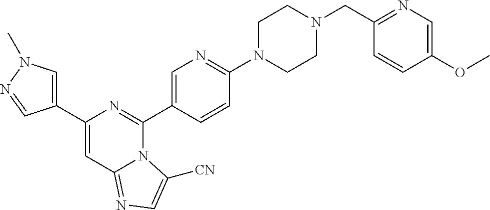 [SMILES: COc1ccc(CN2CCN(c3ccc(-c4nc(-c5cnn(C)c5)cc5ncc(C#N)n45)cn3)CC2)nc1] | 5-(6-(4-((5-methoxypyridin-2-yl)meth- yl)piperazin-1-yl)pyridin-3-yl)-7-(1- methyl-1H-pyrazol-4-yl)imidazo[1, 2-c]pyrimidine-3-carbonitrile | MS-ESI (m/z): 507 [M + 1]⁺ |
| 25 | 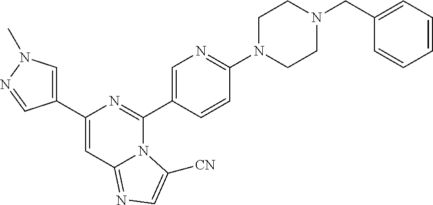 [SMILES: Cn1cc(-c2cc3ncc(C#N)n3c(-c3ccc(N4CCN(Cc5ccccc5)CC4)nc3)n2)cn1] | 5-(6-(4-benzylpiperazin-1-yl)pyridin- 3-yl)-7-(1-methyl-1H-pyrazol-4-yl) imidazo[1,2-c]pyrimidine-3-carbonitrile | MS-ESI (m/z): 476 [M + 1]⁺ |
| 26 | 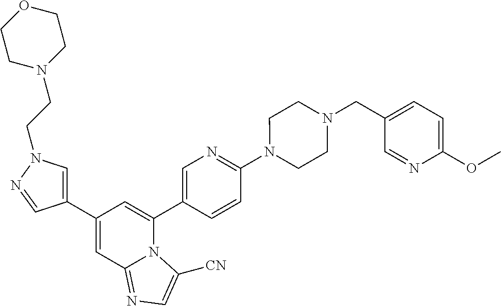 [SMILES: COc1ccc(CN2CCN(c3ccc(-c4cc(-c5cnn(CCN6CCOCC6)c5)cc5ncc(C#N)n45)cn3)CC2)cn1] | 5-(6-(4-((6-methoxypyridin-3-yl)meth- yl)piperazin-1-yl)pyridin-3-yl)-7-(1- (2-morpholinoethyl)-1H-pyrazol-4- yl)imidazo[1,2-a]pyridine-3-carbonitrile | MS-ESI (m/z): 605 [M + 1]⁺ |
| 27 | 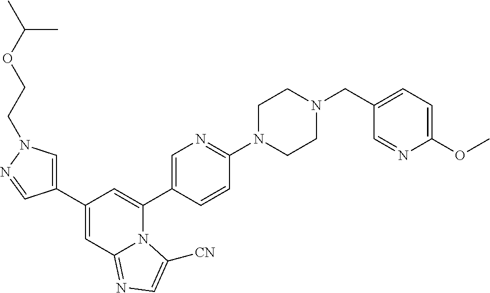 [SMILES: COc1ccc(CN2CCN(c3ccc(-c4cc(-c5cnn(CCOC(C)C)c5)cc5ncc(C#N)n45)cn3)CC2)cn1] | 7-(1-(2-isopropoxyethyl)-1H-pyrazol- 4-yl)-5-(6-(4-((6-methoxypyridin-3- yl)methyl)piperazin-1-yl)pyridin-3-yl) imidazo[1,2-a]pyridine-3-carbonitrile | MS-ESI (m/z): 578 [M + 1]⁺ |
| 28 | 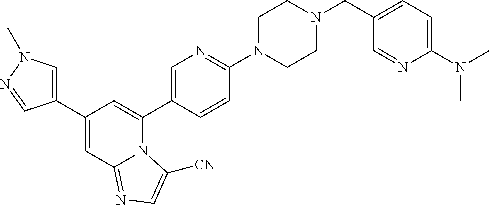 [SMILES: CN(C)c1ccc(CN2CCN(c3ccc(-c4cc(-c5cnn(C)c5)cc5ncc(C#N)n45)cn3)CC2)cn1] | 5-(6-(4-((6-(dimethylamino)pyridin- 3-yl)methyl)piperazin-1-yl)pyridin-3- yl)-7-(1-methyl-1H-pyrazol-4-yl)im- idazo[1,2-a]pyridine-3-carbonitrile | MS-ESI (m/z): 519 [M + 1]⁺ |
| 29 | 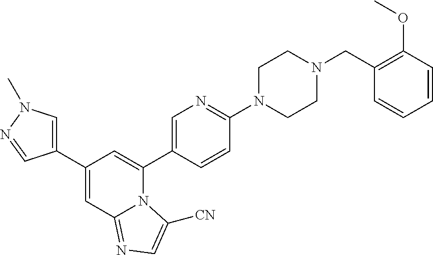 [SMILES: COc1ccccc1CN1CCN(c2ccc(-c3cc(-c4cnn(C)c4)cc4ncc(C#N)n34)cn2)CC1] | 5-(6-(4-(2-methoxybenzyl)piperazin- 1-yl)pyridin-3-yl)-7-(1-methyl-1H- pyrazol-4-yl)imidazo[1,2-a]pyridine-3- carbonitrile | MS-ESI (m/z): 505 [M + 1]⁺ |
| 30 | 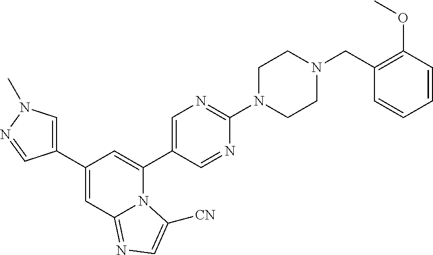 [SMILES: COc1ccccc1CN1CCN(c2ncc(-c3cc(-c4cnn(C)c4)cc4ncc(C#N)n34)cn2)CC1] | 5-(2-(4-(2-methoxybenzyl)piperazin- 1-yl)pyrimidin-5-yl)-7-(1-methyl-1H- pyrazol-4-yl)imidazo[1,2-a]pyridine- 3-carbonitrile | MS-ESI (m/z): 506 [M + 1]⁺ |
| 31 | 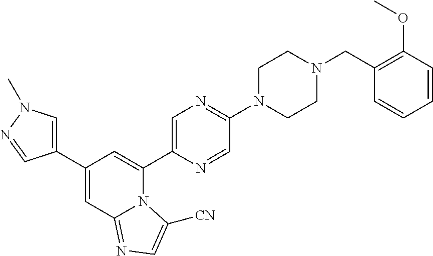 [SMILES: COc1ccccc1CN1CCN(c2cnc(-c3cc(-c4cnn(C)c4)cc4ncc(C#N)n34)cn2)CC1] | 5-(5-(4-(2-methoxybenzyl)piperazin- 1-yl)pyrazin-2-yl)-7-(1-methyl-1H- pyrazol-4-yl)imidazo[1,2-a]pyridine-3- carbonitrile | MS-ESI (m/z): 506 [M + 1]⁺ |
| 32 | 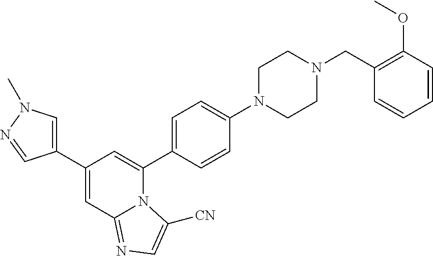 [SMILES: COc1ccccc1CN1CCN(c2ccc(-c3cc(-c4cnn(C)c4)cc4ncc(C#N)n34)cc2)CC1] | 5-(4-(4-(2-methoxybenzyl)piperazin- 1-yl)phenyl)-7-(1-methyl-1H-pyrazol- 4-yl)imidazo[1,2-a]pyridine-3-car- bonitrile | MS-ESI (m/z): 504 [M + 1]⁺ |
| 33 | 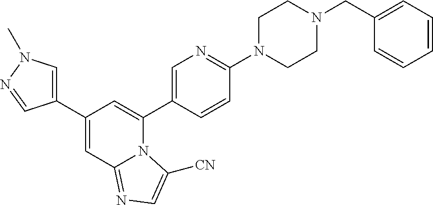 [SMILES: Cn1cc(-c2cc(-c3ccc(N4CCN(Cc5ccccc5)CC4)nc3)n3c(C#N)cnc3c2)cn1] | 5-(6-(4-benzylpiperazin-1-yl)pyridin- 3-yl)-7-(1-methyl-1H-pyrazol-4-yl) imidazo[1,2-a]pyridine-3-carbonitrile | MS-ESI (m/z): 475 [M + 1]⁺ |
| 34 | 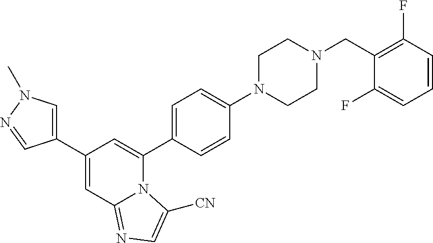 [SMILES: Cn1cc(-c2cc(-c3ccc(N4CCN(Cc5c(F)cccc5F)CC4)cc3)n3c(C#N)cnc3c2)cn1] | 5-(4-(4-(2,6-difluorobenzyl)piperazin- 1-yl)phenyl)-7-(1-methyl-1H-pyr- azol-4-yl)imidazo[1,2-a]pyridine-3-car- bonitrile | MS-ESI (m/z): 510 [M + 1]⁺ |
| 35 | 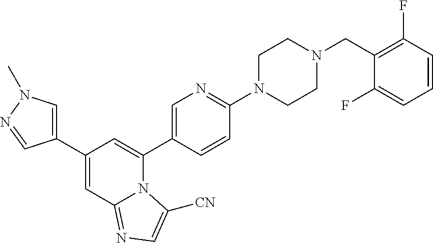 [SMILES: Cn1cc(-c2cc(-c3ccc(N4CCN(Cc5c(F)cccc5F)CC4)nc3)n3c(C#N)cnc3c2)cn1] | 5-(6-(4-(2,6-difluorobenzyl)piperazin- 1-yl)pyridin-3-yl)-7-(1-methyl-1H- pyrazol-4-yl)imidazo[1,2-a]pyridine- 3-carbonitrile | MS-ESI (m/z): 511 [M + 1]⁺ |
| 36 | 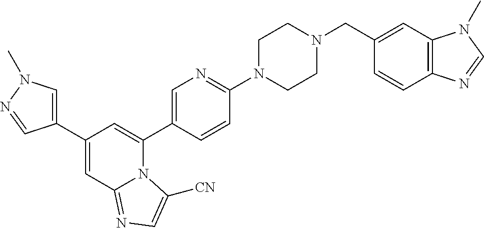 [SMILES: Cn1cc(-c2cc(-c3ccc(N4CCN(Cc5ccc6ncn(C)c6c5)CC4)nc3)n3c(C#N)cnc3c2)cn1] | 5-(6-(4-((1-methyl-1H-benzo[d]imid- azol-6-yl)methyl)piperazin-1-yl)pyri- din-3-yl)-7-(1-methyl-1H-pyrazol-4- yl)imidazo[1,2-a]pyridine-3-carboni- trile | MS-ESI (m/z): 529 [M + 1]⁺ |
| 37 | 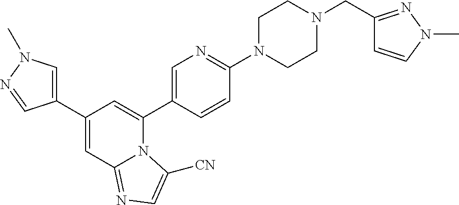 [SMILES: Cn1cc(-c2cc(-c3ccc(N4CCN(Cc5ccn(C)n5)CC4)nc3)n3c(C#N)cnc3c2)cn1] | 5-(6-(4-((1-methyl-1H-pyrazol-3-yl) methyl)piperazin-1-yl)pyridin-3-yl)- 7-(1-methyl-1H-pyrazol-4-yl)imidazo [1,2-a]pyridine-3-carbonitrile | MS-ESI (m/z): 479 [M + 1]⁺ |
| 38 | 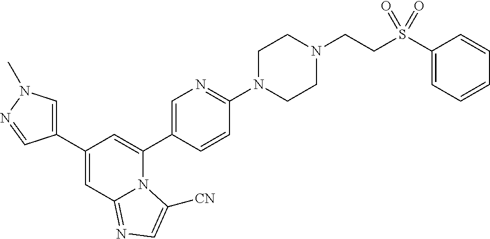 [SMILES: Cn1cc(-c2cc(-c3ccc(N4CCN(CCS(=O)(=O)c5ccccc5)CC4)nc3)n3c(C#N)cnc3c2)cn1] | 7-(1-methyl-1H-pyrazol-4-yl)-5-(6- (4-(2-(phenylsulfonyl)ethyl)piperazin- 1-yl)pyridin-3-yl)imidazo[1,2-a]pyr- idine-3-carbonitrile | MS-ESI (m/z): 553 [M + 1]⁺ |
| 39 | 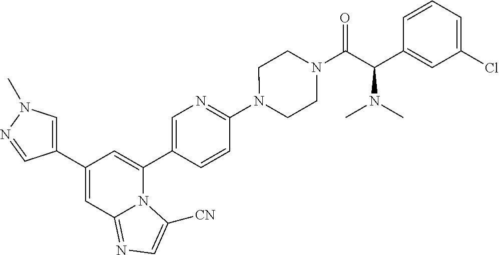 [SMILES: CN(C)[C@@H](C(=O)N1CCN(c2ccc(-c3cc(-c4cnn(C)c4)cc4ncc(C#N)n34)cn2)CC1)c1cccc(Cl)c1] | (R)-5-(6-(4-(2-(3-chlorophenyl)-2- (dimethylamino)acetyl)piperazin-1-yl) pyridin-3-yl)-7-(1-methyl-1H-pyrazol- 4-yl)imidazo[1,2-a]pyridine-3-car- bonitrile | MS-ESI (m/z): 580 [M + 1]⁺ |
| 40 | 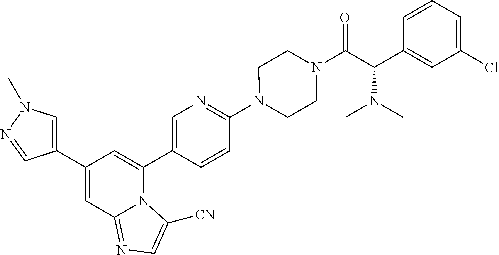 [SMILES: CN(C)[C@H](C(=O)N1CCN(c2ccc(-c3cc(-c4cnn(C)c4)cc4ncc(C#N)n34)cn2)CC1)c1cccc(Cl)c1] | (S)-5-(6-(4-(2-(3-chlorophenyl)-2-(di- methylamino)acetyl)piperazin-1-yl) pyridin-3-yl)-7-(1-methyl-1H-pyrazol- 4-yl)imidazo[1,2-a]pyridine-3-car- bonitrile | MS-ESI (m/z): 580 [M + 1]⁺ |
| 41 | 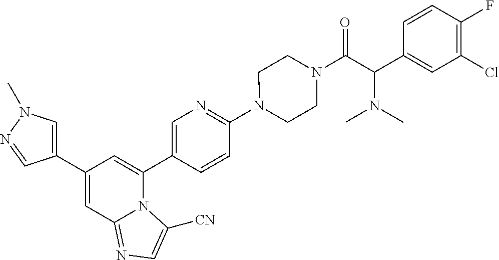 [SMILES: CN(C)C(C(=O)N1CCN(c2ccc(-c3cc(-c4cnn(C)c4)cc4ncc(C#N)n34)cn2)CC1)c1ccc(F)c(Cl)c1] | 5-(6-(4-(2-(3-chloro-4-fluorophenyl)- 2-(dimethylamino)acetyl)piperazin- 1-yl)pyridin-3-yl)-7-(1-methyl-1H- pyrazol-4-yl)imidazo[1,2-a]pyridine-3- carbonitrile | MS-ESI (m/z): 598 [M + 1]⁺ |
| 42 | 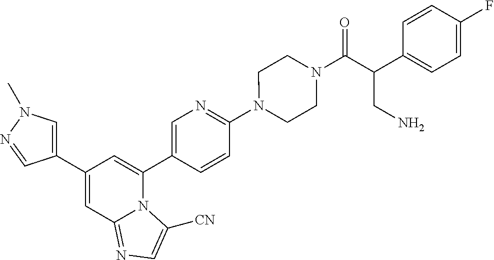 [SMILES: Cn1cc(-c2cc(-c3ccc(N4CCN(C(=O)C(CN)c5ccc(F)cc5)CC4)nc3)n3c(C#N)cnc3c2)cn1] | 5-(6-(4-(3-amino-2-(4-fluorophenyl) propanoyl)piperazin-1-yl)pyridin-3- yl)-7-(1-methyl-1H-pyrazol-4-yl)im- idazo[1,2-a]pyridine-3-carbonitrile | MS-ESI (m/z): 550 [M + 1]⁺ |
| 43 | 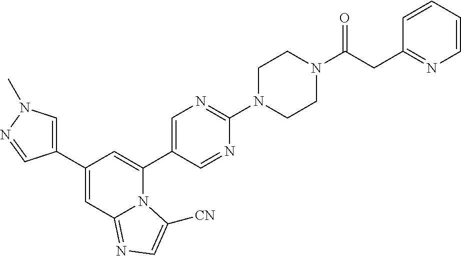 [SMILES: Cn1cc(-c2cc(-c3cnc(N4CCN(C(=O)Cc5ccccn5)CC4)nc3)n3c(C#N)cnc3c2)cn1] | 7-(1-methyl-1H-pyrazol-4-yl)-5-(2- (4-(2-(pyridin-2-yl)acetyl)piperazin-1- yl)pyrimidin-5-yl)imidazo[1,2-a] pyridine-3-carbonitrile | MS-ESI (m/z): 505 [M + 1]⁺ |
| 44 | 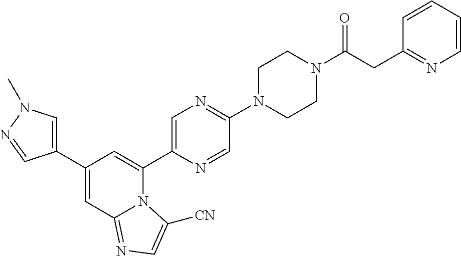 [SMILES: Cn1cc(-c2cc(-c3cnc(N4CCN(C(=O)Cc5ccccn5)CC4)cn3)n3c(C#N)cnc3c2)cn1] | 7-(1-methyl-1H-pyrazol-4-yl)-5-(5- (4-(2-(pyridin-2-yl)acetyl)piperazin-1- yl)pyrazin-2-yl)imidazo[1,2-a]pyri- dine-3-carbonitrile | MS-ESI (m/z): 505 [M + 1]⁺ |
| 45 | 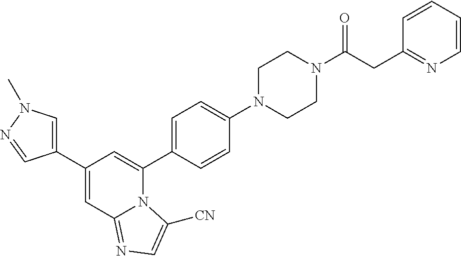 [SMILES: Cn1cc(-c2cc(-c3ccc(N4CCN(C(=O)Cc5ccccn5)CC4)cc3)n3c(C#N)cnc3c2)cn1] | 7-(1-methyl-1H-pyrazol-4-yl)-5-(4- (4-(2-(pyridin-2-yl)acetyl)piperazin-1- yl)phenyl)imidazo[1,2-a]pyridine-3- carbonitrile | MS-ESI (m/z): 503 [M + 1]⁺ |
| 46 | 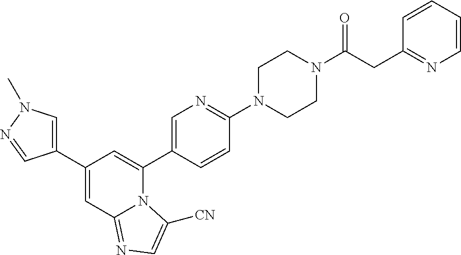 [SMILES: Cn1cc(-c2cc(-c3ccc(N4CCN(C(=O)Cc5ccccn5)CC4)nc3)n3c(C#N)cnc3c2)cn1] | 7-(1-methyl-1H-pyrazol-4-yl)-5-(6- (4-(2-(pyridin-2-yl)acetyl)piperazin-1- yl)pyridin-3-yl)imidazo[1,2-a]pyri- dine-3-carbonitrile | MS-ESI (m/z): 504 [M + 1]⁺ |
| 47 | 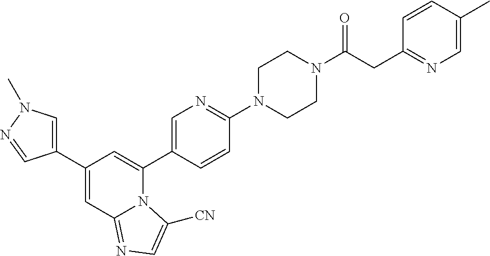 [SMILES: Cc1ccc(CC(=O)N2CCN(c3ccc(-c4cc(-c5cnn(C)c5)cc5ncc(C#N)n45)cn3)CC2)nc1] | 7-(1-methyl-1H-pyrazol-4-yl)-5-(6- (4-(2-(5-methylpyridin-2-yl)acetyl)pi- perazin-1-yl)pyridin-3-yl)imidazo[1, 2-a]pyridine-3-carbonitrile | MS-ESI (m/z): 518 [M + 1]⁺ |
| 48 | 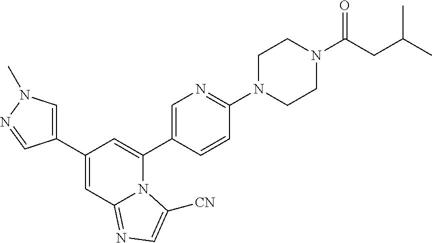 [SMILES: CC(C)CC(=O)N1CCN(c2ccc(-c3cc(-c4cnn(C)c4)cc4ncc(C#N)n34)cn2)CC1] | 7-(1-methyl-1H-pyrazol-4-yl)-5-(6- (4-(3-methylbutanoyl)piperazin-1-yl) pyridin-3-yl)imidazo[1,2-a]pyridine- 3-carbonitrile | MS-ESI (m/z): 469 [M + 1]⁺ |
| 49 | 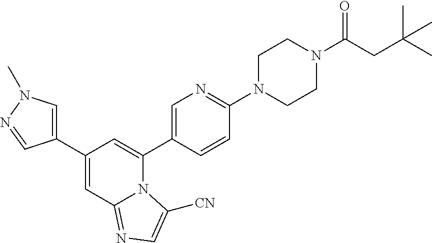 [SMILES: Cn1cc(-c2cc(-c3ccc(N4CCN(C(=O)CC(C)(C)C)CC4)nc3)n3c(C#N)cnc3c2)cn1] | 5-(6-(4-(3,3-dimethylbutanoyl)piper- azin-1-yl)pyridin-3-yl)-7-(1-methyl- 1H-pyrazol-4-yl)imidazo[1,2-a]pyri- dine-3-carbonitrile | MS-ESI (m/z): 483 [M + 1]⁺ |
| 50 | 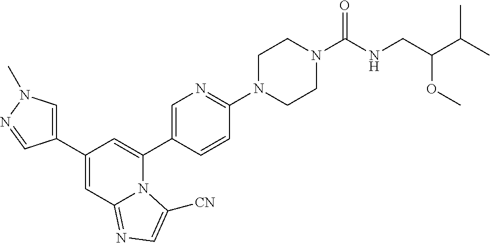 [SMILES: COC(CNC(=O)N1CCN(c2ccc(-c3cc(-c4cnn(C)c4)cc4ncc(C#N)n34)cn2)CC1)C(C)C] | 4-(5-(3-cyano-7-(1-methyl-1H-pyrazol- 4-yl)imidazo[1,2-a]pyridin-5-yl) pyridin-2-yl)-N-(2-methoxy-3-methyl- butyl)piperazine-1-carboxamide | MS-ESI (m/z): 528 [M + 1]⁺ |
| 51 | 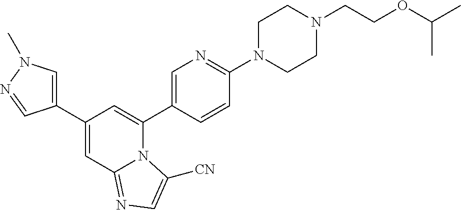 [SMILES: CC(C)OCCN1CCN(c2ccc(-c3cc(-c4cnn(C)c4)cc4ncc(C#N)n34)cn2)CC1] | 5-(6-(4-(2-isopropoxyethyl)piperazin- 1-yl)pyridin-3-yl)-7-(1-methyl-1H- pyrazol-4-yl)imidazo[1,2-a]pyridine- 3-carbonitrile | MS-ESI (m/z): 471 [M + 1]⁺ |
| 52 | 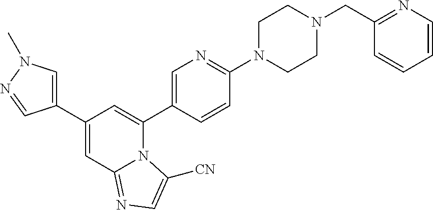 [SMILES: Cn1cc(-c2cc(-c3ccc(N4CCN(Cc5ccccn5)CC4)nc3)n3c(C#N)cnc3c2)cn1] | 7-(1-methyl-1H-pyrazol-4-yl)-5-(6- (4-(pyridin-2-ylmethyl)piperazin-1-yl) pyridin-3-yl)imidazo[1,2-a]pyridine- 3-carbonitrile | MS-ESI (m/z): 476 [M + 1]⁺ |
| 53 | 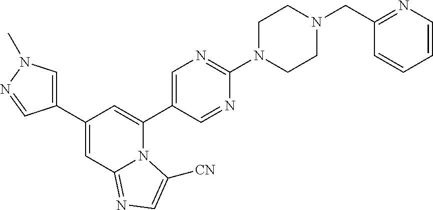 [SMILES: Cn1cc(-c2cc(-c3cnc(N4CCN(Cc5ccccn5)CC4)nc3)n3c(C#N)cnc3c2)cn1] | 7-(1-methyl-1H-pyrazol-4-yl)-5-(2- (4-(pyridin-2-ylmethyl)piperazin-1-yl) pyrimidin-5-yl)imidazo[1,2-a]pyridine- 3-carbonitrile | MS-ESI (m/z): 477 [M + 1]⁺ |
| 54 | 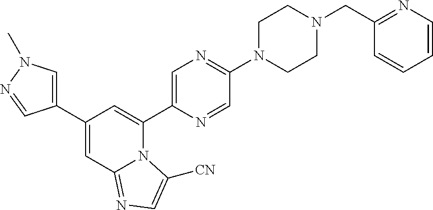 [SMILES: Cn1cc(-c2cc(-c3cnc(N4CCN(Cc5ccccn5)CC4)cn3)n3c(C#N)cnc3c2)cn1] | 7-(1-methyl-1H-pyrazol-4-yl)-5-(5- (4-(pyridin-2-ylmethyl)piperazin-1-yl) pyrazin-2-yl)imidazo[1,2-a]pyridine- 3-carbonitrile | MS-ESI (m/z): 477 [M + 1]⁺ |
| 55 | 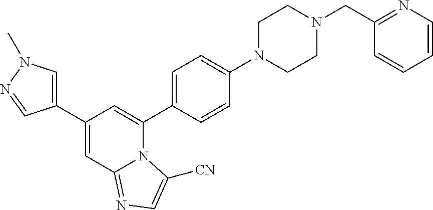 [SMILES: Cn1cc(-c2cc(-c3ccc(N4CCN(Cc5ccccn5)CC4)cc3)n3c(C#N)cnc3c2)cn1] | 7-(1-methyl-1H-pyrazol-4-yl)-5-(4- (4-(pyridin-2-ylmethyl)piperazin-1-yl) phenyl)imidazo[1,2-a]pyridine-3-car- bonitrile | MS-ESI (m/z): 475 [M + 1]⁺ |

### Example 56

### 7-(2-Hydroxy-2-methylpropoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (56)

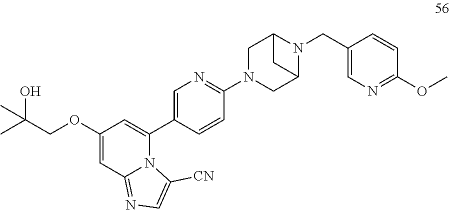 [SMILES: COc1ccc(CN2C3CC2CN(c2ccc(-c4cc(OCC(C)(C)O)cc5ncc(C#N)n45)cn2)C3)cn1]

### 5-(6-Fluoropyridin-3-yl)-7-(2-hydroxy-2-methylpropoxy)imidazo[1,2-a]pyridine-3-carbonitrile (56a)

A mixture of 5-(6-fluoropyridin-3-yl)-7-hydroxyimidazo[1,2-a]pyridine-3-carbonitrile (1e) (25.0 mg, 0.100 mmol), 2,2-dimethyloxirane (72.0 mg, 1.00 mmol) and K₂CO₃ (41.0 mg, 0.300 mmol) in DMF (1 mL) was stirred at 80° C. in a sealed tube for overnight. The mixture was cooled to RT and diluted with H₂O, extracted with BA (4×50 mL). The organic phase was washed with H₂O, brine, dried over Na₂SO₄ and concentrated. The residue was purified by column chromatography on silica gel, eluting with DCM/MeOH (100:1) to give the title compound 5-(6-fluoropyridin-3-yl)-7-(2-hydroxy-2-methylpropoxy)imidazo[1,2-a]pyridine-3-carbonitrile (56a). MS-ESI (m/z): 327 [M+1]⁺.

### 7-(2-Hydroxy-2-methylpropoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (56)

The title compound 7-(2-hydroxy-2-methylpropoxy)-5-(6-(6-((6-methoxypyridin-3-yl)methyl)-3,6-diazabicyclo[3.1.1]heptan-3-yl)pyridin-3-yl)imidazo[1,2-a]pyri dine-3-carbonitrile (56) was prepared according to the synthetic method of 1 by replacing 5-(6-fluoropyridin-3-yl)-7-(1-methyl-1H-pyrazol-4-yl)imidazo[1,2-a]pyridine-3-carbonitrile (1g), tert-butyl piperazine-1-carboxylate and 4-fluorobenzaldehyde with 5-(6-fluoropyridin-3-yl)-7-(2-hydroxy-2-methylpropoxy)imidazo[1,2-a]pyridine-3-carbonitrile (56a), tert-butyl 3,6-diazabicyclo[3.1.1]heptane-6-carboxylate and 6-methoxynicotinaldehyde. MS-ESI (m/z): 526 [M+1]+.

### Example 57A/B

### 7-((1-Hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57A and 57B)

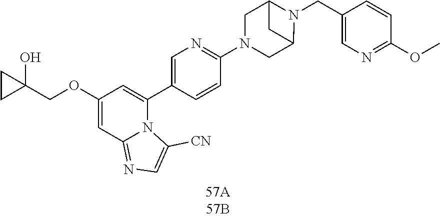 [SMILES: COc1ccc(CN2C3CC2CN(c2ccc(-c4cc(OCC5(O)CC5)cc5ncc(C#N)n45)cn2)C3)cn1]

### Methyl 1-((tert-butyldimethylsilyl)oxy)cyclopropane-1-carboxylate (57a)

To a solution of methyl 1-hydroxycyclopropane-1-carboxylate (2.32 g, 20.0 mmol) and imidazole (1.43 g, 21.0 mmol) in DMF at 0° C. was added TBSCl (3.17 g, 21.0 mmol). Then the mixture was stirred at RT for overnight. The mixture was diluted with H₂O, extracted with Et₂O, washed with H₂O, brine, dried over Na₂SO₄ and concentrated to give the title compound methyl 1-((tert-butyldimethylsilyl)oxy)cyclopropane-1-carboxylate (57a). MS-ESI (m/z): 231 [M+1]+.

### (1-((Tert-butyldimethylsilyl)oxy)cyclopropyl)methanol (57b)

To a solution of 1-((tert-butyldimethylsilyl)oxy)cyclopropane-1-carboxylate (57a) (4.60 g, 20.0 mmol) in THF at 0° C. was added DIBAL (1.5 M solution in toluene, 33.3 mL, 50.0 mmol) dropwise and stirred at RT for 1.5 h under N₂. The mixture was cooled to 0° C. and quenched with 0.5 M sodium potassium tartrate tetrahydrate aqueous solution (30 mL). The mixture was diluted with Et₂O and stirred at RT for 15 min. Filtered, the filtrate was washed with H₂O, brine dried over Na₂SO₄ and concentrated to give the title compound (1-((tert-butyldimethylsilyl)oxy)cyclopropyl)methanol (57b). MS-ESI (m/z): 203 [M+1]+.

### 7-((1-((Tert-butyldimethylsilyl)oxy)cyclopropyl)methoxy)-5-(6-fluoropyridin-3-yl) imidazo[1,2-a]pyridine-3-carbonitrile (57c)

A mixture of 5-(6-fluoropyridin-3-yl)-7-hydroxyimidazo[1,2-a]pyridine-3-carbonitrile (1e) (50.0 mg, 0.196 mmol), (1-((tert-butyldimethylsilyl)oxy)cyclopropyl)methanol (57b) (120 mg, 0.594 mmol), PPh₃ (157 mg, 0.599 mmol) and DIAD (120 mg, 594 mmol) in THF/DCM (1 mL/1 mL) was stirred at RT under N₂ for overnight. The mixture was concentrated and the residue was purified by column chromatography on silica gel eluting with PE/EtOAc (5:1˜3:1) to give the title compound 7-((1-((tert-butyldimethylsilyl)oxy)cyclopropyl)methoxy)-5-(6-fluoropyridin-3-yl)imidazo[1,2-a]pyridine-3-carbonitrile (57c). MS-ESI (m/z): 439 [M+1]+.

### 5-(6-Fluoropyridin-3-yl)-7-((1-hydroxycyclopropyl)methoxy)imidazo[1,2-a]pyridine-3-carbonitrile (57d)

A mixture of 7-((1-((tert-butyldimethylsilyl)oxy)cyclopropyl)methoxy)-5-(6-fluoropyridin-3-yl) imidazo[1,2-a]pyridine-3-carbonitrile (57c) (18.0 mg, 0.041 mmol) and TBAF (1 mol/L in THF) (0.2 mL) in THF (2 mL) was stirred at RT for 1 h. The mixture was diluted with EtOAc and washed with H₂O, brine, dried over Na₂SO₄ and concentrated to give the title compound 5-(6-fluoropyridin-3-yl)-7-((1-hydroxycyclopropyl)methoxy)imidazo[1,2-a]pyridine-3-carbonitrile (57d). MS-ESI (m/z): 325 [M+1]+.

### Tert-butyl 3-(5-(3-cyano-7-((1-hydroxycyclopropyl)methoxy)imidazo[1,2-a]pyridin-5-yl)pyridin-2-yl)-3,6-diazabicyclo[3.1.1]heptane-6-carboxylate (57e)

A mixture of 5-(6-fluoropyridin-3-yl)-7-((1-hydroxycyclopropyl)methoxy) imidazo[1,2-a]pyridine-3-carbonitrile (57d) (17.0 mg, 0.0523 mmol), tert-butyl 3,6-diazabicyclo[3.1.1]heptane-6-carboxylate (16.0 mg, 0.0785 mmol) and KOAc (11.0 mg, 0.105 mmol) in DMSO (1 mL) was stirred at 80° C. for overnight. The mixture was cooled to RT and diluted with EtOAc, washed with H₂O, brine, dried over Na₂SO₄ and concentrated. The residue was purified by column chromatography on silica gel, eluting with PE/EA (1:2) to give the title compound tert-butyl 3-(5-(3-cyano-7-((1-hydroxycyclopropyl)methoxy)imidazo[1,2-a]pyridin-5-yl)pyridin-2-yl)-3,6-diazabicyclo[3.1.1]heptane-6-carboxylate (57e). MS-ESI (m/z): 503 [M+1]+.

### 5-(6-(3,6-Diazabicyclo[3.1.1]heptan-3-yl)pyridin-3-yl)-7-((1-hydroxycyclopropyl) methoxy)imidazo[1,2-a]pyridine-3-carbonitrile (57f-A) and (57f-B)

A mixture of tert-butyl 3-(5-(3-cyano-7-((1-hydroxycyclopropyl)methoxy) imidazo[1,2-a]pyridin-5-yl)pyridin-2-yl)-3,6-diazabicyclo[3.1.1]heptane-6-carboxylate (57e) (7.0 mg, 0.014 mmol) in HCl/EA (4 M, 1 mL) was stirred at RT for 1 h. The reaction solution was concentrated and give the mixture of hydrochloride of the title compound 5-(6-(3,6-diazabicyclo[3.1.1]heptan-3-yl)pyridin-3-yl)-7-((1-hydroxycyclopropyl)methoxy)imidazo[1,2-a]pyridine-3-carbonitrile (57f-A) and (57f-B). MS-ESI (m/z): 403 [M+1]⁺.

### 7-((1-Hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57A)

The title compound 7-((1-hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyrid ine-3-carbonitrile (57A) was prepared according to the synthetic method of 1 by replacing 4-fluorobenzaldehyde and 7-(1-methyl-1H-pyrazol-4-yl)-5-(6-(piperazin-1-yl)pyridin-3-yl) imidazo[1,2-a]pyridine-3-carbonitrile (li) with 6-methoxynicotinaldehyde, 5-(6-(3,6-diazabicyclo[3.1.1]heptan-3-yl)pyridine-3-yl)-7-((1-hydroxycyclopropyl)methoxy)imidazo[1,2-a]pyridine-3-carbonitrile (57f-A) and (57f-B). After purification by preparative TLC, the upper spot was separated as the title compound 7-((1-hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57A). MS-ESI (m/z): 524 [M+1]⁺.

### 7-((1-Hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57B)

The title compound 7-((1-hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57B) was prepared according to the synthetic method of 1 by replacing 4-fluorobenzaldehyde and 7-(1-methyl-1H-pyrazol-4-yl)-5-(6-(piperazin-1-yl)pyridin-3-yl) imidazo[1,2-a]pyridine-3-carbonitrile (li) with 6-methoxynicotinaldehyde, 5-(6-(3,6-diazabicyclo[3.1.1]heptan-3-yl)pyridine-3-yl)-7-((1-hydroxycyclopropyl)methoxy)imidazo[1,2-a]pyridine-3-carbonitrile (57f-A) and (57f-B). After purification by preparative TLC, the lower spot was separated as the title compound 7-((1-hydroxycyclopropyl)methoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (57B). MS-ESI (m/z): 524 [M+1]⁺.

### Example 58

### 7-(2-((Dimethyl(oxo)-λ⁶-sulfanylidene)amino)ethoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (58)

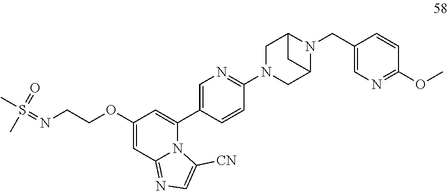

### 5-(6-Fluoropyridin-3-yl)-7-(2-oxoethoxy)imidazo[1,2-a]pyridine-3-carbonitrile (58a)

A mixture of 5-(6-fluoropyridin-3-yl)-7-hydroxyimidazo[1,2-a]pyridine-3-carbonitrile (1e) (50.0 mg, 0.196 mmol), 2-bromo-1,1-dimethoxyethane (132 mg, 0.786 mmol) and K₂CO₃ (35.0 mg, 0.978 mmol) in DMSO (2 mL) was stirred at 100° C. for overnight. The mixture was cooled to RT and diluted with EtOAc, washed with H₂O, brine, dried over Na₂SO₄ and concentrated. The residue was dissolved in DCM (2 mL) and then TFA (1 mL) was added. The mixture was stirred at RT for 4 h and concentrated to give the title compound 5-(6-fluoropyridin-3-yl)-7-(2-oxoethoxy)imidazo[1,2-a]pyridine-3-carbonitrile (58a). MS-ESI (m/z): 297 [M+1]⁺.

### 7-(2-((Dimethyl(oxo)-λ⁶-sulfanylidene)amino)ethoxy)-5-(6-fluoropyridin-3-yl)imidazo[1,2-a]pyridine-3-carbonitrile (58b)

To a solution of 5-(6-fluoropyridin-3-yl)-7-(2-oxoethoxy)imidazo[1,2-a]pyridine-3-carbonitrile (58a) (59.0 mg, 0.199 mmol) and iminodimethyl-λ⁶-sulfanone (60.0 mg, 0.638 mmol) in DCM (2 mL) was stirred at RT for 1 h. Then NaBH(OAc)₃ (212 mg, 1.00 mmol) was added, the mixture was stirred at RT for 0.5 h. The mixture was diluted with sat. NaHCO₃ aqueous solution and extracted with DCM/MeOH (10:1). The organic phase was washed with H₂O, dried over Na₂SO₄ and concentrated. The residue was purified by preparative TLC to give title compound 7-(2-((dimethyl(oxo)-λ⁶-sulfanylidene)amino)ethoxy)-5-(6-fluoropyridin-3-yl)imidazo[1,2-a]pyridine-3-carbonitrile (58b). MS-ESI (m/z): 374 [M+1]+.

### 7-(2-((Dimethyl(oxo)-λ⁶-sulfanylidene)amino)ethoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (58)

The title compound 7-(2-((dimethyl(oxo)-X⁶-sulfanylidene)amino)ethoxy)-5-(6-(6-((6-methoxypyridin-3-yl)methyl)-3,6-diazabicyclo[3.1.1]heptan-3-yl)pyridin-3-yl)imidazo[1,2-a]pyridine-3-carbonitrile (58) was prepared according to the synthetic method of 1 by replacing 5-(6-fluoropyridin-3-yl)-7-(1-methyl-1H-pyrazol-4-yl)imidazo[1,2-a]pyridine-3-carbonitrile (1g), tert-butyl piperazine-1-carboxylate and 4-fluorobenzaldehyde with 7-(2-((dimethyl(oxo)-λ⁶-sulfanylidene)amino)ethoxy)-5-(6-fluoropyridin-3-yl)imidazo[1,2-a]pyridine-3-carbonitrile (58b), tert-butyl 3,6-diazabicyclo[3.1.1]heptane-6-carboxylate and 6-methoxynicotinaldehyde. MS-ESI (m/z): 573 [M+1]+.

### Example 59

### 5-(6-(6-((6-Methoxypyridin-3-yl)methyl)-3,6-diazabicyclo[3.1.1]heptan-3-yl)pyridin-3-yl)-7-(4,4,4-trifluoro-3-hydroxybutoxy)imidazo[1,2-a]pyridine-3-carbonitrile (59)

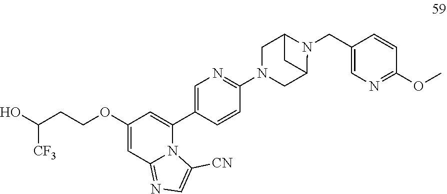 [SMILES: COc1ccc(CN2C3CC2CN(c2ccc(-c4cc(OCCC(O)C(F)(F)F)cc5ncc(C#N)n45)cn2)C3)cn1]

### 5-(6-Fluoropyridin-3-yl)-7-(4,4,4-trifluoro-3-hydroxybutoxy)imidazo[1,2-a]pyridine-3-carbonitrile (59a)

A mixture of 5-(6-fluoropyridin-3-yl)-7-hydroxyimidazo[1,2-a]pyridine-3-carbonitrile (1e) (25 mg, 0.098 mmol), 2-(trifluoromethyl)oxirane (112 mg, 1.00 mmol) and K₂CO₃ (41 mg, 0.297 mmol) in DMF (1 mL) was stirred at 80° C. in a sealed tube for 3 h. The mixture was cooled to rt and diluted with EtOAc, washed with H₂O, brine, dried over Na₂SO₄ and concentrated. The residue was purified by column chromatography on silica gel, eluting with DCM/MeOH (20:1) to give the title compound 5-(6-fluoropyridin-3-yl)-7-(4,4,4-trifluoro-3-hydroxybutoxy)imidazo[1,2-a]pyridine-3-carbonitrile (59a). 381 [M+1]+.

### 5-(6-(6-((6-Methoxypyridin-3-yl)methyl)-3,6-diazabicyclo[3.1.1]heptan-3-yl)pyri din-3-yl)-7-(4,4,4-trifluoro-3-hydroxybutoxy)imidazo[1,2-a]pyridine-3-carbonitrile (59)

The title compound 5-(6-(6-((6-methoxypyridin-3-yl)methyl)-3,6-diazabicyclo[3.1.1]heptan-3-yl)pyridin-3-yl)-7-(4,4,4-trifluoro-3-hydroxybutoxy)imidazo[1,2-a]pyridine-3-car bonitrile (59) was prepared according to the synthetic method of 1 by replacing 5-(6-fluoropyridin-3-yl)-7-(1-methyl-1H-pyrazol-4-yl)imidazo[1,2-a]pyridine-3-carbonitrile (1g), tert-butyl piperazine-1-carboxylate and 4-fluorobenzaldehyde with 5-(6-fluoropyridin-3-yl)-7-(4,4,4-trifluoro-3-hydroxybutoxy)imidazo[1,2-a]pyridine-3-carbonitrile (59a), tert-butyl 3,6-diazabicyclo[3.1.1]heptane-6-carboxylate and 6-methoxynicotinaldehyde. MS-ESI (m/z): 580 [M+1]⁺.

### Example 60

### 7-(2-Hydroxy-2-methylpropoxy)-5-(6-(4-((6-methoxypyridin-3-yl)oxy)piperidin-1-yl)pyridin-3-yl)imidazo[1,2-a]pyridine-3-carbonitrile (60)

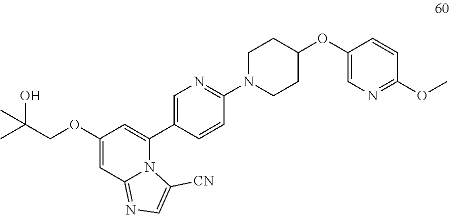 [SMILES: COc1ccc(OC2CCN(c3ccc(-c4cc(OCC(C)(C)O)cc5ncc(C#N)n45)cn3)CC2)cn1]

### Tert-butyl 4-((6-methoxypyridin-3-yl)oxy)piperidine-1-carboxylate (60a)

A mixture of tert-butyl 4-hydroxypiperidine-1-carboxylate (840 mg, 4.18 mmol), 6-methoxypyridin-3-ol (500 mg, 4.00 mmol), PPh₃ (1.36 g, 5.20 mmol) and DIAD (1.05 g, 5.20 mmol) in THF (10 mL) was stirred at 40° C. under N₂ atmosphere for 3 h. The mixture was cooled to RT and concentrated. The residue was purified by column chromatography on silica gel, eluting with PE/EtOAc (20:1) to give the title compound tert-butyl 4-((6-methoxypyridin-3-yl)oxy) piperidine-1-carboxylate (60a). MS-ESI (m/z): 309 [M+1]+.

### 2-methoxy-5-(piperidin-4-yloxy)pyridine (60b)

A mixture of tert-butyl 4-((6-methoxypyridin-3-yl)oxy) piperidine-1-carboxylate (60a) in HCl/EA (4 M, 1 mL) was stirred at RT for 1 h. The mixture was concentrated. The residue was diluted with H₂O, washed with PE/EtOAc (1:1). The aqueous layer was adjusted to pH=8. Extracted with DCM, washed with brine, dried over Na₂SO₄ and concentrated to give the title compound 2-methoxy-5-(piperidin-4-yloxy)pyridine (60b). MS-ESI (m/z): 209 [M+1]+.

### 7-(2-Hydroxy-2-methylpropoxy)-5-(6-(4-((6-methoxypyridin-3-yl)oxy)piperidin-1-yl)pyridin-3-yl)imidazo[1,2-a]pyridine-3-carbonitrile (60)

The title compound 7-(2-hydroxy-2-methylpropoxy)-5-(6-(4-((6-methoxypyridin-3-yl)oxy)piperidin-1-yl)pyridin-3-yl)imidazo[1,2-a]pyridine-3-carbonitrile (60) was prepared according to the synthetic method of 1h by replacing 5-(6-fluoropyridin-3-yl)-7-(1-methyl-1H-pyrazol-4-yl)imidazo[1,2-a]pyridine-3-carbonitrile (1g), tert-butyl piperazine-1-carboxylate with 5-(6-fluoropyridin-3-yl)-7-(2-hydroxy-2-methylpropoxy)imidazo[1,2-a]pyridine-3-carbonitrile (56a), and 2-methoxy-5-(piperidin-4-yloxy)pyridine (60b). MS-ESI (m/z): 515 [M+1]+.

### Example 61

### 7-(2-Hydroxy-2-methylpropoxy)-5-(6-(4-((6-(methoxy-d₃)pyridin-3-yl)oxy)piperidin-1-yl)pyridin-3-yl)imidazo[1,2-a]pyridine-3-carbonitrile (61)

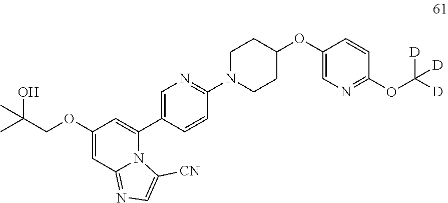 [SMILES: [2H]C([2H])([2H])Oc1ccc(OC2CCN(c3ccc(-c4cc(OCC(C)(C)O)cc5ncc(C#N)n45)cn3)CC2)cn1]

### 5-Bromo-2-(methoxy-d₃)pyridine 67a

A mixture of 2,5-dibromopyridine (237 mg, 1.00 mmol) and NaOH (40 mg, 1.00 mmol) in CD₃OD (1 mL) was stirred at 70° C. for overnight. The mixture was concentrated. The residue was dissolved in DCM, washed with brine, dried over Na₂SO₄ and concentrated. The residue was purified by column chromatography on silica gel, eluting with PE/EtOAc (10:1) to give the title compound 5-bromo-2-(methoxy-d₃)pyridine (61a). MS-ESI (m/z): 191/193 [M+1]+.

### (6-(Methoxy-d₃)pyridin-3-yl)boronic acid (61b)

To a solution of 5-bromo-2-(methoxy-d₃)pyridine (61a) (949 mg, 4.99 mmol) in THF (5 mL) was added n-BuLi (2.5 M in hexane, 3 mL, 7.50 mmol) at −78° C. The mixture was stirred at −78° C. for 5 min, triisopropyl borate (1.41 g, 7.5 mmol) was added dropwise. The mixture was warmed to RT and stirred for 1 h. The reaction was quenched with HCl (1N) and adjusted to pH=7 with NaOH (3N), extracted with EtOAc. The extracts were washed with brine, dried over Na₂SO₄ and concentrated to give the crude product of (6-(methoxy-d₃)pyridin-3-yl)boronic acid (61b), which was used for next step without further purification. MS-ESI (m/z): 157 [M+1]+.

### 6-(Methoxy-d₃)pyridin-3-ol (61c)

To a solution of (6-(methoxy-d₃)pyridin-3-yl)boronic acid (61b) (660 mg, 4.23 mmol) in THF (45 mL) was added NaOH aqueous solution (2 M, 10.6 mL) at 0° C. followed by H₂O₂ (30%, 3.8 mL, 33.8 mmol) and stirred for 2 h. The mixture was diluted with sat. Na₂S₂O₃ aqueous solution (20 mL), stirred at 0° C. for 0.5 h and washed with MTBE. The aqueous layer was adjusted to pH=2 with con. HCl, extracted with EtOAc, dried over Na₂SO₄ and concentrated. The residue was purified by column chromatography on silica gel, eluting with PE/EtOAc (5:1) to give the title compound 6-(methoxy-d₃)pyridin-3-ol (61c). MS-ESI (m/z): 129 [M+1]+.

### 7-(2-Hydroxy-2-methylpropoxy)-5-(6-(4-((6-(methoxy-d₃)pyridin-3-yl)oxy)piperidin-1-yl)pyridin-3-yl)imidazo[1,2-a]pyridine-3-carbonitrile (61)

The title compound 7-(2-hydroxy-2-methylpropoxy)-5-(6-(4-((6-(methoxy-d₃)pyridin-3-yl)oxy)piperidin-1-yl)pyridin-3-yl)imidazo[1,2-a]pyridine-3-carbonitrile (61) was prepared according to the synthetic method of 60 by replacing 6-methoxypyridin-3-ol with 6-(methoxy-d₃)pyridin-3-ol (61c). MS-ESI (m/z): 518 [M+1]+.

### Example 62

### 5-(5-Fluoro-6-(4-((6-methoxypyridin-3-yl)oxy)piperidin-1-yl)pyridin-3-yl)-7-(2-hydroxy-2-methylpropoxy)imidazo[1,2-a]pyridine-3-carbonitrile (62)

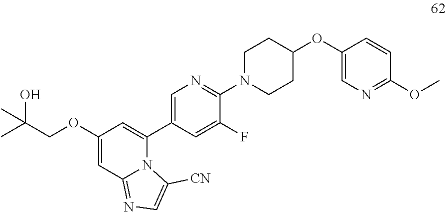 [SMILES: COc1ccc(OC2CCN(c3ncc(-c4cc(OCC(C)(C)O)cc5ncc(C#N)n45)cc3F)CC2)cn1]

### (5,6-Difluoropyridin-3-yl)boronic acid (62a)

A mixture of 5-bromo-2,3-difluoropyridine (600 mg, 3.09 mmol), bis(pinacolato)diboron (1.2 g, 4.72 mmol), Pd(dppf)Cl₂ (110 mg, 0.150 mmol) and KOAc (882 mg, 9.00 mmol) in dioxane (10 mL) was stirred at 100° C. under N2 atmosphere for overnight. The mixture was cooled to RT and filtered through celite. The filter cake was washed with EtOAc. The combined filtrate was concentrated to give the title compound (5,6-difluoropyridin-3-yl)boronic acid (62a). MS-ESI (m/z): 160 [M+1]+.

### (5-Fluoro-6-(4-((6-methoxypyridin-3-yl)oxy)piperidin-1-yl)pyridin-3-yl)boronic acid (62b)

A mixture of (5,6-difluoropyridin-3-yl)boronic acid (62a) (94 mg, 0.585 mmol), 2-methoxy-5-(piperidin-4-yloxy)pyridine (60b) (129 mg, 0.620 mmol) and K₂CO₃ (849 mg, 6.15 mmol) in dioxane (5 mL) was stirred at 80° C. for overnight. The mixture was cooled to RT and concentrated. The residue was purified by column chromatography on silica gel, eluting with DCM/MeOH (100:1) to give the title compound (5-fluoro-6-(4-((6-methoxypyridin-3-yl)oxy)piperidin-1-yl)pyridin-3-yl)boronic acid (62b). MS-ESI (m/z): 348 [M+1]+.

### 5-Bromo-7-hydroxyimidazo[1,2-a]pyridine-3-carbonitrile (62c)

The mixture of 5-bromo-7-methoxyimidazo[1,2-a]pyridine-3-carbonitrile (1c) (820 mg, 3.25 mmol) and AlCl₃ (2.17 g, 16.3 mmol) in DCE (10 mL) was stirred at 80° C. for 1.5 h. The mixture was cooled to RT, diluted with THF (20 mL), Na₂SO₄.10H₂O (10 g) was added, then stirred at RT for 0.5 h. The mixture was filtered through celite, washed with THF and the filtrate was concentrated to give the crude product of title compound 5-bromo-7-hydroxyimidazo[1,2-a]pyridine-3-carbonitrile (62c). MS-ESI (m/z): 238/240 [M+1]+.

### 5-(5-Fluoro-6-(4-((6-methoxypyridin-3-yl)oxy)piperidin-1-yl)pyridin-3-yl)-7-hydroxyimidazo[1,2-a]pyridine-3-carbonitrile (62d)

A mixture of 5-bromo-7-hydroxyimidazo[1,2-a]pyridine-3-carbonitrile (62c) (24.0 mg, 0.101 mmol), (5-fluoro-6-(4-((6-methoxypyridin-3-yl)oxy)piperidin-1-yl)pyridin-3-yl)boronic acid (62b) (52.0 mg, 0.149 mmol) and Pd(PPh₃)₄ (6.00 mg, 0.00519 mmol) in dioxane (1 mL) and Na₂CO₃ aqueous solution (2 M, 0.15 ml) was stirred at 90° C. under N₂ atmosphere for 6 h. The mixture was cooled to RT, diluted with EtOAc, washed with brine, dried over Na₂SO₄ and concentrated. The residue was purified by column chromatography on silica gel, eluting with DCM/MeOH (10:1) to give the title compound 5-(5-fluoro-6-(4-((6-methoxypyridin-3-yl)oxy)piperidin-1-yl)pyridin-3-yl)-7-hydroxyimidazo[1,2-a]pyridine-3-carbonitrile (62d). MS-ESI (m/z): 461 [M+1]+.

### 5-(5-Fluoro-6-(4-((6-methoxypyridin-3-yl)oxy)piperidin-1-yl)pyridin-3-yl)-7-(2-hydroxy-2-methylpropoxy)imidazo[1,2-a]pyridine-3-carbonitrile (62)

A mixture of 5-(5-fluoro-6-(4-((6-methoxypyridin-3-yl)oxy)piperidin-1-yl)pyridin-3-yl)-7-hydroxyimidazo[1,2-a]pyridine-3-carbonitrile (62d) (33.0 mg, 0.0717 mmol), 2,2-dimethyloxirane (52.0 mg, 0.722 mmol) and K₂CO₃ (30.0 mg, 0.219 mmol) in DMF (1 mL) was stirred at 80° C. in a sealed tube for overnight. The mixture was cooled to RT and diluted with H₂O, extracted with EtOAc. The organic phase was washed with H₂O, brine, dried over Na₂SO₄ and concentrated. The residue was purified by preparative TLC (DCM/MeOH=15:1) to give the title compound 5-(5-fluoro-6-(4-((6-methoxypyridin-3-yl)oxy)piperidin-1-yl)pyridin-3-yl)-7-(2-hydroxy-2-methylpropoxy)imidazo[1,2-a]pyridine-3-carbonitrile (62). MS-ESI (m/z): 533 [M+1]+.

### Example 63

### 5-(5-Fluoro-6-(4-((6-methoxypyridin-3-yl)oxy)piperidin-1-yl)pyridin-3-yl)-7-((1-hydroxycyclopropyl)methoxy)imidazo[1,2-a]pyridine-3-carbonitrile (63)

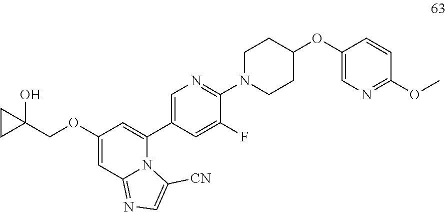 [SMILES: COc1ccc(OC2CCN(c3ncc(-c4cc(OCC5(O)CC5)cc5ncc(C#N)n45)cc3F)CC2)cn1]

### 5-Bromo-7-((1-((tert-butyldimethylsilyl)oxy)cyclopropyl)methoxy)imidazo[1,2-a]pyridine-3-carbonitrile (63a)

The title compound 5-bromo-7-((1-((tert-butyldimethylsilyl)oxy)cyclopropyl) methoxy)imidazo[1,2-a]pyridine-3-carbonitrile (63a) was prepared according to the synthetic method of 57c by replacing 5-(6-fluoropyridin-3-yl)-7-hydroxyimidazo[1,2-a]pyridine-3-carbonitrile (1e) with 5-bromo-7-hydroxyimidazo[1,2-a]pyridine-3-carbonitrile (62c). MS-ESI (m/z): 422/424 [M+1]+.

### 7-((1-((Tert-butyldimethylsilyl)oxy)cyclopropyl)methoxy)-5-(5-fluoro-6-(4-((6-methoxypyridin-3-yl)oxy)piperidin-1-yl)pyridin-3-yl)imidazo[1,2-a]pyridine-3-carbonitrile (63b)

The title compound 7-((1-((tert-butyldimethylsilyl)oxy)cyclopropyl)methoxy)-5-(5-fluoro-6-(4-((6-methoxypyridin-3-yl)oxy)piperidin-1-yl)pyridin-3-yl)imidazo[1,2-a]pyridine-3-carbonitrile (63b) was prepared according to the synthetic method of 62d by replacing 5-bromo-7-hydroxyimidazo[1,2-a]pyridine-3-carbonitrile (62c) with 5-bromo-7-((1-((tert-butyldimethylsilyl)oxy)cyclopropyl)methoxy)imidazo-[1,2-a]pyridine-3-carbonitrile (63a). MS-ESI (m/z): 645 [M+1]⁺.

### 5-(5-Fluoro-6-(4-((6-methoxypyridin-3-yl)oxy)piperidin-1-yl)pyridin-3-yl)-7-((1-hydroxycyclopropyl)methoxy)imidazo[1,2-a]pyridine-3-carbonitrile (63)

The title compound 5-(5-fluoro-6-(4-((6-methoxypyridin-3-yl)oxy)piperidin-1-yl)pyridin-3-yl)-7-((1-hydroxycyclopropyl)methoxy)imidazo[1,2-a]pyridine-3-carbonitrile (63) was prepared according to the synthetic method of 57d by replacing 7-((1-((tert-butyldimethylsilyl)oxy)cyclopropyl)methoxy)-5-(6-fluoropyridin-3-yl)imidazo[1,2-a]pyridine-3-carbonitrile (57c) with 7-((1-((tert-butyldimethylsilyl)oxy)cyclopropyl)methoxy)-5-(5-fluoro-6-(4-((6-methoxypyridin-3-yl)oxy)piperidin-1-yl)pyridin-3-yl)imidazo[1,2-a]pyridine-3-carbonitrile (63b). MS-ESI (m/z): 531 [M+1]⁺.

Following essentially the same procedures described for Examples 56-63, Examples 64-97 listed in Table 2 were prepared from the appropriate starting materials which are commercially available or known in the literature. The structures and names of Examples 64-97 are given in Table 2.

TABLE 1

| EXAMPLE | STRUCTURE | NAME | DATA |
|---|---|---|---|
| 64 | 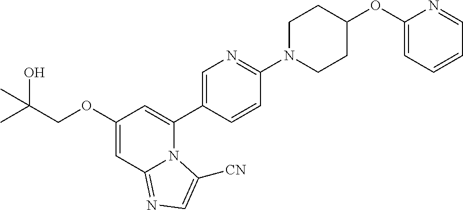 [SMILES: CC(C)(O)COc1cc(-c2ccc(N3CCC(Oc4ccccn4)CC3)nc2)n2c(C#N)cnc2c1] | 7-(2-hydroxy-2-methylpropoxy)-5-(6-(4- (pyridin-2-yloxy)piperidin-1-yl)pyridin-3- yl)imidazo[1,2-a]pyridine-3-carbonitrile | MS-ESI (m/z): 485 [M + 1]⁺ |
| 65 | 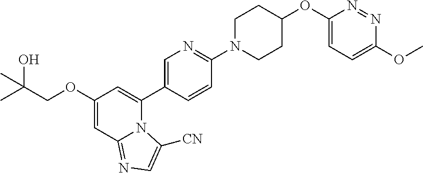 [SMILES: COc1ccc(OC2CCN(c3ccc(-c4cc(OCC(C)(C)O)cc5ncc(C#N)n45)cn3)CC2)nn1] | 7-(2-hydroxy-2-methylpropoxy)-5-(6-(4- ((6-methoxypyridazin-3-yl)oxy)piperidin- 1-yl)pyridin-3-yl)imidazo[1,2-a]pyridine- 3-carbonitrile | MS-ESI (m/z): 516 [M + 1]⁺ |
| 66 | 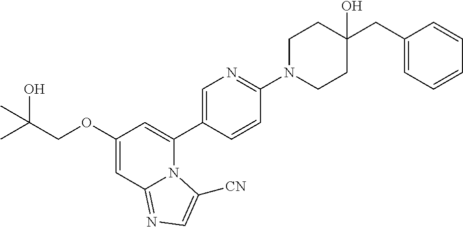 [SMILES: CC(C)(O)COc1cc(-c2ccc(N3CCC(O)(Cc4ccccc4)CC3)nc2)n2c(C#N)cnc2c1] | 5-(6-(4-benzyl-4-hydroxypiperidin-1-yl) pyridin-3-yl)-7-(2-hydroxy-2-methylpro- poxy)imidazo[1,2-a]pyridine-3-carbonitrile | MS-ESI (m/z): 498 [M + 1]⁺ |
| 67 | 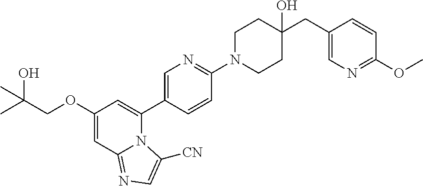 [SMILES: COc1ccc(CC2(O)CCN(c3ccc(-c4cc(OCC(C)(C)O)cc5ncc(C#N)n45)cn3)CC2)cn1] | 7-(2-hydroxy-2-methylpropoxy)-5-(6-(4- hydroxy-4-((6-methoxypyridin-3-yl)meth- yl)piperidin-1-yl)pyridin-3-yl)imidazo[1, 2-a]pyridine-3-carbonitrile | MS-ESI (m/z): 529 [M + 1]⁺ |
| 68 | 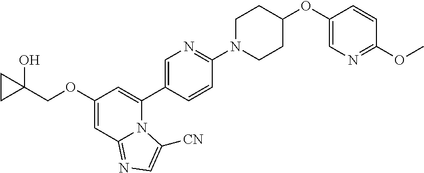 [SMILES: COc1ccc(OC2CCN(c3ccc(-c4cc(OCC5(O)CC5)cc5ncc(C#N)n45)cn3)CC2)cn1] | 7-((1-hydroxycyclopropyl)methoxy)-5-(6- (4-((6-methoxypyridin-3-yl)oxy)piperidin- 1-yl)pyridin-3-yl)imidazo[1,2-a]pyridine- 3-carbonitrile | MS-ESI (m/z): 513 [M + 1]⁺ |
| 69 | 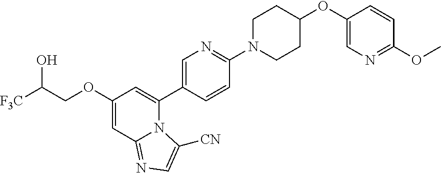 [SMILES: COc1ccc(OC2CCN(c3ccc(-c4cc(OCC(O)C(F)(F)F)cc5ncc(C#N)n45)cn3)CC2)cn1] | 5-(6-(4-((6-methoxypyridin-3-yl)oxy)pi- peridin-1-yl)pyridin-3-yl)-7-(3,3,3-trifluoro- 2-hydroxypropoxy)imidazo[1,2-a]pyri- dine-3-carbonitrile | MS-ESI (m/z): 555 [M + 1]⁺ |
| 70 | 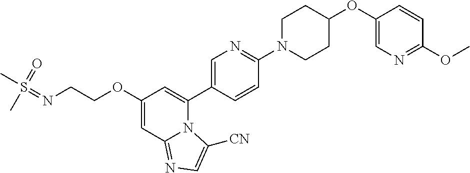 | 7-(2-((dimethyl(oxo)-λ⁶-sulfanylidene)ami- no)ethoxy)-5-(6-(4-((6-methoxypyridin- 3-yl)oxy)piperidin-1-yl)pyridin-3-yl)imid- azo[1,2-a]pyridine-3-carbonitrile | MS-ESI (m/z): 562 [M + 1]⁺ |
| 71 | 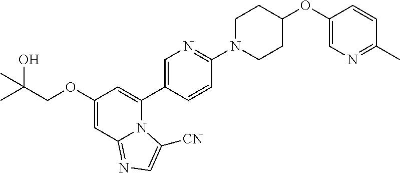 [SMILES: Cc1ccc(OC2CCN(c3ccc(-c4cc(OCC(C)(C)O)cc5ncc(C#N)n45)cn3)CC2)cn1] | 7-(2-hydroxy-2-methylpropoxy)-5-(6-(4- ((6-methylpyridin-3-yl)oxy)piperidin-1-yl) pyridin-3-yl)imidazo[1,2-a]pyridine-3- carbonitrile | MS-ESI (m/z): 499 [M + 1]⁺ |
| 72 | 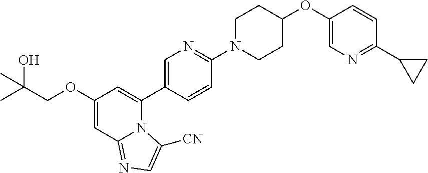 [SMILES: CC(C)(O)COc1cc(-c2ccc(N3CCC(Oc4ccc(C5CC5)nc4)CC3)nc2)n2c(C#N)cnc2c1] | 5-(6-(4-((6-cyclopropylpyridin-3-yl)oxy) piperidin-1-yl)pyridin-3-yl)-7-(2-hydroxy- 2-methylpropoxy)imidazo[1,2-a]pyridine- 3-carbonitrile | MS-ESI (m/z): 525 [M + 1]⁺ |
| 73 | 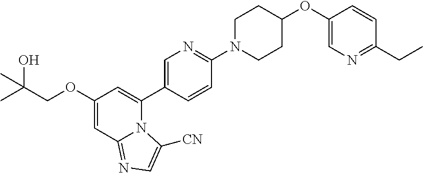 [SMILES: CCc1ccc(OC2CCN(c3ccc(-c4cc(OCC(C)(C)O)cc5ncc(C#N)n45)cn3)CC2)cn1] | 5-(6-(4-((6-ethylpyridin-3-yl)oxy)piperidin- 1-yl)pyridin-3-yl)-7-(2-hydroxy-2-meth- ylpropoxy)imidazo[1,2-a]pyridine-3-car- bonitrile | MS-ESI (m/z): 513 [M + 1]⁺ |
| 74 | 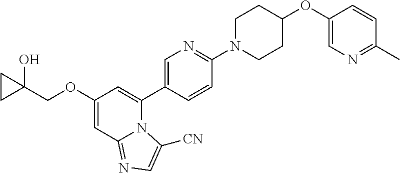 [SMILES: Cc1ccc(OC2CCN(c3ccc(-c4cc(OCC5(O)CC5)cc5ncc(C#N)n45)cn3)CC2)cn1] | 7-((1-hydroxycyclopropyl)methoxy)-5-(6- (4-((6-methylpyridin-3-yl)oxy)piperidin- 1-yl)pyridin-3-yl)imidazo[1,2-a]pyridine- 3-carbonitrile | MS-ESI (m/z): 497 [M + 1]⁺ |
| 75 | 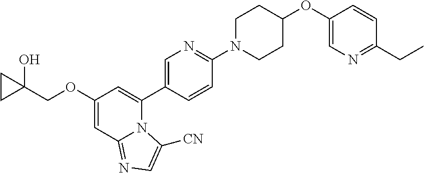 [SMILES: CCc1ccc(OC2CCN(c3ccc(-c4cc(OCC5(O)CC5)cc5ncc(C#N)n45)cn3)CC2)cn1] | 5-(6-(4-((6-ethylpyridin-3-yl)oxy)piperidin- 1-yl)pyridin-3-yl)-7-((1-hydroxycyclo- propyl)methoxy)imidazo[1,2-a]pyridine-3- carbonitrile | MS-ESI (m/z): 511 [M + 1]⁺ |
| 76 | 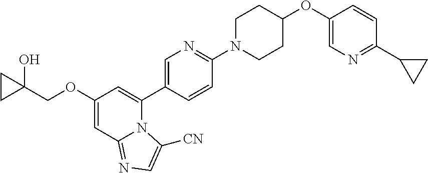 [SMILES: N#Cc1cnc2cc(OCC3(O)CC3)cc(-c3ccc(N4CCC(Oc5ccc(C6CC6)nc5)CC4)nc3)n12] | 5-(6-(4-((6-cyclopropylpyridin-3-yl)oxy) piperidin-1-yl)pyridin-3-yl)-7-((1-hydroxy- cyclopropyl)methoxy)imidazo[1,2-a]pyr- idine-3-carbonitrile | MS-ESI (m/z): 523 [M + 1]⁺ |
| 77 | 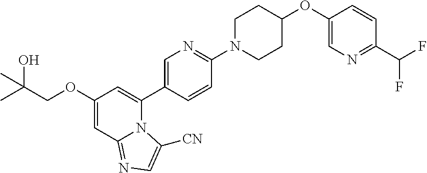 [SMILES: CC(C)(O)COc1cc(-c2ccc(N3CCC(Oc4ccc(C(F)F)nc4)CC3)nc2)n2c(C#N)cnc2c1] | 5-(6-(4-((6-(difluoromethyl)pyridin-3-yl) oxy)piperidin-1-yl)pyridin-3-yl)-7-(2-hy- droxy-2-methylpropoxy)imidazo[1,2-a]pyr- idine-3-carbonitrile | MS-ESI (m/z): 535 [M + 1]⁺ |
| 78 | 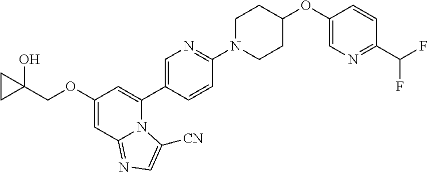 [SMILES: N#Cc1cnc2cc(OCC3(O)CC3)cc(-c3ccc(N4CCC(Oc5ccc(C(F)F)nc5)CC4)nc3)n12] | 5-(6-(4-((6-(difluoromethyl)pyridin-3-yl) oxy)piperidin-1-yl)pyridin-3-yl)-7-((1-hy- droxycyclopropyl)methoxy)imidazo[1,2- a]pyridine-3-carbonitrile | MS-ESI (m/z): 533 [M + 1]⁺ |
| 79 | 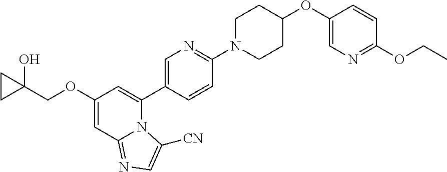 [SMILES: CCOc1ccc(OC2CCN(c3ccc(-c4cc(OCC5(O)CC5)cc5ncc(C#N)n45)cn3)CC2)cn1] | 5-(6-(4-((6-ethoxypyridin-3-yl)oxy)piperi- din-1-yl)pyridin-3-yl)-7-((1-hydroxycyclo- propyl)methoxy)imidazo[1,2-a]pyridine- 3-carbonitrile | MS-ESI (m/z): 527 [M + 1]⁺ |
| 80 | 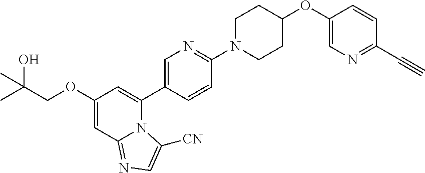 [SMILES: C#Cc1ccc(OC2CCN(c3ccc(-c4cc(OCC(C)(C)O)cc5ncc(C#N)n45)cn3)CC2)cn1] | 5-(6-(4-((6-ethynylpyridin-3-yl)oxy)piper- idin-1-yl)pyridin-3-yl)-7-(2-hydroxy-2- methylpropoxy)imidazo[1,2-a]pyridine-3- carbonitrile | MS-ESI (m/z): 509 [M + 1]⁺ |
| 81 | 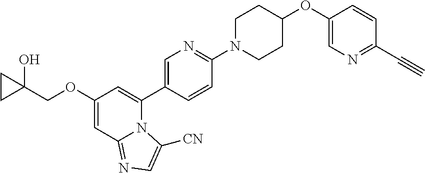 [SMILES: C#Cc1ccc(OC2CCN(c3ccc(-c4cc(OCC5(O)CC5)cc5ncc(C#N)n45)cn3)CC2)cn1] | 5-(6-(4-((6-ethynylpyridin-3-yl)oxy)piper- idin-1-yl)pyridin-3-yl)-7-((1-hydroxycyclo- propyl)methoxy)imidazo-[1,2-a]pyridine- 3-carbonitrile | MS-ESI (m/z): 507 [M + 1]⁺ |
| 82 | 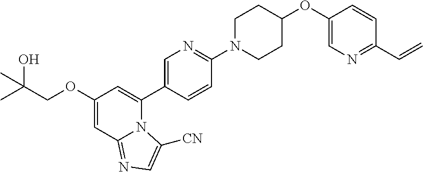 [SMILES: C=Cc1ccc(OC2CCN(c3ccc(-c4cc(OCC(C)(C)O)cc5ncc(C#N)n45)cn3)CC2)cn1] | 7-(2-hydroxy-2-methylpropoxy)-5-(6-(4- ((6-vinylpyridin-3-yl)oxy)piperidin-1-yl) pyridin-3-yl)imidazo[1,2-a]pyridine-3-car- bonitrile | MS-ESI (m/z): 511 [M + 1]⁺ |
| 83 | 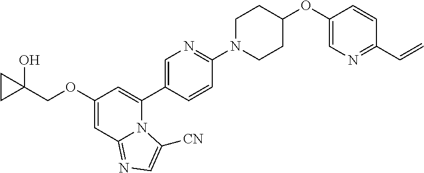 [SMILES: C=Cc1ccc(OC2CCN(c3ccc(-c4cc(OCC5(O)CC5)cc5ncc(C#N)n45)cn3)CC2)cn1] | 7-((1-hydroxycyclopropyl)methoxy)-5-(6- (4-((6-vinylpyridin-3-yl)oxy)piperidin-1- yl)pyridin-3-yl)imidazo[1,2-a]pyridine-3- carbonitrile | MS-ESI (m/z): 509 [M + 1]⁺ |
| 84 | 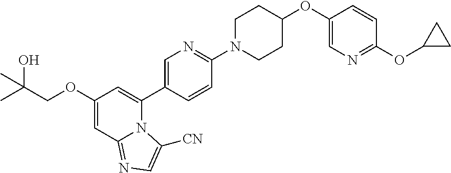 [SMILES: CC(C)(O)COc1cc(-c2ccc(N3CCC(Oc4ccc(OC5CC5)nc4)CC3)nc2)n2c(C#N)cnc2c1] | 5-(6-(4-((6-cyclopropoxypyridin-3-yl)oxy) piperidin-1-yl)pyridin-3-yl)-7-(2-hydroxy- 2-methylpropoxy)imidazo[1,2-a]pyri- dine-3-carbonitrile | MS-ESI (m/z): 541 [M + 1]⁺ |
| 85 | 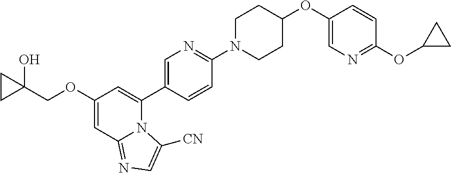 [SMILES: N#Cc1cnc2cc(OCC3(O)CC3)cc(-c3ccc(N4CCC(Oc5ccc(OC6CC6)nc5)CC4)nc3)n12] | 5-(6-(4-((6-cyclopropoxypyridin-3-yl)oxy) piperidin-1-yl)pyridin-3-yl)-7-((1-hy- droxycyclopropyl)methoxy)imidazo[1,2-a] pyridine-3-carbonitrile | MS-ESI (m/z): 539 [M + 1]⁺ |
| 86 | 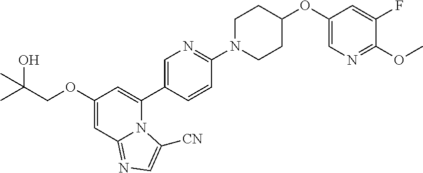 [SMILES: COc1ncc(OC2CCN(c3ccc(-c4cc(OCC(C)(C)O)cc5ncc(C#N)n45)cn3)CC2)cc1F] | 5-(6-(4-((5-fluoro-6-methoxypyridin-3-yl) oxy)piperidin-1-yl)pyridin-3-yl)-7-(2-hy- droxy-2-methylpropoxy)imidazo[1,2-a] pyridine-3-carbonitrile | MS-ESI (m/z): 533 [M + 1]⁺ |
| 87 | 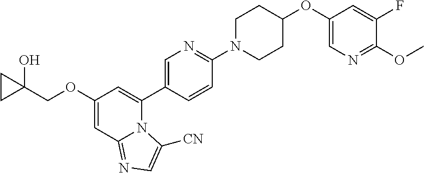 [SMILES: COc1ncc(OC2CCN(c3ccc(-c4cc(OCC5(O)CC5)cc5ncc(C#N)n45)cn3)CC2)cc1F] | 5-(6-(4-((5-fluoro-6-methoxypyridin-3-yl) oxy)piperidin-1-yl)pyridin-3-yl)-7-((1- hydroxycyclopropyl)methoxy)imidazo[1,2- a]pyridine-3-carbonitrile | MS-ESI (m/z): 531 [M + 1]⁺ |
| 88 | 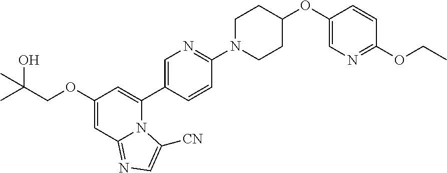 [SMILES: CCOc1ccc(OC2CCN(c3ccc(-c4cc(OCC(C)(C)O)cc5ncc(C#N)n45)cn3)CC2)cn1] | 5-(6-(4-((6-ethoxypyridin-3-yl)oxy)piperi- din-1-yl)pyridin-3-yl)-7-(2-hydroxy-2-meth- ylpropoxy)imidazo[1,2-a]pyridine-3-car- bonitrile | MS-ESI (m/z): 529 [M + 1]⁺ |
| 89 | 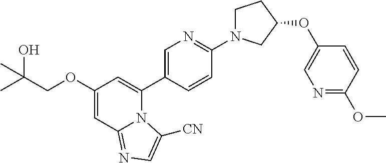 [SMILES: COc1ccc(O[C@H]2CCN(c3ccc(-c4cc(OCC(C)(C)O)cc5ncc(C#N)n45)cn3)C2)cn1] | (S)-7-(2-hydroxy-2-methylpropoxy)-5-(6- (3-((6-methoxypyridin-3-yl)oxy)pyrrolidin- 1-yl)pyridin-3-yl)imidazo[1,2-a]pyridine- 3-carbonitrile | MS-ESI (m/z): 501 [M + 1]⁺ |
| 90 | 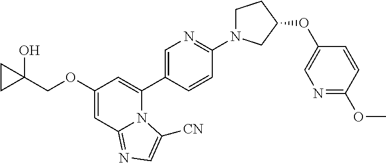 [SMILES: COc1ccc(O[C@H]2CCN(c3ccc(-c4cc(OCC5(O)CC5)cc5ncc(C#N)n45)cn3)C2)cn1] | (S)-7-((1-hydroxycyclopropyl)methoxy)- 5-(6-(3-((6-methoxypyridin-3-yl)oxy)pyr- rolidin-1-yl)pyridin-3-yl)imidazo[1,2-a] pyridine-3-carbonitrile | MS-ESI (m/z): 499 [M + 1]⁺ |
| 91 | 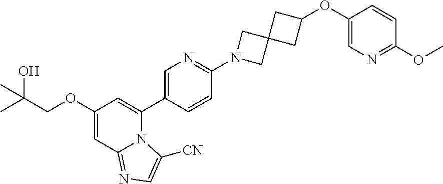 [SMILES: COc1ccc(OC2CC3(C2)CN(c2ccc(-c4cc(OCC(C)(C)O)cc5ncc(C#N)n45)cn2)C3)cn1] | 7-(2-hydroxy-2-methylpropoxy)-5-(6-(6- ((6-methoxypyridin-3-yl)oxy)-2-azaspiro [3.3]heptan-2-yl)pyridin-3-yl)imidazo[1,2- a]pyridine-3-carbonitrile | MS-ESI (m/z): 527 [M + 1]⁺ |
| 92 | 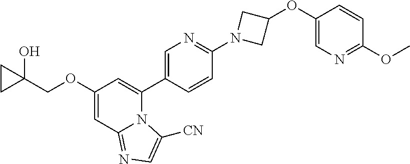 [SMILES: COc1ccc(OC2CN(c3ccc(-c4cc(OCC5(O)CC5)cc5ncc(C#N)n45)cn3)C2)cn1] | 7-((1-hydroxycyclopropyl)methoxy)-5-(6- (3-((6-methoxypyridin-3-yl)oxy)azetidin- 1-yl)pyridin-3-yl)imidazo[1,2-a]pyridine- 3-carbonitrile | MS-ESI (m/z): 485 [M + 1]⁺ |
| 93 | 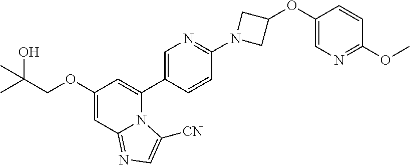 [SMILES: COc1ccc(OC2CN(c3ccc(-c4cc(OCC(C)(C)O)cc5ncc(C#N)n45)cn3)C2)cn1] | 7-(2-hydroxy-2-methylpropoxy)-5-(6-(3- ((6-methoxypyridin-3-yl)oxy)azetidin-1-yl) pyridin-3-yl)imidazo[1,2-a]pyridine-3- carbonitrile | MS-ESI (m/z): 487 [M + 1]⁺ |
| 94 | 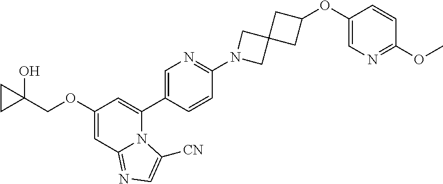 [SMILES: COc1ccc(OC2CC3(C2)CN(c2ccc(-c4cc(OCC5(O)CC5)cc5ncc(C#N)n45)cn2)C3)cn1] | 7-((1-hydroxycyclopropyl)methoxy)-5-(6- (6-((6-methoxypyridin-3-yl)oxy)-2-aza- spiro[3.3]heptan-2-yl)pyridin-3-yl)imidazo [1,2-a]pyridine-3-carbonitrile | MS-ESI (m/z): 525 [M + 1]⁺ |
| 95 | 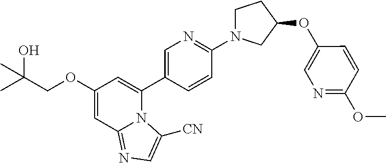 [SMILES: COc1ccc(O[C@@H]2CCN(c3ccc(-c4cc(OCC(C)(C)O)cc5ncc(C#N)n45)cn3)C2)cn1] | (R)-7-(2-hydroxy-2-methylpropoxy)-5-(6- (3-((6-methoxypyridin-3-yl)oxy)pyrrolidin- 1-yl)pyridin-3-yl)imidazo[1,2-a]pyridine- 3-carbonitrile | MS-ESI (m/z): 501 [M + 1]⁺ |
| 96 | 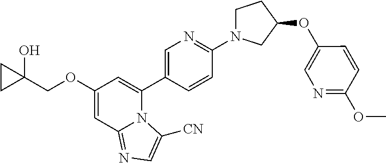 [SMILES: COc1ccc(O[C@@H]2CCN(c3ccc(-c4cc(OCC5(O)CC5)cc5ncc(C#N)n45)cn3)C2)cn1] | (R)-7-((1-hydroxycyclopropyl)methoxy)- 5-(6-(3-((6-methoxypyridin-3-yl)oxy)pyr- rolidin-1-yl)pyridin-3-yl)imidazo[1,2-a] pyridine-3-carbonitrile | MS-ESI (m/z): 499 [M + 1]⁺ |
| 97 | 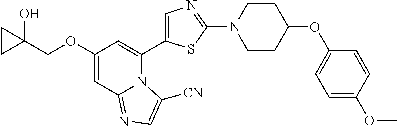 [SMILES: COc1ccc(OC2CCN(c3ncc(-c4cc(OCC5(O)CC5)cc5ncc(C#N)n45)s3)CC2)cc1] | 7-((1-hydroxycyclopropyl)methoxy)-5-(2- (4-(4-methoxyphenoxy)piperidin-1-yl) thiazol-5-yl)imidazo[1,2-a]pyridine-3-car- bonitrile | MS-ESI (m/z): 518 [M + 1]⁺ |

Cell Proliferation Assays

MTS testing kit was purchased from Promega. The RPMI-1640, F12, F12K, Fetal bovine serum and Penicillin-Streptomycin were purchased from BI. Glutamine and Dimethyl sulfoxide (DMSO) were purchased from Sigma. TT cells were cultured in F12K supplemented with 10% FBS and LC-2/ad cells were cultured in HamF12: RPMI1640 (1:1) supplemented with 10% FBS and 2 mM Glutamine.

To investigate whether a compound is able to inhibit the activity of RET fusion and/or mutation in cells, a mechanism-based assay using TT (RET C634W) and LC-2/ad (CCDCl₆-RET) cell lines was developed. In this assay, the inhibition of RET fusion and/or mutation was reflected by the inhibition of cell proliferation of TT and LC-2/ad cells. Cells were plated into 96-well plates at the optimized cell density (TT: 5000 cells/well; LC-2/ad: 5000 cells/well). Plates were incubated at 37° C., with 5% CO₂ for 24 h. Compounds were serially diluted and added to the plates with the final concentrations as 10000, 3333.3, 1111.1, 270.4, 123.5, 41.2, 13.7, 4.6 and 1.5 nM. Plates were incubated at 37° C., with 5% CO₂ for 72 h. An aliquot of 20 μL MTS/100 μL medium mixture solution were added to each well and the plates were incubated for exactly 2 h. The reaction was stopped by adding 25 μL 10% SDS to each well. The absorbance was measured by a microplate reader at 490 nm and 650 nm (reference wavelength). IC₅₀ was calculated using GraphPad Prism 5.0.

Selected compounds prepared as described above were assayed according to the biological procedures described herein. The results are shown in the table 2.

TABLE 2

| Example | TT IC₅₀ (nM) | LC-2/ad IC₅₀ (nM) |
|---|---|---|
| 2 | 63 | 54 |
| 5 | 71 | 80 |
| 6 | 42 | 44 |
| 7 | 11 | 16 |
| 8 | 39 | / |
| 9 | 79 | / |
| 10 | 6 | / |
| 14 | 6 | 13 |
| 17 | 48 | 45 |
| 18 | 1 | 2 |
| 19 | 1 | 6 |
| 20 | 6 | 17 |
| 21 | 12 | 12 |
| 22 | 4 | 1 |
| 24 | 86 | / |
| 25 | 79 | / |
| 26 | 74 | 89 |
| 27 | 65 | 39 |
| 28 | 79 | / |
| 29 | 84 | / |
| 33 | 34 | 17 |
| 35 | 1 | / |
| 36 | 62 | / |
| 37 | 45 | 41 |
| 38 | 38 | 34 |
| 39 | 39 | / |
| 41 | 12 | / |
| 45 | 66 | 50 |
| 46 | 37 | 36 |
| 47 | 88 | / |
| 48 | 77 | 50 |
| 50 | 14 | 30 |
| 51 | 44 | 40 |
| 52 | 24 | 42 |
| 57A | 33 | 81 |
| 57B | 127 | 115 |
| 58 | 120 | / |
| 59 | 126 | 64 |
| 60 | 3 | 9 |
| 61 | 15 | / |
| 64 | 87 | 28 |
| 65 | 38 | 34 |
| 66 | 117 | 55 |
| 68 | 5 | 10 |
| 69 | 57 | 49 |
| 70 | 71 | / |
| 71 | 22 | / |
| 72 | 17 | / |
| 73 | 1 | / |
| 74 | 23 | / |
| 75 | 1 | / |
| 76 | 5 | / |
| 77 | 48 | / |
| 78 | 13 | / |
| 79 | 1 | / |
| 80 | 7 | / |
| 81 | 5 | / |
| 82 | 15 | / |
| 83 | 17 | / |
| 84 | 21 | / |
| 85 | 12 | / |
| 87 | 38 | / |
| 88 | 2 | / |
| 89 | 45 | / |
| 90 | 13 | / |
| 92 | 87 | / |
| 93 | 39 | / |
| 95 | 7 | / |
| 96 | 9 | / |
| / | / | / |

1. A compound of formula (I): 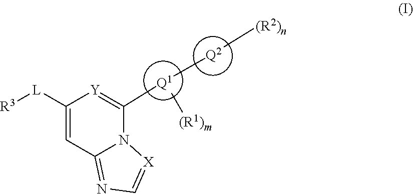
or a pharmaceutically acceptable salt thereof, wherein:
Q¹ is selected from aryl and heteroaryl;
Q² is heterocyclyl;
X is selected from CR⁴ and N;
Y is selected from CR⁵ and N;
L is selected from a bond, —(CR^{C0}R^{D0})ᵤ—, —(CR^{C0}R^{D0})ᵤO(CR^{C0}R^{D0})ₜ—(CR^{C0}R^{D0})ᵤNR^{A0}(CR^{C0}R^{D0})ₜ—, —(CR^{C0}R^{D0})ᵤS(CR^{C0}R^{D0})ₜ—, —(CR^{C0}R^{D0})ᵤC(O)NR^{A0}(CR^{C0}R^{D0})ₜ—, —(CR^{C0}R^{D0})NR^{A0}C(O)(CR^{C0}R^{D0})ₜ—, —(CR^{C0}R^{D0})NR^{A0}C(O)NR^{B0}(CR^{C0}R^{D0})ₜ—, —(CR^{C0}R^{D0})ᵤS(O)ᵣ(CR^{C0}R^{D0})ₜ—, —(CR^{C0}R^{D0})ᵤS(O)ᵣNR^{A0}(CR^{C0}R^{D0})ₜ—, —(CR^{C0}R^{D0})ᵤNR^{A0}S(O)ᵣ(CR^{C0}R^{D0})ₜ—, and —(CR^{C0}R^{D0})ᵤNR^{A0}S(O)ᵣNR^{B0}(CR^{C0}R^{D0})ₜ;
each R¹ is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A1}R^{B1}, —OR^{A1}, —C(O)R^{A1}, —C(═ NR^{E1})R^{A1}, —C(═N—OR^{B1})R^{A1}, —C(O)OR^{A1}, —OC(O)R^{A1}, —C(O)NR^{A1}R^{B1}, —NR^{A1}C(O)R^{B1}, —C(═NR^{E1})R^{A1}R^{B1},—NR^{A1}C(═N^{E1})R^{B1}, —OC(O)NR^{A1}R^{B1}, —NR^{A1}C(O)OR^{B1}, —NR^{A1}C(O)NR^{A1}R^{B1}, —NR^{A1}C(S)NR^{A1}R^{B1}, —NR^{A1}C(═NR^{E1})NR^{A1}R^{B1}, —S(O)ᵣR^{A1}, —S(O)(═NR^{E1})R^{B1}, —N═S(O)R^{A1}R^{B1}, —S(O)₂OR^{A1}, —OS(O)₂R^{A1}, —NR^{A1}S(O)ᵣR^{B1}, —NR^{A1}S(O)(═NR^{E1})R^{B1}, —S(O)ᵣNR^{A1}R^{B1}, —S(O)(═NR^{E1})NR^{A1}R^{B1}, —NR^{A1}S(O)₂NR^{A1}R^{B1}, —NR^{A1}S(O)(═NR^{E1}NR^{A1}R^{B1}, —P(O)R^{A1}R^{B1} and —P(O)(OR^{A1})(OR^{B}1), wherein alkyl, alkenyl, alkynyl, cycloalkyl, heterocyclyl, aryl and heteroaryl are each unsubstituted or substituted with at least one substituent, independently selected from R^{X};
each R² is independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A2}R^{B2}, —OR^{A2}, —C(O)R^{A2}, —C(═NR^{E2})R^{A2}, —C(═N—OR^{B2})R^{A2}, —C(O)OR^{A2}, —OC(O)R^{A2}, —C(O)NR^{A2}R^{B2}, —NR^{A2}C(O)R^{B2}, —C(═NR^{E2})NR^{A2}R^{B2}, —NR^{A2}C(═NR^{E2})R^{B2}, —OC(O)NR^{A2}R^{B2}, —NR^{A2}C(O)OR^{B2}, —NR^{A2}C(O)NR^{A2}R^{B2}, —NR^{A2}C(S)NR^{A2}R^{B2}, —NR^{A2}C(═NR^{E2})NR^{A2}R^{B2}, —S(O)ᵣR^{A2}, —S(O)(═NR^{E2})R^{B2}, —N═S(O)R^{A2}R^{B2}, —S(O)₂OR^{A2}, —OS(O)₂R^{A2}, —NR^{A2}S(O)ᵣR^{B2}, —NR^{A2}S(O)(═NR^{E2})R^{B2}, —S(O)ᵣNR^{A2}R^{B2}, —S(O)(═NR^{E2})NR^{A2}R^{B2}, —NR^{A2}S(O)₂NR^{A2}R^{B2}, —NR^{A2}S(O)(═NR^{E2})NR^{A2}R^{B2}, —P(O)R^{A2}R^{B2} and —P(O)(OR^{A2})(OR^{B2}), wherein alkyl, alkenyl, alkynyl, cycloalkyl, heterocyclyl, aryl and heteroaryl are each unsubstituted or substituted with at least one substituent, independently selected from R^{X};
R³ is selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, CN, NO₂, —NR^{A3}R^{B3}, —OR^{A3}, —C(O)R^{A3}, —C(═NR^{E3})R^{A3}, —C(═N—OR^{B3})R^{A3}, —C(O)OR^{A3}, —OC(O)R^{A3}, —C(O)NR^{A3}R^{B3}, —NR^{A3}C(O)R^{B3}, —C(═NR^{E3})NR^{A3}R^{B3}, —NR^{A3}C(═NR^{E3})R^{B3}, —OC(O)NR^{A3}R^{B3}, —NR^{A3}C(O)OR^{B3}, —NR^{A3}C(O)NR^{A3}R^{B3}, —NR^{A3}C(S)NR^{A3}R^{B3}, —NR^{A3}C(═NR^{E3})NR^{A3}R^{B3}, —S(O)ᵣR^{A3}, —S(O)(═NR^{E3})R^{B3}, —N═S(O)R^{A3}R^{B3}, —S(O)₂OR^{A3}, —OS(O)₂R^{A3}, —NR^{A3}S(O)ᵣR^{B3}, —NR^{A3}S(O)(═NR^{E3})R^{B3}, —S(O)ᵣNR^{A3}R^{B3}, —S(O)(═NR^{E3})NR^{A3}R^{B3}, —NR^{A3}S(O)₂NR^{A3}R^{B3}, —NR^{A3}S(O)(═NR^{E3})NR^{A3}R^{B3}, —P(O)R^{A3}R^{B3} and —P(O)(OR^{A3})(OR^{B3}), wherein alkyl, alkenyl, alkynyl, cycloalkyl, heterocyclyl, aryl and heteroaryl are each unsubstituted or substituted with at least one substituent, independently selected from R⁶;
R⁴ and R⁵ are independently selected from hydrogen, halogen, CN, C₁₋₁₀ alkyl and C₃₋₁₀ cycloalkyl, wherein alkyl and cycloalkyl are unsubstituted or substituted with at least one substituent, independently selected from R^{X};
R⁶ is selected from hydrogen, halogen, OH, C₁₋₁₀ alkyl, C₃₋₁₀ cycloalkyl, C₃₋₁₀ cycloalkyl-C₁₋₄ alkyl, heterocyclyl and heterocyclyl-C₁₋₄ alkyl, wherein alkyl, cycloalkyl and heterocyclyl are unsubstituted or substituted with at least one substituent, independently selected from Rx;
each R^{A0}, R^{A1}, R^{A2}, R^{A3}, R^{B0}, R^{B1}, R^{B2} and R^{B3} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from Rx;
or each “R^{A0} and R^{B0}”, “R^{A1} and R^{B1}”, “R^{A2} and R^{B2}” or “R^{A3} and R^{B3}” together with the atom(s) to which they are attached form a heterocyclic ring of 4 to 12 members containing 0, 1 or 2 additional heteroatoms independently selected from oxygen, sulfur, nitrogen and phosphorus and optionally substituted with 1, 2 or 3 Rx groups;
each R^{C0} and R^{D0} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from Rx;
or R^{C0} and R^{D0} together with the carbon atom(s) to which they are attached form a ring of 3 to 12 members containing 0, 1 or 2 heteroatoms independently selected from oxygen, sulfur and nitrogen and optionally substituted with 1 2 or 3 Rx groups;
each R^{E1}, R^{E2} and R^{E3} are independently selected from hydrogen, C₁₋₁₀ alkyl, CN, NO₂, —OR^{a1}, —SR^{a1}, —S(O)ᵣR^{a1}, —C(O)R^{a1}, —C(O)OR^{a1}, —C(O)NR^{a1}R^{b1} and —S(O)ᵣNR^{a1}R^{b1}, wherein alkyl is unsubstituted or substituted with at least one substituent, independently selected from Rx;
each Rx is independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CRl^{c1}R^{d1})ₜNR^{a1}R^{b1}, —(CR^{c1}R^{d1})ₜOR^{b1}, —(CR^{c1}R^{d1})ₜC(O)R^{a1}, —(CR^{c1}R^{d1})ₜC(═NR^{e1})R^{a1}, —(CR^{c1}R^{d1})ₜC(═N—OR^{b1})R^{a1}, —(CR^{c1}R^{d1})ₜC(O)OR^{b1}, —(CR^{c1}R^{d1})ₜOC(O)R^{b1}, —(CR^{c1}R^{d1})ₜC(O)NR^{a1}R^{b1}, —(CR^{c1}R^{d1})NR^{a1}C(O)R^{b1}, —(CR^{c1}R^{d1})ₜC(═NR^{e1})NR^{a1}R^{b1}, —(CR^{c1}R^{d1})ₜNR^{a1}C(═NR^{e1})R^{b1}, —(CR^{c1}R^{d1})ₜOC(O)NR^{a1}R^{b1}, —(CR^{c1}R^{d1})NR^{a1}C(O)O^{Rb1}, —(CR^{c1}R^{d1})NR^{a1}C(O)NR^{a1}R^{b1}, —(CR^{c1}R^{d1})ₜNR^{a1}C(S)NR^{a1}R^{b1}, —(CR^{c1}R^{d1})ₜR^{a1}C(═NR^{e1})NR^{a1}R^{b1}, —(CR^{c1}R^{d1})ₜS(O)ᵣR^{b1}, —(CR^{c1}R^{d1})ₜS(O)(═NR^{e1})R^{b1}, —(CR^{c1}R^{d1})ₜN═S(O)R^{a1}R^{b1}, —(CR^{c1}R^{d1})ₜS(O)₂OR^{b1}, —(CR^{c1}R^{d1})ₜOS(O)₂R^{b1}, —(CR^{c1}R^{d1})ₜR^{a1}S(O)ᵣR^{b1}, —(CR^{c1}R^{d1})NR^{a1}S(O)(═NR^{e1})R^{b1}, —(CR^{c1}R^{d1})ₜS(O)ᵣNR^{a1}R^{b1}, —(CR^{c1}R^{d1})ₜS(O)(═NR^{e1})NR^{a1}R^{b1}, —(CR^{c1}R^{d1})ₜNR^{a1}S(O)₂NR^{a1}R^{b1}, —(CR^{c1}R^{d1})NR^{a1}S(O)(═NR^{e1})NR^{a1}R^{b1}, —(CR^{c1}R^{d1})ₜP(O)R^{a1}R^{b1} and —(CR^{c1}R^{d1})ₜP(O)(OR^{a1})(OR^{b1}) wherein alkyl, alkenyl, alkynyl, cycloalkyl, heterocyclyl, aryl and heteroaryl are each unsubstituted or substituted with at least one substituent, independently selected from R^{Y};
each R^{a1} and each R^{b1} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};
or R^{a1} and R^{b1} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1, 2 or 3 R^{Y} groups;
each R^{c1} and each R^{d1} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, heterocyclyl, aryl and heteroaryl are each unsubstituted or substituted with at least one substituent, independently selected from R^{Y};
or R^{c1} and R^{d1} together with the carbon atom(s) to which they are attached form a ring of 3 to 12 members containing 0, 1 or 2 heteroatoms independently selected from oxygen, sulfur and nitrogen, and optionally substituted with 1, 2 or 3 R^{Y} groups;
each Ret is independently selected from hydrogen, C₁₋₁₀ alkyl, C₃₋₁₀ cycloalkyl, C₃₋₁₀ cycloalkyl-C₁₋₄ alkyl, CN, NO₂, —OR^{a2}, —SR^{a2}, —S(O)ᵣR^{a2}, —C(O)R^{a2}, —C(O)OR^{a2}, —S(O)ᵣNR²R^{b2} and —C(O)NR^{a2}R^{b2};
each R^{Y} is independently selected from C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, heterocyclyl, heterocyclyl-C₁₋₄ alkyl, aryl, aryl-C₁₋₄ alkyl, heteroaryl, heteroaryl-C₁₋₄ alkyl, halogen, CN, NO₂, —(CR^{c2}R^{d2})NR^{a2}R^{b2}, —(CR^{c2}R^{d2})ₜOR^{b2}, —(CR^{c2}R^{d2})ₜC(O)R^{a2}, —(CR^{c2}R^{d2})ₜC(═NR^{e2})R^{a1}, —(CR^{c2}R^{d2})ₜC(═N—OR^{b2})R^{a2}, —(CR^{c2}R^{d2})ₜC(O)OR^{b2}, —(CR^{c2}R^{d2})ₜOC(O)R^{b2}, —(CR^{c2}R^{d2})ₜC(O)NR^{a2}R^{b2}, —(CR^{c2}R^{d2})NR^{a2}C(O)R^{b2}, —(CR²R^{d2})ₜC(═NR^{e2})NR^{a2}R^{b2}, —(CR^{c2}R^{d2})ₜNR^{a2}C(═NR^{e2})R^{b2}, —(CR²R²)ₜOC(O)NR^{a2}R^{b2}, —(CR^{c2}R^{d2})ₜNR^{a2}C(O)OR^{b2}, —(CR^{c2}R^{d2})ₜNR^{a2}C(O)NR^{a2}R^{b2}, —(CR^{c2}R^{d2})ₜNR^{a2}C(S)NR^{a2}R^{b2}, —(CR^{c2}R^{d2})NR^{a2}C(═NR^{e2})NR^{a2}R^{b2}, (CR^{c2}R^{d2})ₜS(O)ᵣR^{b2}, —(CR^{c2}R^{d2})ₜS(O)(═NR^{e2})R^{b2}, —(CR^{c2}R^{d2})ₜN═S(O)R^{a2}R^{b2}, —(CR^{c2}R^{d2})ₜS(O)₂OR^{b2}, —(CR^{c2}R^{d2})ₜOS(O)₂R^{b2}, —(CR²R^{d2})ₜNR^{a2}S(O)ᵣR^{b2}, —(CR^{e2}R^{d2})ₜNR^{a2}S(O)(═NR^{e2})R^{b2}, —(CR^{c2}R^{d2})ₜS(O)ᵣNR^{a2}R^{b2}, —(CR^{c2}R^{a2})ₜS(O)(═NR^{e2})NR^{a2}R^{b2}, (CR^{c2}R^{d2})ₜNR^{a2}S(O)₂NR^{a2}R^{b2}, —(CR^{c2}R^{d2})ₜNR^{a2}S(O)(═NR^{e2})NR^{a2}R^{b2}, (CR^{c2}R^{d2})ₜP(O)R^{a2}R^{b2} and —(CR^{c2}R^{d2})ₜP(O)(OR^{a2})(OR^{b2}), wherein alkyl, alkenyl, alkynyl, cycloalkyl, heterocyclyl, aryl and heteroaryl are each unsubstituted or substituted with at least one substituent, independently selected from OH, CN, amino, halogen, C₁₋₁₀ alkyl, C₂₋₁₀ alkenyl, C₂₋₁₀ alkynyl, C₃₋₁₀ cycloalkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino and di(C₁₋₁₀ alkyl)amino;
each R^{a2} and each R^{b2} are independently selected from hydrogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R² and R^{b2} together with the atom(s) to which they are attached form a heterocyclic ring of 4 to 12 members containing 0, 1 or 2 additional heteroatoms independently selected from oxygen, sulfur, nitrogen and phosphorus, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{c2} and each R^{d2} are independently selected from hydrogen, halogen, C₁₋₁₀ alkyl, C₂₋₁₀ alkenyl, C₂₋₁₀ alkynyl, C₃₋₁₀ cycloalkyl, C₃₋₁₀ cycloalkyl-C₁₋₄ alkyl, C₁₋₁₀ alkoxy, C₃₋₁₀ cycloalkoxy, C₁₋₁₀ alkylthio, C₃₋₁₀ cycloalkylthio, C₁₋₁₀ alkylamino, C₃₋₁₀ cycloalkylamino, di(C₁₋₁₀ alkyl)amino, heterocyclyl, heterocyclyl-C₁₋₄ alkyl, aryl, aryl-C₁₋₄ alkyl, heteroaryl and heteroaryl-C₁₋₄ alkyl, wherein alkyl, alkenyl, alkynyl, cycloalkyl, alkoxy, cycloalkoxy, alkylthio, cycloalkylthio, alkylamino, cycloalkylamino, heterocyclyl, aryl and heteroaryl are each unsubstituted or substituted with at least one substituent, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
or R^{c2} and R^{d2} together with the carbon atom(s) to which they are attached form a ring of 3 to 12 members containing 0, 1 or 2 heteroatoms independently selected from oxygen, sulfur and nitrogen, and optionally substituted with 1 or 2 substituents, independently selected from halogen, CN, C₁₋₁₀ alkyl, C₂₋₁₀ alkenyl, C₂₋₁₀ alkynyl, C₃₋₁₀ cycloalkyl, OH, C₁₋₁₀ alkoxy, C₃₋₁₀ cycloalkoxy, C₁₋₁₀ alkylthio, C₃₋₁₀ cycloalkylthio, amino, C₁₋₁₀ alkylamino, C₃₋₁₀ cycloalkylamino and di(C₁₋₁₀ alkyl)amino;
each R^{e2} is independently selected from hydrogen, CN, NO₂, C₁₋₁₀ alkyl, C₃₋₁₀ cycloalkyl, C₃₋₁₀ cycloalkyl-C₁₋₄ alkyl, C₁₋₁₀ alkoxy, C₃₋₁₀ cycloalkoxy, —C(O)C₁₋₄ alkyl, —C(O)C₃₋₁₀ cycloalkyl, —C(O)OC₁₋₄ alkyl, —C(O)OC₃₋₁₀ cycloalkyl, —C(O)N(C₁₋₄ alkyl)₂, —C(O)N(C₃₋₁₀ cycloalkyl)₂, —S(O)₂C₁₋₄ alkyl, —S(O)₂C₃₋₁₀ cycloalkyl, —S(O)₂N(C₁₋₄ alkyl)₂ and —S(O)₂N(C₃₋₁₀ cycloalkyl)₂;
m is selected from 1, 2 and 3;
n is selected from 1, 2 and 3;
each r is independently selected from 0, 1 and 2;
each t is independently selected from 0, 1, 2, 3 and 4;
each u is independently selected from 0, 1, 2, 3 and 4.

2. The compound of claim 1 or a pharmaceutically acceptable salt thereof, wherein L is a bond, R³ is pyrazolyl, and the compound has the formula (II), 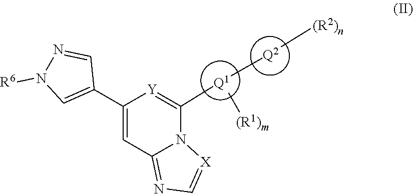
wherein Q¹, Q², X, Y, R¹, R², R⁶, n and m are as defined in formula (I); or
wherein L is O, and the compound has the formula (III), 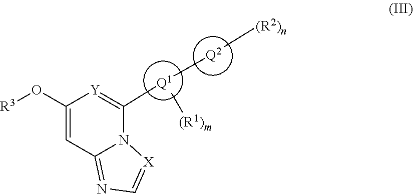
wherein Q¹, Q², X, Y, R¹, R², R³, n and m are as defined in formula (I);
wherein Y is CH, and the compound has the formula (IV), 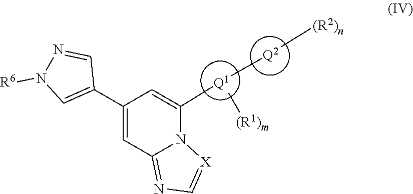
wherein Q¹, Q², X, R¹, R², R⁶, n and m are as defined in formula (I); or
wherein Y is N, and the compound has the formula (V), 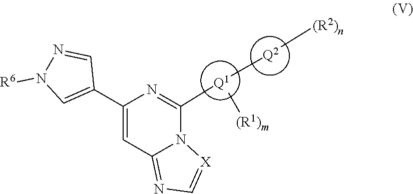
wherein Q¹, Q², X, R¹, R², R⁶, n and m are as defined in formula (I).

3. (canceled)

4. (canceled)

5. (canceled)

6. The compound of claim 2 or a pharmaceutically acceptable salt thereof, wherein R⁶ is C₁₋₁₀ alkyl, wherein alkyl is unsubstituted or substituted with at least one substituent independently selected from R^{X}.

7. The compound of claim 6 or a pharmaceutically acceptable salt thereof, wherein R⁶ is selected from methyl,

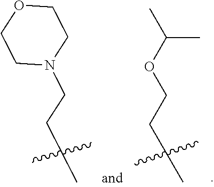

8. The compound of claim 3 or a pharmaceutically acceptable salt thereof, wherein R³ is selected from C₁₋₁₀ alkyl, wherein alkyl is unsubstituted or substituted with at least one substituent, independently selected from R⁶.

9. The compound of claim 8 or a pharmaceutically acceptable salt thereof, wherein R³ is selected from methyl and ethyl, and methyl and ethyl are each unsubstituted or substituted with at least one substituent, independently selected from R⁶, and R⁶ is selected from C₁₋₁₀ alkyl, C₃₋₁₀ cycloalkyl and OH, wherein alkyl and cycloalkyl is unsubstituted or substituted with at least one substituent, independently selected from Rx.

10. The compound of claim 9 or a pharmaceutically acceptable salt thereof, wherein R⁶ is selected from methyl, cyclopropyl and OH, and R^{X} is selected from —(CR^{c1}R^{d1})ₜN═S(O)R^{a1}R^{b1}, halogen and OH.

11. The compound of claim 10 or a pharmaceutically acceptable salt thereof, wherein R^{X} is selected from 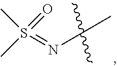 [SMILES: *N=S(C)(C)=O]
F and OH.

12. The compound of claim 1 or a pharmaceutically acceptable salt thereof, wherein Q¹ is selected from pyridinyl, pyrimidyl, pyrazinyl and phenyl.

13. The compound of claim 12 or a pharmaceutically acceptable salt thereof, wherein Q¹ is pyridinyl.

14. The compound of claim 1 or a pharmaceutically acceptable salt thereof, wherein X is CR⁴.

15. The compound of claim 14 or a pharmaceutically acceptable salt thereof, wherein R⁴ is CN.

16. The compound of claim 1 or a pharmaceutically acceptable salt thereof, wherein X is N.

17. The compound of claim 1 or a pharmaceutically acceptable salt thereof, wherein Q² is 4-7 membered heterocyclyl.

18. The compound of claim 17 or a pharmaceutically acceptable salt thereof, wherein Q² is selected from

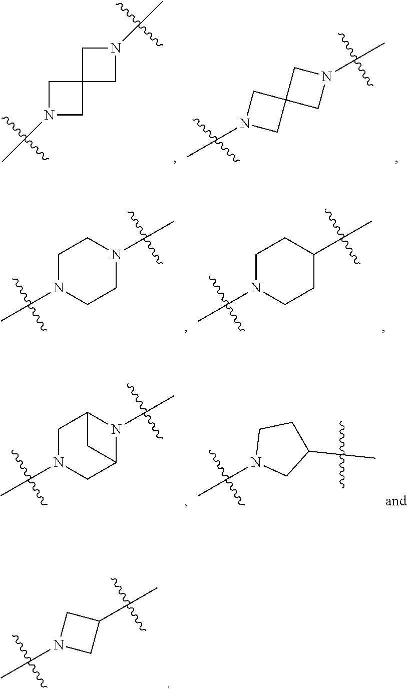

19. The compound of claim 1 or a pharmaceutically acceptable salt thereof, wherein R¹ is selected from hydrogen and halogen.

20. The compound of claim 1 or a pharmaceutically acceptable salt thereof, wherein R² is selected from hydrogen, C₁₋₁₀ alkyl, aryl-C₁₋₄ alkyl, heteroaryl-C₁₋₄ alkyl, —OR^{A2}, —C(O)R^{A2}, —C(O)OR^{A2} and —C(O)NR^{A2}R^{B2}, wherein alkyl, aryl and heteroaryl are each unsubstituted or substituted with at least one substituent, independently selected from Rx.

21. The compound of claim 20 or a pharmaceutically acceptable salt thereof, wherein R² is selected from hydrogen, ethyl, benzyl, pyridinylmethyl, Boc, —OR^{A2}, —C(O)R^{A2}, —C(O)NR^{A2}R^{B2},

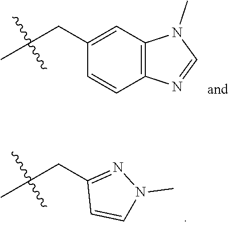

22. The compound of claim 21 or a pharmaceutically acceptable salt thereof, wherein the substituent R^{X} of ethyl, benzyl, pyridinylmethyl, 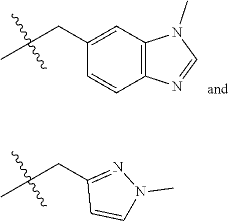
are independently selected from halogen, C₁₋₁₀ alkyl, —(CR^{c1}R^{d1})ₜNR^{a1}R^{b1}, —(CR^{c1}R^{d1})ₜS(O)ᵣR^{b} and —(CR^{c1}R^{d1})ₜOR^{b1}.

23. The compound of claim 22 or a pharmaceutically acceptable salt thereof, wherein Rx is independently selected from halogen, methyl, methoxy, dimethylamino,

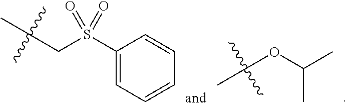

24. The compound of claim 20 or a pharmaceutically acceptable salt thereof, wherein R^{A2} is selected from hydrogen, C₁₋₁₀ alkyl, aryl, aryl-C₁₋₄ alkyl, heteroaryl and heteroaryl-C₁₋₄ alkyl, wherein the alkyl, aryl and heteroaryl in R^{A2} are each unsubstituted or substituted with at least one substituent independently selected from R^{X}.

25. The compound of claim 24 or a pharmaceutically acceptable salt thereof, wherein R^{A2} is selected from hydrogen, methyl, butyl, pentyl, pyridinyl, phenyl, methylpyridine and pyridazinyl, and the substituent R^{X} of R^{A2} is independently selected from halogen, C₁₋₁₀ alkyl, cyclopropyl, ethynyl, vinyl, —OH, methoxy, ethoxy, dimethylamino, aminomethyl, phenyl, benzyl, and 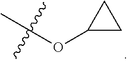 [SMILES: *OC1CC1]
wherein alkyl, phenyl and benzyl are each unsubstituted or substituted with at least one substituent independently selected from R^{Y}.

26. The compound of claim 20 or a pharmaceutically acceptable salt thereof, wherein R² is selected from hydrogen, methyl, ethyl, —OH, Boc,

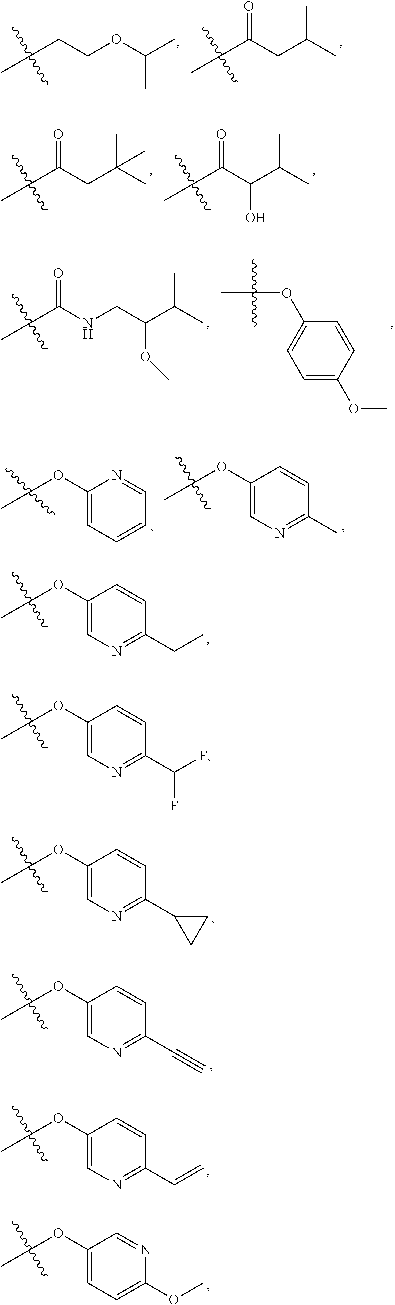

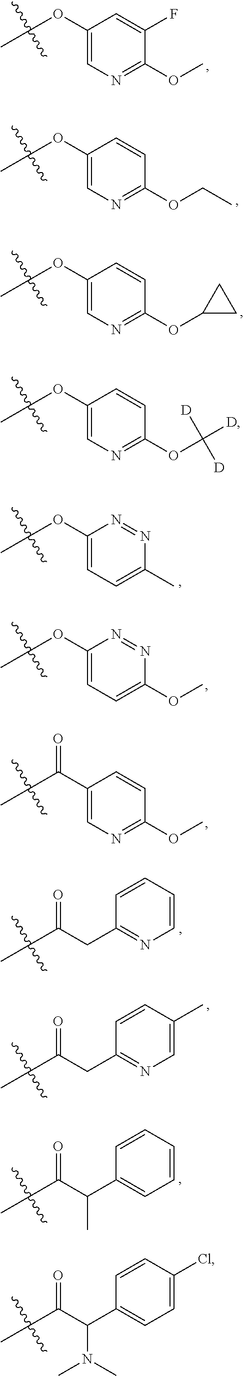

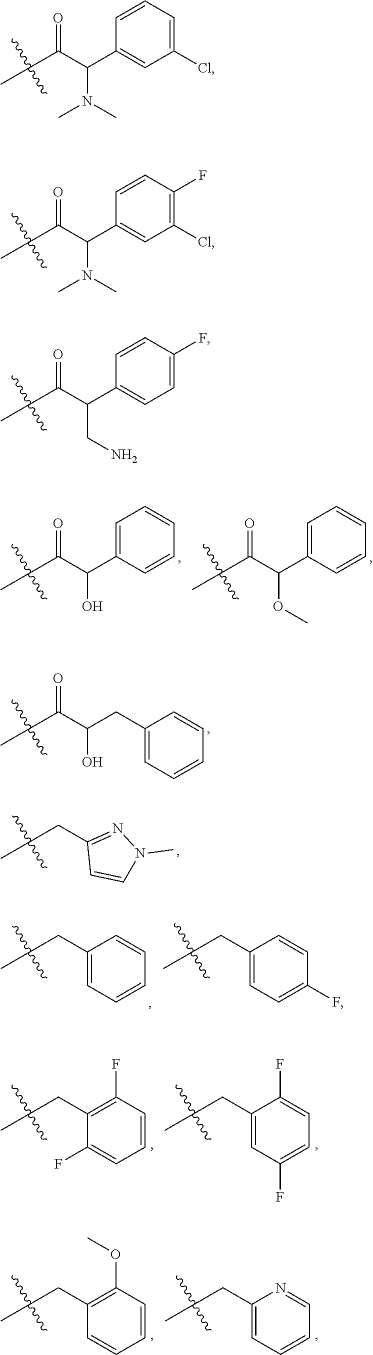

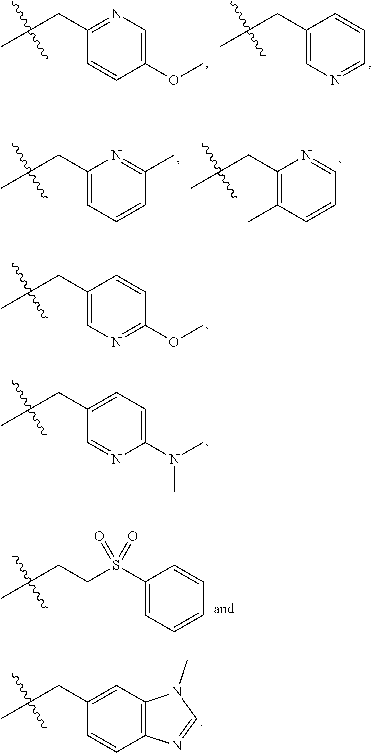

27. The compound of claim 1 selected from the group consisting of 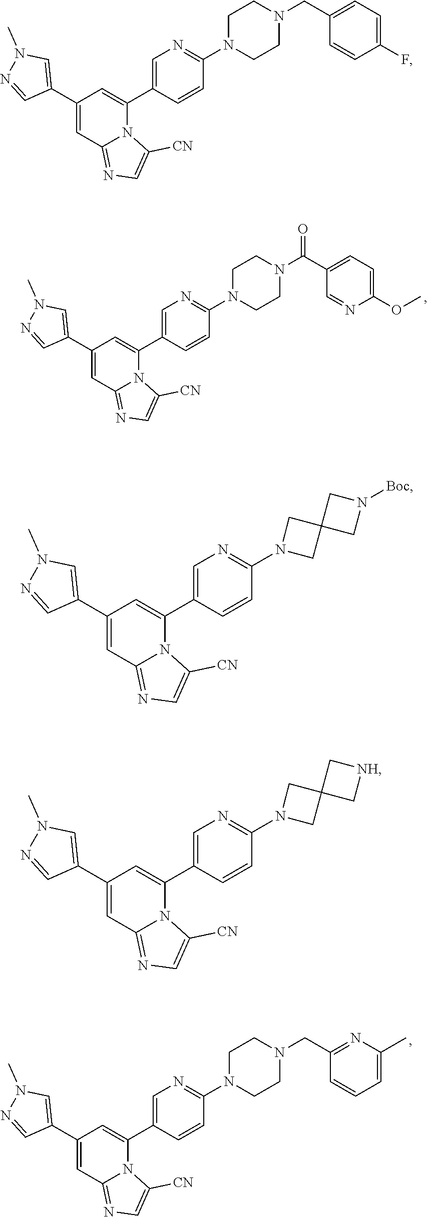 [SMILES: Cn1cc(-c2cc(-c3ccc(N4CCN(Cc5ccc(F)cc5)CC4)nc3)n3c(C#N)cnc3c2)cn1 ; COc1ccc(C(=O)N2CCN(c3ccc(-c4cc(-c5cnn(C)c5)cc5ncc(C#N)n45)cn3)CC2)cn1 ; Cn1cc(-c2cc(-c3ccc(N4CC5(CN(C(=O)OC(C)(C)C)C5)C4)nc3)n3c(C#N)cnc3c2)cn1 ; Cn1cc(-c2cc(-c3ccc(N4CC5(CNC5)C4)nc3)n3c(C#N)cnc3c2)cn1 ; Cc1cccc(CN2CCN(c3ccc(-c4cc(-c5cnn(C)c5)cc5ncc(C#N)n45)cn3)CC2)n1] 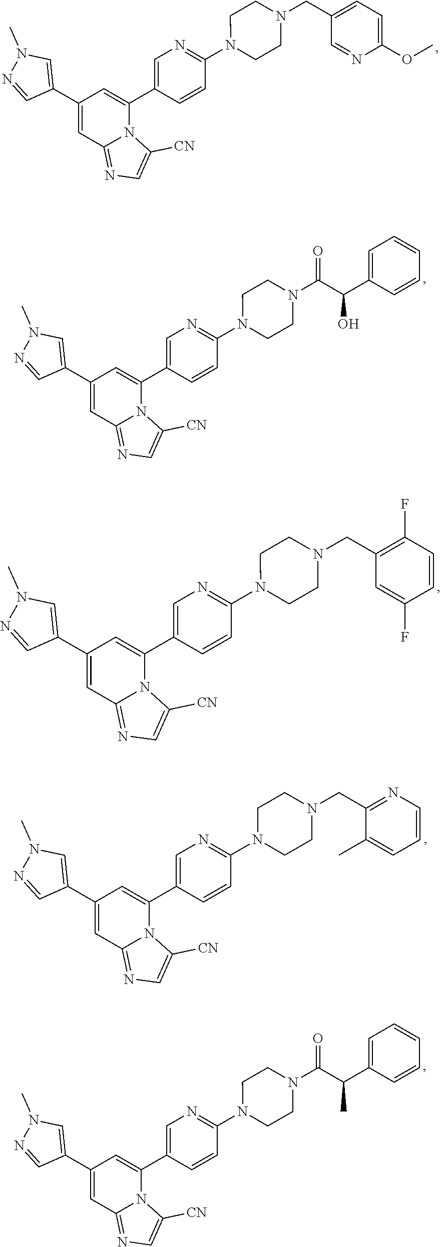 [SMILES: COc1ccc(CN2CCN(c3ccc(-c4cc(-c5cnn(C)c5)cc5ncc(C#N)n45)cn3)CC2)cn1 ; Cn1cc(-c2cc(-c3ccc(N4CCN(C(=O)[C@H](O)c5ccccc5)CC4)nc3)n3c(C#N)cnc3c2)cn1 ; Cn1cc(-c2cc(-c3ccc(N4CCN(Cc5cc(F)ccc5F)CC4)nc3)n3c(C#N)cnc3c2)cn1 ; Cc1cccnc1CN1CCN(c2ccc(-c3cc(-c4cnn(C)c4)cc4ncc(C#N)n34)cn2)CC1 ; C[C@@H](C(=O)N1CCN(c2ccc(-c3cc(-c4cnn(C)c4)cc4ncc(C#N)n34)cn2)CC1)c1ccccc1] 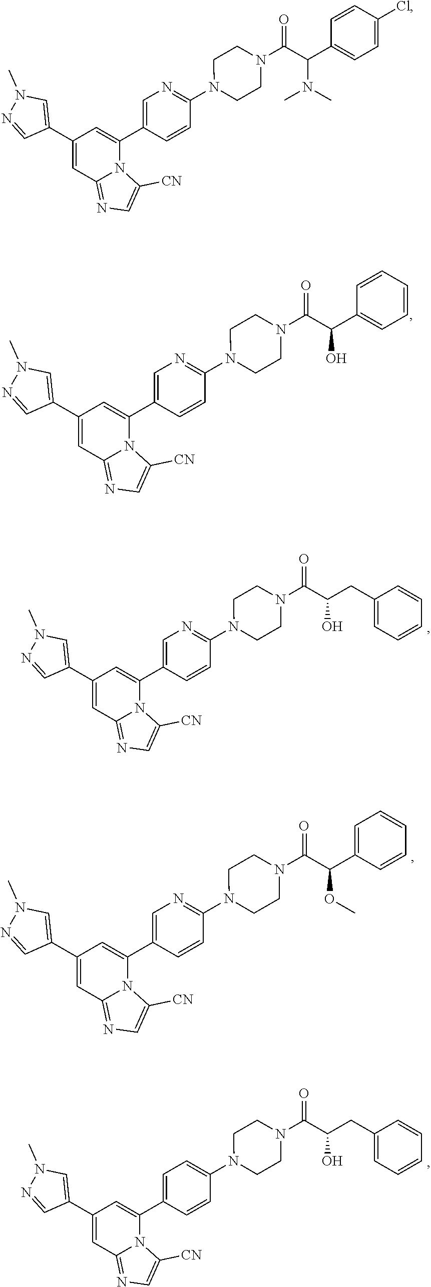 [SMILES: CN(C)C(C(=O)N1CCN(c2ccc(-c3cc(-c4cnn(C)c4)cc4ncc(C#N)n34)cn2)CC1)c1ccc(Cl)cc1 ; Cn1cc(-c2cc(-c3ccc(N4CCN(C(=O)[C@H](O)c5ccccc5)CC4)nc3)n3c(C#N)cnc3c2)cn1 ; Cn1cc(-c2cc(-c3ccc(N4CCN(C(=O)[C@@H](O)Cc5ccccc5)CC4)nc3)n3c(C#N)cnc3c2)cn1 ; CO[C@@H](C(=O)N1CCN(c2ccc(-c3cc(-c4cnn(C)c4)cc4ncc(C#N)n34)cn2)CC1)c1ccccc1 ; Cn1cc(-c2cc(-c3ccc(N4CCN(C(=O)[C@@H](O)Cc5ccccc5)CC4)cc3)n3c(C#N)cnc3c2)cn1] 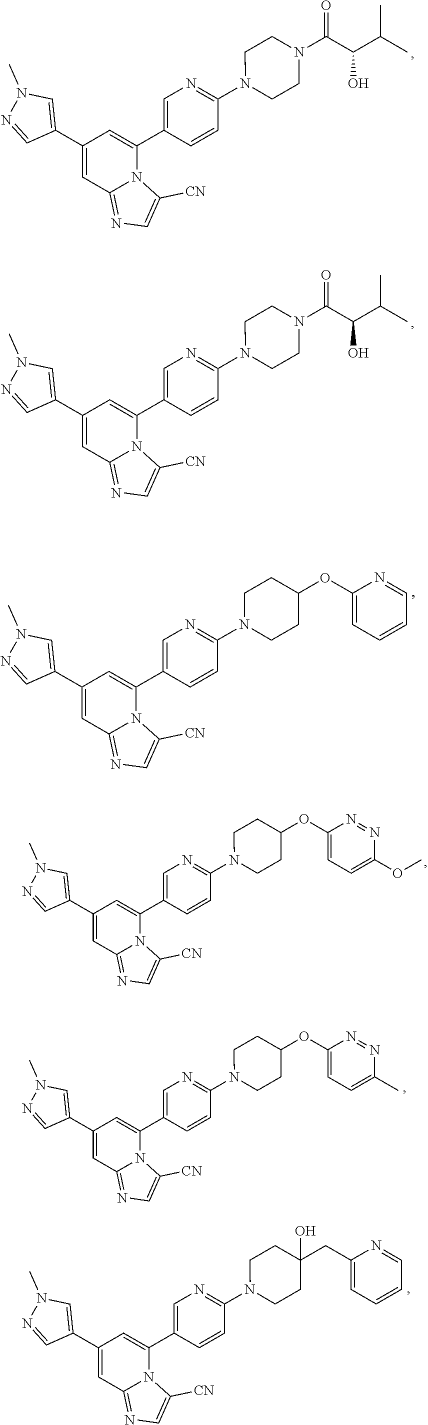 [SMILES: CC(C)[C@H](O)C(=O)N1CCN(c2ccc(-c3cc(-c4cnn(C)c4)cc4ncc(C#N)n34)cn2)CC1 ; CC(C)[C@@H](O)C(=O)N1CCN(c2ccc(-c3cc(-c4cnn(C)c4)cc4ncc(C#N)n34)cn2)CC1 ; Cn1cc(-c2cc(-c3ccc(N4CCC(Oc5ccccn5)CC4)nc3)n3c(C#N)cnc3c2)cn1 ; COc1ccc(OC2CCN(c3ccc(-c4cc(-c5cnn(C)c5)cc5ncc(C#N)n45)cn3)CC2)nn1 ; Cc1ccc(OC2CCN(c3ccc(-c4cc(-c5cnn(C)c5)cc5ncc(C#N)n45)cn3)CC2)nn1 ; Cn1cc(-c2cc(-c3ccc(N4CCC(O)(Cc5ccccn5)CC4)nc3)n3c(C#N)cnc3c2)cn1] 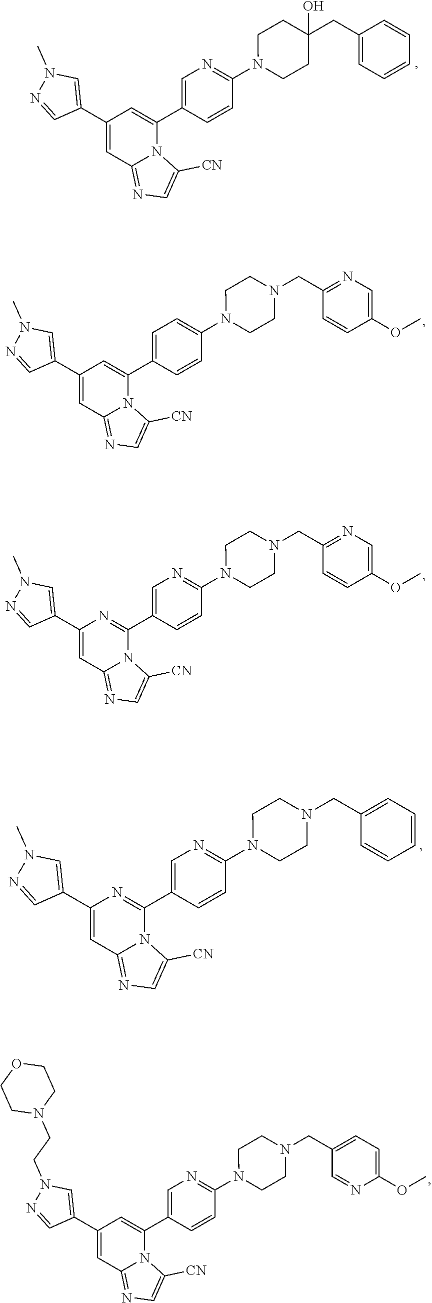 [SMILES: Cn1cc(-c2cc(-c3ccc(N4CCC(O)(Cc5ccccc5)CC4)nc3)n3c(C#N)cnc3c2)cn1 ; COc1ccc(CN2CCN(c3ccc(-c4cc(-c5cnn(C)c5)cc5ncc(C#N)n45)cc3)CC2)nc1 ; COc1ccc(CN2CCN(c3ccc(-c4nc(-c5cnn(C)c5)cc5ncc(C#N)n45)cn3)CC2)nc1 ; Cn1cc(-c2cc3ncc(C#N)n3c(-c3ccc(N4CCN(Cc5ccccc5)CC4)nc3)n2)cn1 ; COc1ccc(CN2CCN(c3ccc(-c4cc(-c5cnn(CCN6CCOCC6)c5)cc5ncc(C#N)n45)cn3)CC2)cn1] 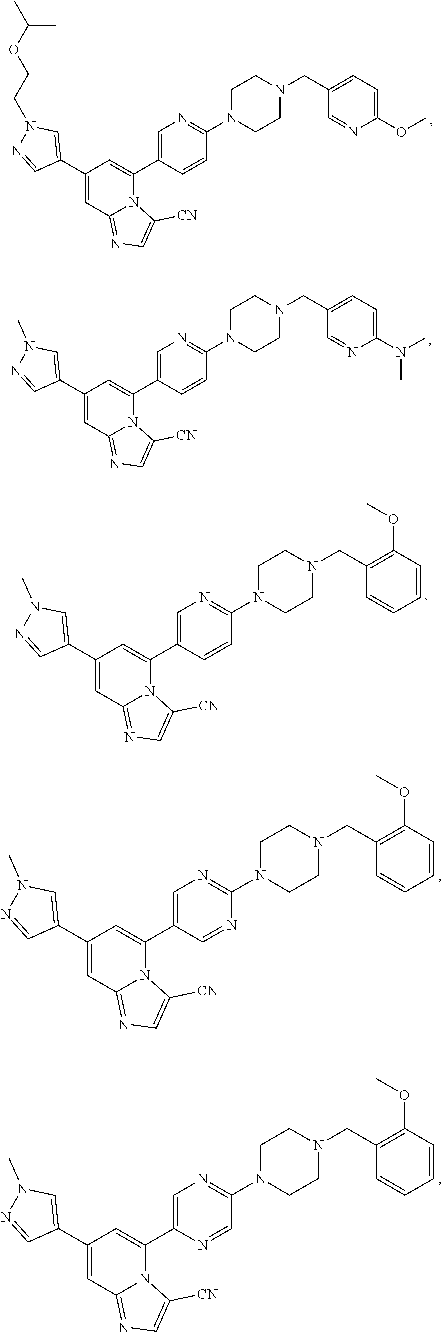 [SMILES: COc1ccc(CN2CCN(c3ccc(-c4cc(-c5cnn(CCOC(C)C)c5)cc5ncc(C#N)n45)cn3)CC2)cn1 ; CN(C)c1ccc(CN2CCN(c3ccc(-c4cc(-c5cnn(C)c5)cc5ncc(C#N)n45)cn3)CC2)cn1 ; COc1ccccc1CN1CCN(c2ccc(-c3cc(-c4cnn(C)c4)cc4ncc(C#N)n34)cn2)CC1 ; COc1ccccc1CN1CCN(c2ncc(-c3cc(-c4cnn(C)c4)cc4ncc(C#N)n34)cn2)CC1 ; COc1ccccc1CN1CCN(c2cnc(-c3cc(-c4cnn(C)c4)cc4ncc(C#N)n34)cn2)CC1] 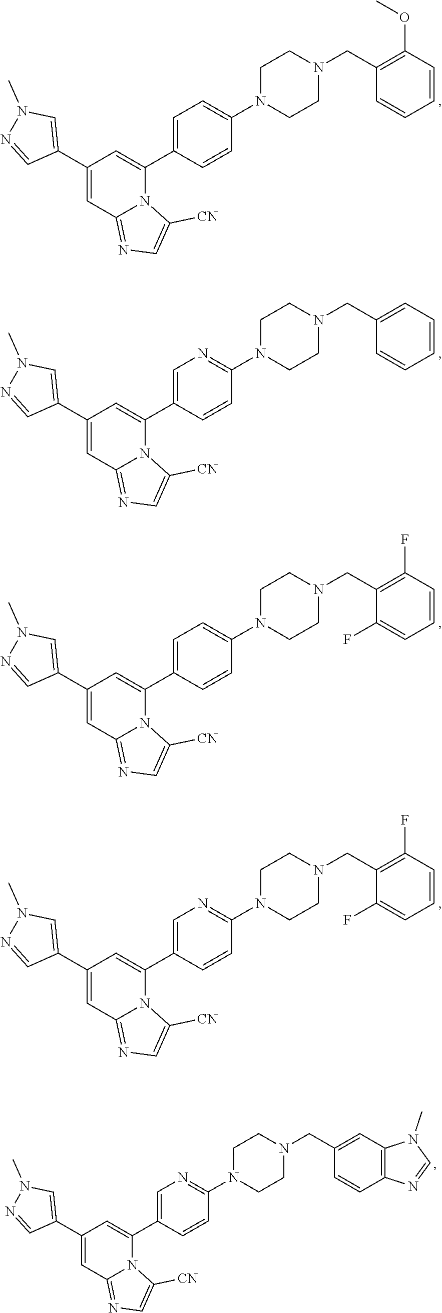 [SMILES: COc1ccccc1CN1CCN(c2ccc(-c3cc(-c4cnn(C)c4)cc4ncc(C#N)n34)cc2)CC1 ; Cn1cc(-c2cc(-c3ccc(N4CCN(Cc5ccccc5)CC4)nc3)n3c(C#N)cnc3c2)cn1 ; Cn1cc(-c2cc(-c3ccc(N4CCN(Cc5c(F)cccc5F)CC4)cc3)n3c(C#N)cnc3c2)cn1 ; Cn1cc(-c2cc(-c3ccc(N4CCN(Cc5c(F)cccc5F)CC4)nc3)n3c(C#N)cnc3c2)cn1 ; Cn1cc(-c2cc(-c3ccc(N4CCN(Cc5ccc6ncn(C)c6c5)CC4)nc3)n3c(C#N)cnc3c2)cn1] 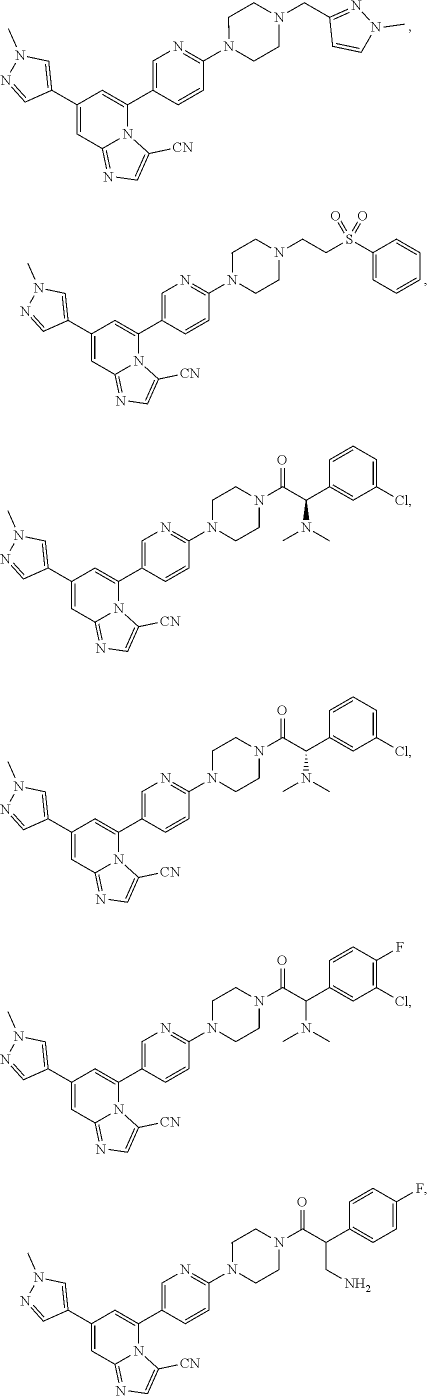 [SMILES: Cn1cc(-c2cc(-c3ccc(N4CCN(Cc5ccn(C)n5)CC4)nc3)n3c(C#N)cnc3c2)cn1 ; Cn1cc(-c2cc(-c3ccc(N4CCN(CCS(=O)(=O)c5ccccc5)CC4)nc3)n3c(C#N)cnc3c2)cn1 ; CN(C)[C@@H](C(=O)N1CCN(c2ccc(-c3cc(-c4cnn(C)c4)cc4ncc(C#N)n34)cn2)CC1)c1cccc(Cl)c1 ; CN(C)[C@H](C(=O)N1CCN(c2ccc(-c3cc(-c4cnn(C)c4)cc4ncc(C#N)n34)cn2)CC1)c1cccc(Cl)c1 ; CN(C)C(C(=O)N1CCN(c2ccc(-c3cc(-c4cnn(C)c4)cc4ncc(C#N)n34)cn2)CC1)c1ccc(F)c(Cl)c1 ; Cn1cc(-c2cc(-c3ccc(N4CCN(C(=O)C(CN)c5ccc(F)cc5)CC4)nc3)n3c(C#N)cnc3c2)cn1] 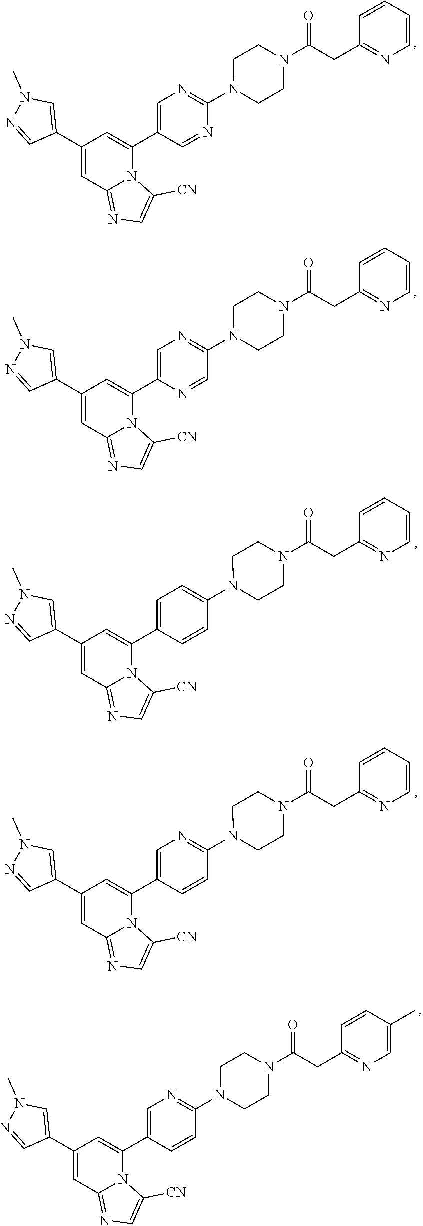 [SMILES: Cn1cc(-c2cc(-c3cnc(N4CCN(C(=O)Cc5ccccn5)CC4)nc3)n3c(C#N)cnc3c2)cn1 ; Cn1cc(-c2cc(-c3cnc(N4CCN(C(=O)Cc5ccccn5)CC4)cn3)n3c(C#N)cnc3c2)cn1 ; Cn1cc(-c2cc(-c3ccc(N4CCN(C(=O)Cc5ccccn5)CC4)cc3)n3c(C#N)cnc3c2)cn1 ; Cn1cc(-c2cc(-c3ccc(N4CCN(C(=O)Cc5ccccn5)CC4)nc3)n3c(C#N)cnc3c2)cn1 ; Cc1ccc(CC(=O)N2CCN(c3ccc(-c4cc(-c5cnn(C)c5)cc5ncc(C#N)n45)cn3)CC2)nc1] 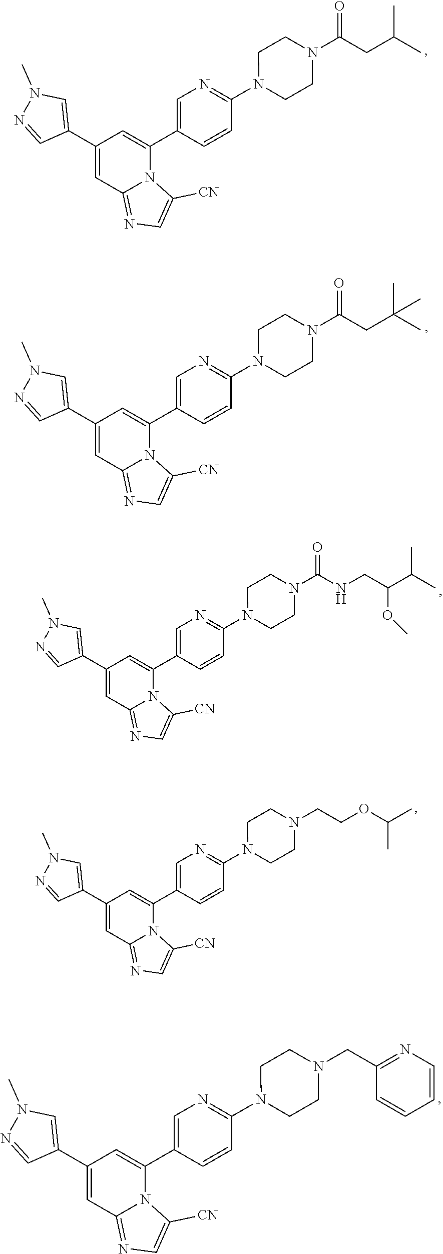 [SMILES: CC(C)CC(=O)N1CCN(c2ccc(-c3cc(-c4cnn(C)c4)cc4ncc(C#N)n34)cn2)CC1 ; Cn1cc(-c2cc(-c3ccc(N4CCN(C(=O)CC(C)(C)C)CC4)nc3)n3c(C#N)cnc3c2)cn1 ; COC(CNC(=O)N1CCN(c2ccc(-c3cc(-c4cnn(C)c4)cc4ncc(C#N)n34)cn2)CC1)C(C)C ; CC(C)OCCN1CCN(c2ccc(-c3cc(-c4cnn(C)c4)cc4ncc(C#N)n34)cn2)CC1 ; Cn1cc(-c2cc(-c3ccc(N4CCN(Cc5ccccn5)CC4)nc3)n3c(C#N)cnc3c2)cn1] 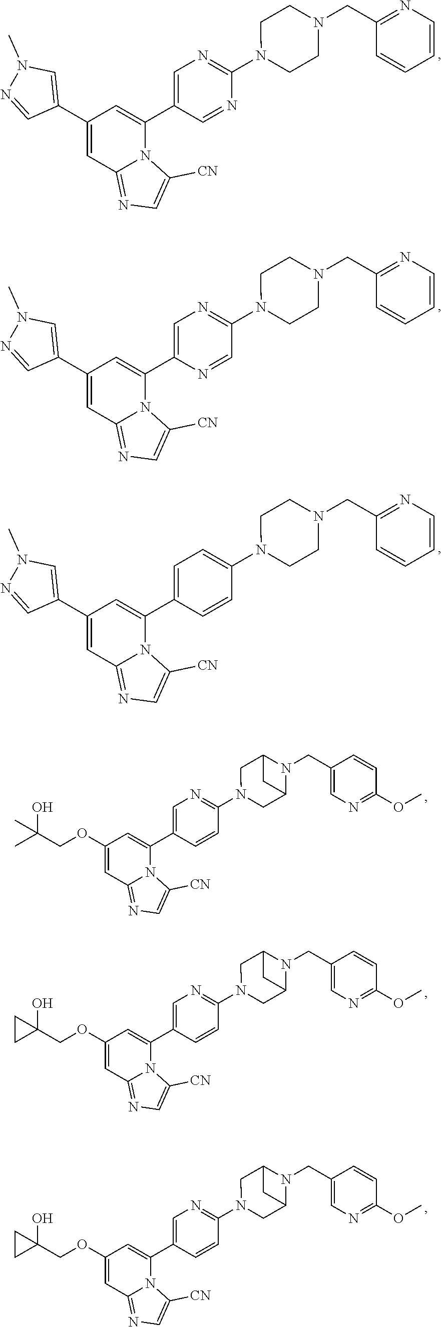 [SMILES: Cn1cc(-c2cc(-c3cnc(N4CCN(Cc5ccccn5)CC4)nc3)n3c(C#N)cnc3c2)cn1 ; Cn1cc(-c2cc(-c3cnc(N4CCN(Cc5ccccn5)CC4)cn3)n3c(C#N)cnc3c2)cn1 ; Cn1cc(-c2cc(-c3ccc(N4CCN(Cc5ccccn5)CC4)cc3)n3c(C#N)cnc3c2)cn1 ; COc1ccc(CN2C3CC2CN(c2ccc(-c4cc(OCC(C)(C)O)cc5ncc(C#N)n45)cn2)C3)cn1 ; COc1ccc(CN2C3CC2CN(c2ccc(-c4cc(OCC5(O)CC5)cc5ncc(C#N)n45)cn2)C3)cn1 ; COc1ccc(CN2C3CC2CN(c2ccc(-c4cc(OCC5(O)CC5)cc5ncc(C#N)n45)cn2)C3)cn1] 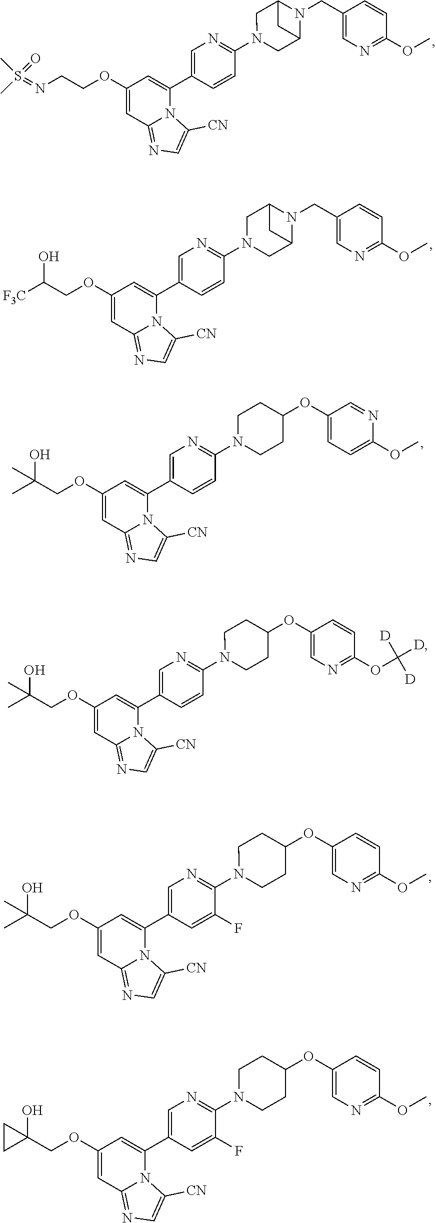 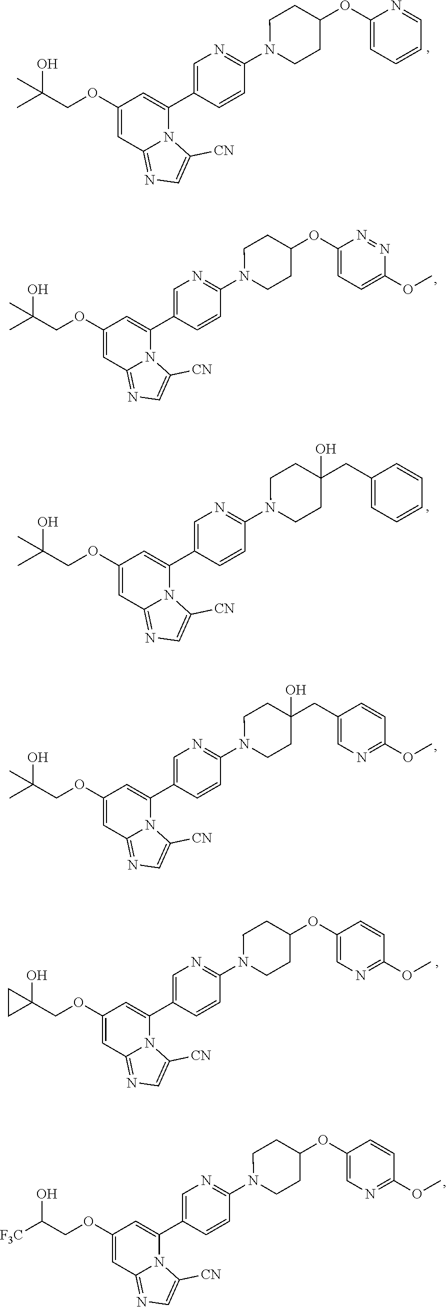 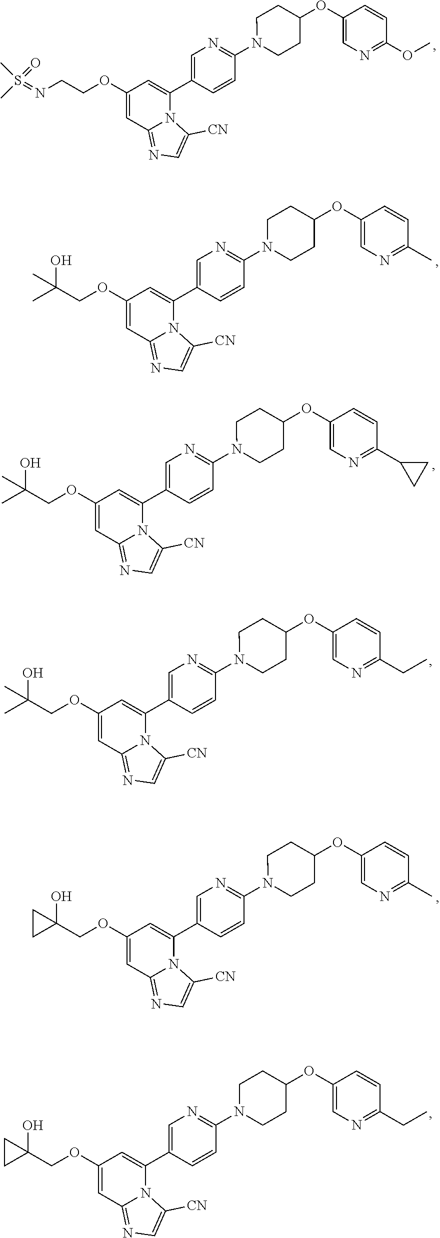 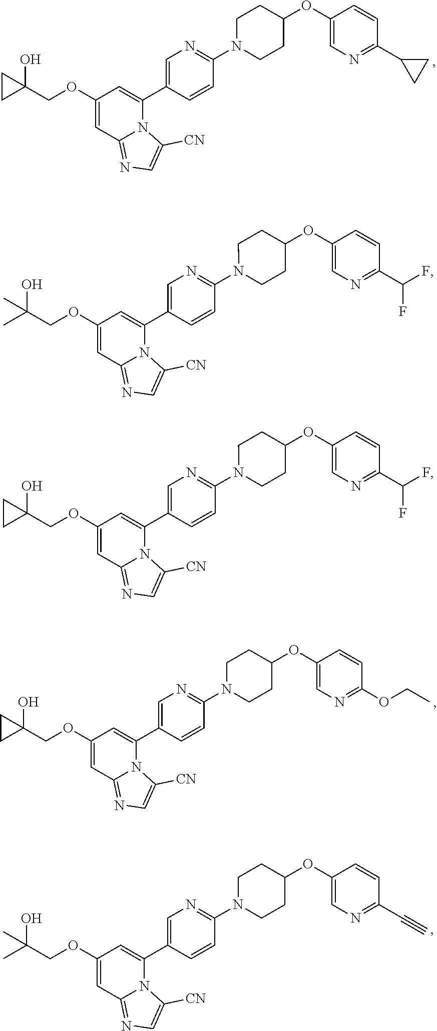 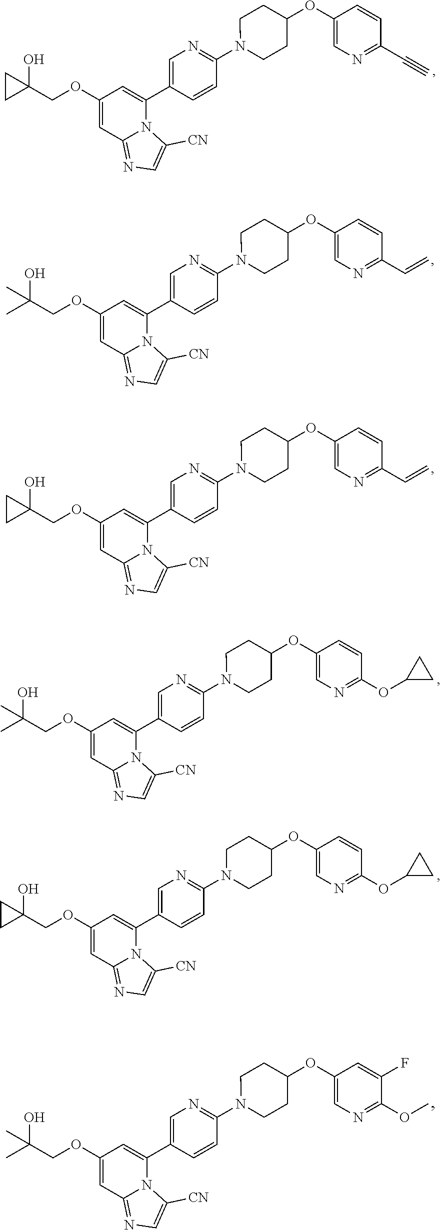 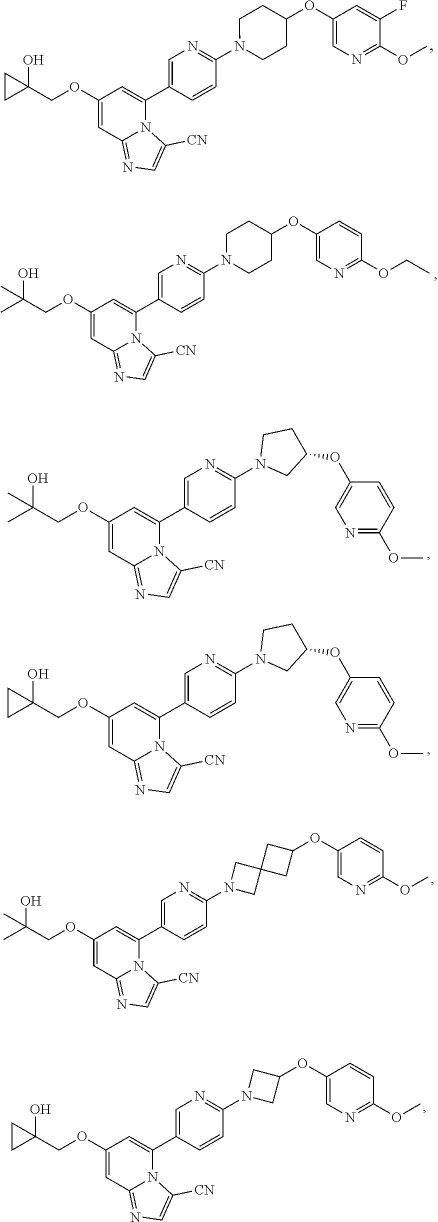 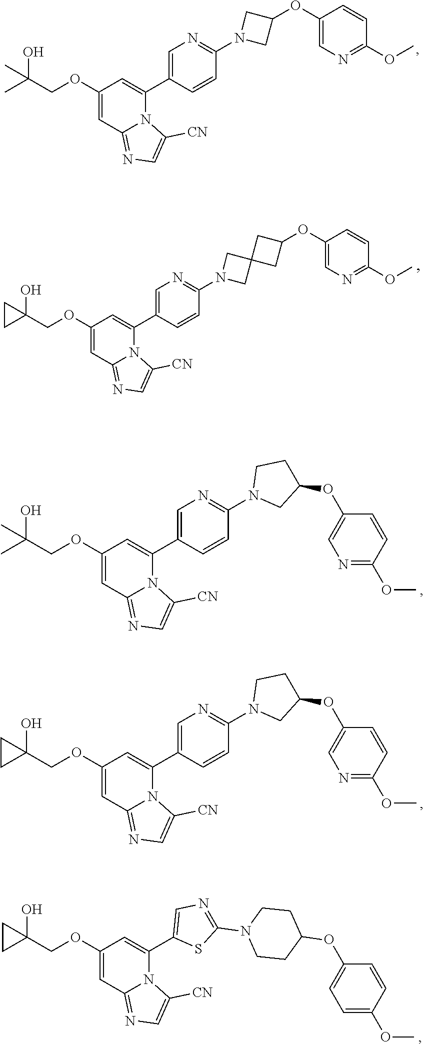
or pharmaceutically acceptable salts thereof.

28. A pharmaceutical composition, comprising the compound of claim 1 or a pharmaceutically acceptable salt thereof and at least one pharmaceutically acceptable carrier.

29. A method of treating, ameliorating or preventing a condition, which responds to inhibition of RET, comprising administering to a subject in need of such treatment an effective amount of the compound of claim 1, or a pharmaceutically acceptable salt thereof, or of at least one pharmaceutical composition thereof, and optionally in combination with a second therapeutic agent.

30. (canceled)
